(19) United States
(12) Patent Application Publication
(10) Pub. No.: US 2010/0311748 A1
(43) Pub. Date: Dec. 9, 2010

(54) HETEROCYCLIC AMIDES USEFUL FOR THE TREATMENT OF CANCER AND PSORIASIS

(75) Inventors: Leslie Dakin, Waltham, MA (US); Benjamin Fauber, Waltham, MA (US); Alexander Hird, Waltham, MA (US); James Janetka, Waltham, MA (US); Daniel John Russell, Waltham, MA (US); Qibin Su, Waltham, MA (US); Bin Yang, Waltham, MA (US); Xiaolan Zheng, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/675,728

(22) PCT Filed: Aug. 29, 2008
(86) PCT No.: PCT/GB2008/050756

§ 371 Date: Mar. 1, 2010

Related U.S. Application Data
(60) Provisional application No. 60/969,364, filed on Aug. 31, 2007.
(60) Provisional application No. 61/036,658, filed on Mar. 14, 2008.

(51) Int. Cl. A61K 31/5377; C07D 401/12; A61K 31/4439; C07D 417/14; A61K 31/496; C07D 513/04; A61K 31/444; A61K 31/497; A61K 31/501; C07D 233/64; A61K 31/4164; A61P 35/00

(52) U.S. Cl. 514/235.8; 546/272.7; 514/341; 544/364; 514/253.1; 546/273.4; 546/270.1; 514/338; 546/261; 514/335; 544/405; 514/255.05; 544/131; 544/238; 514/236.8; 514/252.03; 548/343.5; 514/400

(57) ABSTRACT

The present disclosure relates to heterocyclic amide compounds, which are useful for inhibiting the Hedgehog pathway, and their use in treating a disease or medical condition mediated alone or in part by Hedgehog pathway inhibition. Also disclosed are methods for manufacture of these compounds, pharmaceutical compositions including these compounds, and use of these compounds in the manufacture of medicaments for treating such diseases and medical conditions in a subject. Formula (IA) with the provisio that either R₂ or R₃ is (Z).

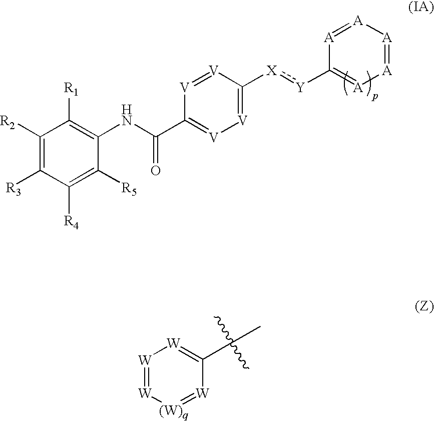

### RELATED APPLICATIONS

This application claims priority under 35 U.S.C. §119(e) to U.S. Provisional Application Nos. 60/969,364 filed on Aug. 31, 2007 and 61/036,658, filed on Mar. 14, 2008; the entire contents of each of which is hereby expressly incorporated by reference in their entireties.

### BACKGROUND OF THE INVENTION

The Hedgehog pathway (HH pathway) is a well-studied pathway affecting numerous biological processes, such as embryogenesis, where the pathway is activated and mediates patterning of the embryo, cell differentiation and proliferation. This pathway has been conserved throughout evolution, and components of the pathway have been identified in many species including sea urchins, worms, flies, and mammals. Much of the current understanding about the HH pathway has come from studies in Drosophila. The human genome contains three hedgehog genes: Sonic (SHH), Indian (IHH) and Desert (DHH). Sonic Hedgehog is the most widely expressed of the three genes, and studies have shown that this gene plays a role in many aspects of embryogenesis.

The Sonic gene codes for the SHH protein ligand. All hedgehog proteins are secreted from the cell and bind to their common 12-pass transmembrane protein, PTCH1, whose function is to inhibit a 7-pass GPCR-like membrane protein called Smoothened (SMO). The binding of SHH to PTCH1 relieves the inhibition on SMO, allowing translocation of SMO to the membrane followed by subsequent initiation of a signal transduction pathway (Varjosalo et al., J. Cell Sci. 120:3-6 (2007)). Recently, it has been shown that the localization of SMO to the membrane occurs specifically in the cilia of mammalian cells (Caspary et al., Dev. Cell 12:767-778 (2007)). Moreover, mutations in intraflagellar transport proteins result in dysfunctional SHH signaling and lead to developmental deformities analogous to those observed with SHH mutations. After the HH pathway is activated, a complex series of interactions downstream of SMO ultimately leads to the processing and translocation of the GLI transcription factors to the nucleus, where they act as transcriptional regulators. In vertebrates, there are three GLI genes (GLI1, GLI2, and GLI3) which are members of the zinc finger transcription factor family. GLI1 and GLI2 act primarily as transcriptional activators, while GLI3 functions as a transcriptional repressor. The GLI2 gene is constitutively expressed and is believed to be the primary target for activation by SMO. In the presence of SHH ligand and activation of SMO, the GLI2 protein becomes stabilized and functions to up-regulate a number of genes identified as targets of the HH pathway, including GLI1, PTCH, BCL2, c-myc and IGF2. Of these genes, studies have indicated that Gill appears to be the most reliable biological endpoint for measuring activation of the HH pathway.

One of the difficulties in targeting the HH pathway is the incomplete understanding of the signal transduction pathway and the lack of identification of positive pathway regulators. Cyclopamine is a well-established natural product antagonist of the HH pathway, which has been proven to be a valuable tool to modulate the HH pathway. Cyclopamine has been shown to directly bind to SMO and inhibit its activation, leading to downregulation of the pathway both in vitro and in vivo (Chen et al., Cancer Sci. 98:68-76 (2007); Mukherjee et al., Cancer Bio & Therapy 5:674-683 (2006)).

Recently, the linkage of the Hedgehog pathway to diseases, such as cancer, has been established. Activating mutations in either PTCH or SMO have been associated with basal cell carcinoma, medulloblastoma, and rhabdomyosarcoma. In addition, upregulation of the pathway, as measured by overexpression of SHH or upregulation of Gill expression, has been associated with solid tumors including prostate, pancreas, upper digestive tract tumors and small cell lung cancer (Bak et al., Pharmacogenomica 4:411-429 (2003)). In addition, several transgenic or knockout/knock-in models have been developed by overexpression of pathway components in specific tissues or tissue specific knockout that lead to tumor formation in mice. For example, overexpression of constitutively active SMO in the mammary gland leads to increased proliferation, altered differentiation and ductal dysplasia (Moraes et al., Development 134:1231-1242 (2007)). Mice heterozygous for PTCH form basal cell carcinoma when exposed to UV light (Aszterbaum et al. Nat. Med. 5:1285-1291 (1999)), and tissue specific overexpression of SHH in the pancreas leads to abnormal tubule structures that mimic human pancreatic cancer (Thayer et al., Nature 425:851-856 (2003)). In addition, several studies have reported expression of HH signaling components in human tumor tissues including, but not limited to, prostate, pancreas, ovarian, melanoma, breast, colon, lung, esophagus, stomach, biliary, hepatocellular and multiple myeloma.

The tumor microenvironment is a very important aspect of tumorogenesis, but it is unclear as to how growth factor signaling pathways influence the tumor microenvironment. These pathways may function in an autocrine manner, where the ligands are produced by the tumor cells and thus activate the signaling pathways within the tumor cell. However, during normal development, the HH pathway is thought to function in a paracrine manner where the reactive stromal cells produce the growth factors and send signals back to the developing tumor (Fan et al., Endocrinology 145:3961-3970 (2004).

In addition to effects on proliferation and differentiation, the HH pathway is also implicated in the process of angiogenesis, which results in the growth of new blood vessels from existing vasculature and remodeling smaller vessels into larger ones. All of these effects help to promote growth and survival of the tumor (Klagsbrun and D'Amore, Annu Rev. Physiol. 53:217-239 (1991); Cherington et al., Adv. Cancer Res. 79:1-38 (2000)).

In addition, the HH pathway may play a role in the developing field of cancer stem cells. Stem cells are slowly replicating cells that have the ability to give rise to exact replicates of themselves, as well as a heterogeneous population of progeny cells. In the stem cell model of cancer, a rare subpopulation of cells have the ability to self-renew, yielding another malignant stem cell as well as non-tumorigenic cancer cells, thus increasing the heterogeneous cell population of the tumor. Recent studies have demonstrated in leukemia and several solid tumors including brain, prostate, pancreatic, colon and breast, that a small proportion of cancer cells have the capacity to proliferate extensively and form new heterogenous tumors in vivo (Clarke et al. Cancer Res. 66:9339-9344 (2006). For example, in the pancreas, these cancer stem cells have also been reported to have a higher level of GLI expression (Li et al., Cancer Res. 67:1030-1037 (2007)). Compounds effectively inhibiting the Hedgehog pathway could thus be useful in decreasing cancer stem cell proliferation or differentiation activity.

### SUMMARY OF THE INVENTION

Accordingly, novel compounds are provided that are potent inhibitors and effectors of the Hedgehog pathway, and therefore possess the ability to prevent gene transcription effected by the GLI proteins. This inhibitory ability results in preventing or reducing cell differentiation, proliferation, and/or affecting stromal microenvironment modulation. The disclosed compounds are useful for treating diseases and medical conditions mediated alone or in part by Hedgehog pathway inhibition, and thus possess anti-proliferative (such as anti-cancer) activity. Such activity is useful in treating subjects having a PTCH loss-of function phenotype, a SMO gain-of-function phenotype or a Hedgehog gain-of-function phenotype.

One aspect of the invention provides a compound of formula IA

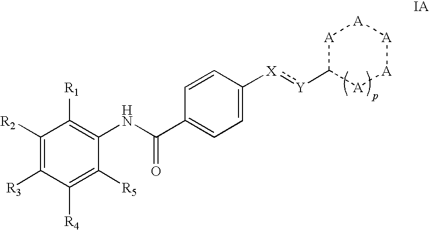

wherein
[symbol] represents a single bond or a double bond;
[symbol] represents a single bond, a double bond, a triple bond, or when X or Y is a direct bond [symbol] represents the absence of a bond;

R₁, R₂, R₃, and R₄ are each independently selected from the group consisting of hydrogen, C₁₋₆alkoxy, C₁₋₆alkoxyC₁₋₆alkyl, C₁₋₆alkyl, aminoC₁₋₆alkyl, C₃₋₈cycloalkyl, cyano, haloC₁₋₆alkyl, halogen, hydroxy, sulfonyl, sulfide, and thio,

with the proviso that either R₂ or R₃ is Z;

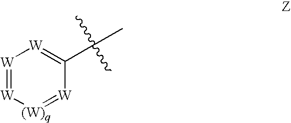

each W is independently selected from the group consisting of CR₁₀, NR₁₀, N, O, and S, where R₁₀ is selected from the group consisting of hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, C₃₋₆cycloalkyl, hydroxy, hydroxyC₁₋₆alkyl, nitro, sulfide, sulfonamido, and sulfonyl, or

two adjacent W atoms can be taken together with their R₁₀ substituents to form a fused second ring, wherein the second ring is selected from the group consisting of aryl, C₃₋₈cycloalkyl, a 5- or 6-membered heteroaryl, and a 5- or 6-membered heterocyclyl;

q is 0 or 1, where

if q is 0 and two adjacent W atoms taken together with their R₁₀ substituents form a bicycle selected from the group consisting of benzimidazolyl, benzoxazolyl, benzothiazolyl, and oxazolopyridyl, then at least one A is N,

if q is 1, two W are N, and two adjacent W atoms taken together with their R₁₀ substituents form a quinoxalinyl, then at least one A is N, and

if q is 1 and each W is CR₁₀, then two adjacent W atoms are taken together with their R₁₀ substituents to form a second ring selected from the group consisting of a 5- or 6-membered heteroaryl and a 5- or 6-membered heterocyclyl;

R₅ is selected from the group consisting of alkyl, haloC₁₋₆alkyl, and halogen;

R₆, R₇, R₈ and R₉ are each independently selected from the group consisting of hydrogen, C₁₋₆alkyl, amino, C₃₋₈cycloalkyl, C₁₋₆alkoxy, cyano, haloC₁₋₆alkyl, halogen, sulfide, sulfonyl, and sulfonamido;

when joined by a single bond, X and Y are each independently selected from the group consisting of O, S, SO₂, NR₁₁, and CR₁₁R₁₂, or one of X and Y can be a direct bond,

when joined by a double bond, X and Y are each independently CR₁₁, and

when joined by a triple bond, X and Y are each C;

each R₁₁ and R₁₂ are each independently selected from the group consisting of hydrogen, C₁₋₆alkoxy, C₁₋₆alkyl, amino, cyano, haloC₁₋₆alkyl, halogen, and sulfide;

each A is selected from the group consisting of CR₁₃, CR₁₃R₁₃, NR₁₃, N, O, and S;

each R₁₃ is selected from the group consisting of hydrogen, C₁₋₆alkoxy, C₁₋₆alkoxyamino, C₁₋₆alkoxyC₁₋₆alkyl, C₁₋₆alkoxycarbonyl, C₁₋₆alkyl, C₁₋₆alkylamino, amidino, amido, amino, aminoC₁₋₆alkylamino, aryl, aryloxy, carboxamido, C₃₋₈cycloalkyl, C₃₋₈cycloalkylC₁₋₆alkoxy, cyano, haloC₁₋₆alkyl, halogen, heterocyclyl, heterocyclylC₁₋₆alkyl, heterocyclylC₁₋₆alkoxy, hydroxy, hydroxyC₁₋₆alkyl, hydroxyC₁₋₆alkoxy, nitro, sulfide, sulfonamido, and sulfonyl;

p is 0 or 1, where

if p is 0, then two adjacent A atoms can be taken together with their R₁₃ substituents to form a fused second ring, wherein the second ring is selected from the group consisting of aryl, 6-membered heteroaryl and 6-membered heterocyclyl, and

if p is 1, then two adjacent A atoms can be taken together with their R₁₃ substituents to form a fused second ring, wherein the second ring is selected from the group consisting of aryl, 5- or 6-membered heteroaryl and 5- or 6-membered heterocyclyl;

or a pharmaceutically acceptable salt thereof.

In another aspect, the invention provides a compound of formula II

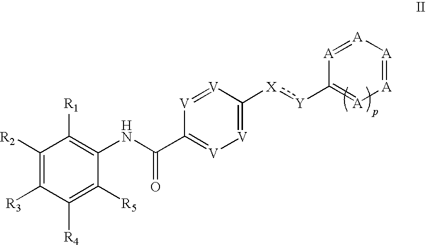

wherein

R₁, R₂, R₃, and R₄ are each independently selected from the group consisting of hydrogen, C₁₋₆alkoxy, C₁₋₆alkoxyC₁₋₆alkyl, C₁₋₆alkyl, aminoC₁₋₆alkyl, C₃₋₈cycloalkyl, cyano, haloC₁₋₆alkyl, halogen, hydroxy, sulfonyl, sulfide, and thio,

with the proviso that either R₂ or R₃ is Z;

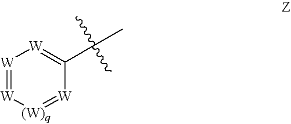

each W is independently selected from the group consisting of CR₁₀, NR₁₀, N, O, and S, where R₁₀ is selected from the group consisting of hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, C₃₋₆cycloalkyl, hydroxy, hydroxyC₁₋₆alkyl, nitro, sulfide, sulfonamido, and sulfonyl, or

two adjacent W atoms can be taken together with their R₁₀ substituents to form a fused second ring, wherein the second ring is selected from the group consisting of aryl, C₃₋₈cycloalkyl, a 5- or 6-membered heteroaryl, and a 5- or 6-membered heterocyclyl;

q is 0 or 1, where

if q is 0 and two adjacent W atoms taken together with their R₁₀ substituents form a bicycle selected from the group consisting of benzimidazolyl, benzoxazolyl, benzothiazolyl, and oxazolopyridyl, then at least one A is N,

if q is 1, two W are N, and two adjacent W atoms taken together with their R₁₀ substituents form a quinoxalinyl, then at least one A is N, and

if q is 1 and each W is CR₁₀, then two adjacent W atoms are taken together with their R₁₀ substituents to form a second ring selected from the group consisting of a 5- or 6-membered heteroaryl and a 5- or 6-membered heterocyclyl;

R₅ is selected from the group consisting of alkyl, haloC₁₋₆alkyl, and halogen;

when joined by a single bond, X and Y are each independently selected from the group consisting of O, S, SO₂, NR₁₁, and CR₁₁R₁₂, or one of X and Y can be a direct bond,

when joined by a double bond, X and Y are each independently CR₁₁, and

when joined by a triple bond, X and Y are each C;

each R₁₁ and R₁₂ are each independently selected from the group consisting of hydrogen, C₁₋₆alkoxy, C₁₋₆alkyl, amino, cyano, haloC₁₋₆alkyl, halogen, and sulfide;

each A is selected from the group consisting of CR₁₃, NR₁₃, N, O, and S;

each R₁₃ is selected from the group consisting of hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, C₃₋₈cycloalkyl, cyano, haloC₁₋₆alkyl, halogen, heterocyclylC₁₋₆alkyl, hydroxy, hydroxyC₁₋₆alkyl, nitro, sulfide, sulfonamido, and sulfonyl;

each V is independently selected from the group consisting of CR₁₄ and N;

each R₁₄ is selected from the group consisting of hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, hydroxy, hydroxyC₁₋₆alkyl, nitro, sulfide, sulfonamido, and sulfonyl;

p is 0 or 1, where

if p is 0, then two adjacent A atoms can be taken together with their R₁₃ substituents to form a fused second ring, wherein the second ring is selected from the group consisting of aryl, 6-membered heteroaryl and 6-membered heterocyclyl; and

if p is 1, then two adjacent A atoms can be taken together with their R₁₃ substituents to form a fused second ring, wherein the second ring is selected from the group consisting of aryl, 5- or 6-membered heteroaryl and 5- or 6-membered heterocyclyl,

or a pharmaceutically acceptable salt thereof.

An additional aspect of the invention provides a compound of formula III

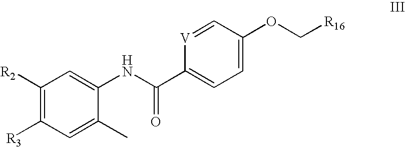

wherein

V is N or CH;

R₂ is selected from the group consisting of pyrazolyl, imidazolyl, benzoimidazol, thiazolyl, pyridyl, triazolyl, purinyl, and quinoxalinyl, wherein R₂ is optionally substituted with one or more R₁₅;

R₁₅ may be selected from the group consisting of alkyl, nitro, aryl, heteroaryl wherein R₁₅ may be optionally substituted with halo, alkyl, alkoxy, alkylthio, aryl, and heteroaryl;

R₃ is selected from the group consisting of hydrogen and alkyl;

R₁₆ is selected from the group consisting of aryl and heterocyclyl wherein R₁₆ is optionally substituted with R₁₇; and

R₁₇ is selected from the group consisting of halo, alkyl, alkoxy, alkylthio, wherein R₁₇ is optionally substituted with aryl or heteroaryl,

or a pharmaceutically acceptable salt thereof.

In yet another aspect, the invention provides a compound of formula IV

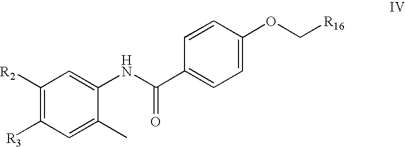

wherein

R₂ is selected from the group consisting of thiazol-2-yl, quinoxalin-2-yl, phenyl, benzothiazol-2-yl, 7H-purin-6-yl, 6-aminopyridazin-3-yl, 6-amino-2-pyridyl, 5-nitro-1H-benzoimidazol-2-yl, 5-methyl-3H-imidazol-4-yl, 5-methyl-1H-imidazol-4-yl, 5-methyl-1,3,4-oxadiazol-2-yl, 5-methyl-1,2,4-oxadiazol-3-yl, 5-ethoxycarbonyl-4-methyl-thiazol-2-yl, 5-aminopyrazin-2-yl, 5-amino-2-pyridyl, 5-[(4-methylpiperazin-1-yl)methyl]thiazol-2-yl, 5,7-diazabicyclo[4.3.0]nona-2,4,8,10-tetraen-4-yl, 5-(trifluoromethyl)-2H-pyrazol-3-yl, 5-(pyrazol-1-ylmethyl)thiazol-2-yl, 5-(morpholinomethyl)thiazol-2-yl, 5-(hydroxymethyl)-1-methyl-imidazol-4-yl, 4-thiazol-2-yl-1H-imidazol-2-yl, 4-thia-1,6-diazabicyclo[3.3.0]octa-2,5,7-trien-7-yl, 4-tert-butyl-1H-imidazol-2-yl, 4-pyridyl, 4-phenyl-1H-imidazol-2-yl, 4-methyl-3H-imidazol-2-yl, 4-methyl-1H-imidazol-2-yl, 4-ethyl-1H-imidazol-2-yl, 4-cyclopropyl-1H-imidazol-2-yl, 4,5-dimethyl-1,2,4-triazol-3-yl, 4-(trifluoromethyl)-3H-imidazol-2-yl, 4-(hydroxymethyl)-1H-imidazol-2-yl, 4-(4-pyrrolidin-1-ylphenyl)-1H-imidazol-2-yl, 4-(3-pyridyl)-1H-imidazol-2-yl, 3-pyridyl, 3-methylimidazol-4-yl, 2-pyridyl, 2-methylpyrazol-3-yl, 2-methyl-1H-imidazol-4-yl, 2,4-dimethylthiazol-5-yl, 2,3-dimethylimidazol-4-yl, 1-methylpyrazol-4-yl, 1-methylimidazol-4-yl, 1-methylimidazol-2-yl, 1-methyl-5-(methylaminomethyl)imidazol-4-yl, 1-isobutylpyrazol-4-yl, 1H-triazol-4-yl, 1H-imidazol-4-yl, 1H-imidazol-2-yl, 1H-benzoimidazol-2-yl, 1-[(3-bromo-2-pyridyl)methyl]imidazol-2-yl, 1,5-dimethylimidazol-2-yl, 1,4-dimethylimidazol-2-yl, 1,3,5-trimethylpyrazol-4-yl, 1,2-dimethylimidazol-4-yl;

R₃ is selected from the group consisting of hydrogen, methyl, and 1H-benzoimidazol-2-yl; and

R₁₆ is selected from the group consisting of 2-cyanophenyl, 2-methoxyphenyl, 3,4-dimethoxy-2-pyridyl, 3,5-dimethoxyphenyl, 3-cyanophenyl, 3-methoxyphenyl, 4-fluorophenyl, 4-methylsulfonylphenyl, 6-chlorobenzo[1,3]dioxol-5-yl, 2-(trifluoromethyl)phenyl, 3-(2-morpholinoethoxy)phenyl, 4-(hydroxymethyl)phenyl, and 2-pyridyl,

or a pharmaceutically acceptable salt thereof.

In another aspect, the invention provides a compound of formula V

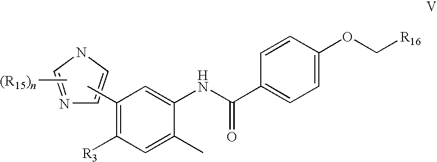

wherein

n is 0, 1, 2, or 3;

R₃ is selected from the group consisting of hydrogen, halogen, and alkyl;

R₁₅ is selected from the group consisting of halogen, hydroxyl, alkyl, alkoxyl, alkoxycarbonyl, sulfinyl, sulfonyl, cyano, cycloalkyl, aryl or a heterocyclyl wherein each R₁₅ is optionally substituted with hydroxyl, halogen, amino, nitro, alkyl, sulfonyl, cyano, alkoxyl or heterocyclyl;

R₁₆ is selected from the group consisting of aryl and heterocyclyl wherein R₁₆ is optionally substituted with R₁₇; and

R₁₇ is selected from the group consisting of halo, alkyl, alkoxy, alkylthio, wherein R₁₇ is optionally substituted with aryl or heteroaryl,

or a pharmaceutically acceptable salt thereof.

In an additional aspect the invention provides a pharmaceutical composition comprising one or more of the compounds described herein, formulated together with one or more pharmaceutically acceptable carriers.

Another aspect of the invention pertains to a method for inhibiting the Hedgehog pathway comprising administering to a subject, e.g., a subject in need thereof, a therapeutically effective amount of one or more of the compounds described herein, or a pharmaceutical composition described herein, such that the Hedgehog pathway is inhibited.

In another aspect, the invention provides a method of reducing cell proliferation, differentiation and/or affecting stromal microenvironment modulation comprising administering to a subject, e.g., a subject in need thereof, a therapeutically effective amount of one or more of the compounds described herein, or a pharmaceutical composition described herein, thereby reducing cell proliferation, differentiation and/or affecting stromal microenvironment modulation in the subject.

### DETAILED DESCRIPTION OF THE INVENTION

The present disclosure relates to heterocyclic amide compounds, which are useful for inhibiting the Hedgehog pathway, and their use in treating a disease or medical condition mediated alone or in part by Hedgehog pathway inhibition. Also disclosed are methods for manufacture of these compounds, pharmaceutical compositions including these compounds, and use of these compounds in the manufacture of medicaments for treating such diseases and medical conditions in a subject.

It is to be understood that both the foregoing general description and the following detailed description are exemplary and explanatory only and are not restrictive of the invention as claimed. Moreover, the present invention, including compounds, methods, and pharmaceutical compositions will be described with reference to the following definitions that, for convenience, are set forth below:

Unless otherwise specified, the chemical groups refer to their unsubstituted and substituted forms.

The term “aldehyde” or “formyl” as used herein refers to the radical —CHO.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, C₂-C₁₀alkenyl, and C₂-C₆alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, etc.

The term “alkoxy” as used herein refers to an alkyl group attached to an oxygen (—O-alkyl-). Exemplary alkoxy groups include, but are not limited to, groups with an alkyl, alkenyl or alkynyl group of 1-12, 1-8, or 1-6 carbon atoms, referred to herein as C₁-C₁₂alkoxy, C₁-C₁₂alkoxy, and C₁-C₆alkoxy, respectively. Exemplary alkoxy groups include, but are not limited to methoxy, ethoxy, etc. Similarly, exemplary “alkenoxy” groups include, but are not limited to vinyloxy, allyloxy, butenoxy, etc.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

Alkyl groups can optionally be substituted with or interrupted by at least one group selected from the group consisting of alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonamide, and sulfonyl.

The term “alkynyl” as used herein refers to an unsaturated straight or branched hydrocarbon having at least one carbon-carbon triple bond, such as a straight or branched group of 2-12, 2-8, or 2-6 carbon atoms, referred to herein as C₂-C₁₂alkynyl, C₂-C₈alkynyl, and C₂-C₆alkynyl, respectively. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl, etc.

The term “amide” or “amido” as used herein refers to a radical of the form —RₐC(O)N(R_{b})—, —RₐC(O)N(R_{b})R_{c}—, or —C(O)NR_{b}R_{c}, wherein R_{b} and R_{c} are each independently selected from the group consisting of alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, and nitro. The amide can be attached to another group through the carbon, the nitrogen, R_{b}, R_{c}, or Rₐ. The amide also may be cyclic, for example R_{b} and R_{c}, Rₐ and R_{b}, or Rₐ and R_{c} may be joined to form a 3- to 12-membered ring, such as a 3- to 10-membered ring or a 5- to 6-membered ring. The term “carboxamido” refers to the structure —C(O)NR_{b}R_{c}.

The term “amidino” as used herein refers to a radical of the form —C(═NR)NR′R″ where R, R′, and R″ can each independently be selected from the group consisting of alkyl, alkenyl, alkynyl, amide, aryl, arylalkyl, cyano, cycloalkyl, haloalkyl, heteroaryl, heterocyclyl, hydroxyl, ketone and nitro.

The term “amine” or “amino” as used herein refers to a radical of the form —NR_{d}Rₑ, —N(R_{d})Rₑ—, or —RₑN(R_{d})R_{f}— where R_{d}, Rₑ, and R_{f} are independently selected from the group consisting of alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, and nitro. The amino can be attached to the parent molecular group through the nitrogen, R_{d}, Rₑ or R_{f}. The amino also may be cyclic, for example any two of Rd, Re or Rf may be joined together or with the N to form a 3- to 12-membered ring, e.g., morpholino or piperidinyl. The term amino also includes the corresponding quaternary ammonium salt of any amino group, e.g., —[N(Rd)(Re)(Rf)]+. Exemplary amino groups include aminoalkyl groups, wherein at least one of R_{d}, Rₑ, or R_{f} is an alkyl group. In specific embodiments, the amino group is a C₁₋₆alkylamino group.

The term “aryl” as used herein refers to a mono-, bi-, or other multi-carbocyclic, aromatic ring system. The aryl group can optionally be fused to one or more rings selected from the group consisting of aryls, cycloalkyls, and heterocyclyls. The aryl groups of this invention can be substituted with groups selected from the group consisting of alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonamide, and sulfonyl. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl.

The term “arylalkyl” as used herein refers to an aryl group having at least one alkyl substituent, e.g. -aryl-alkyl-. Exemplary arylalkyl groups include, but are not limited to, arylalkyls having a monocyclic aromatic ring system, wherein the ring comprises 6 carbon atoms. For example, “phenylalkyl” includes phenylC₄alkyl, benzyl, 1-phenylethyl, 2-phenylethyl, etc.

The term “carbamate” as used herein refers to a radical of the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently selected from the group consisting of alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonyl, and sulfonamide. Exemplary carbamates include, but are not limited to, arylcarbamates or heteroaryl carbamates, e.g., wherein at least one of R_{g}, Rₕ and Rᵢ are independently selected from the group consisting of aryl or heteroaryl, such as phenyl and pyridinyl.

The term “carbonyl” as used herein refers to the radical —C(O)—.

The term “carboxamido” as used herein refers to the radical —C(O)NRR′, where R and R′ may be the same or different. R and R′ may be selected from the group consisting of, for example, alkyl, aryl, arylalkyl, cycloalkyl, formyl, haloalkyl, heteroaryl and heterocyclyl.

The term “carboxy” as used herein refers to the radical —COOH or its corresponding salts, e.g. —COONa, etc.

The term “cyano” as used herein refers to the radical —CN.

The term “cycloalkoxy” as used herein refers to a cycloalkyl group attached to an oxygen.

The term “cycloalkyl” as used herein refers to a monovalent saturated or unsaturated cyclic, bicyclic, or bridged bicyclic hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclohexenes, cyclopentanes, cyclopentenes, cyclobutanes and cyclopropanes. Cycloalkyl groups may be substituted with alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonamide, and sulfonyl. Cycloalkyl groups can be fused to other cycloalkyl, aryl, or heterocyclyl groups.

The term “ether” refers to a radical having the structure —RₗO—Rₘ—, where Rₗ and Rₘ can independently be alkyl, aryl, cycloalkyl, heterocyclyl, or ether. The ether can be attached to the parent molecular group through Rₗ or Rₘ. Exemplary ethers include, but are not limited to, alkoxyalkyl and alkoxyaryl groups. Ether also includes polyethers, e.g., where one or both of Rₗ and Rₘ are ethers.

The terms “halo” or “halogen” or “Hal” as used herein refer to F, Cl, Br, or I.

The term “haloalkyl” as used herein refers to an alkyl group substituted with one or more halogen atoms.

The term “heteroaryl” as used herein refers to a mono-, bi-, or other multi-cyclic, aromatic ring system containing one or more heteroatoms, for example 1 to 4 heteroatoms, such as nitrogen, oxygen, and sulfur. Heteroaryls can be substituted with one or more substituents including alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonamide, and sulfonyl. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2 to 5 carbon atoms and 1 to 3 heteroatoms.

The terms “heterocycle,” “heterocyclyl,” or “heterocyclic” as used herein refer to a saturated, partially unsaturated, or unsaturated 4-12 membered ring containing at least one heteroatom independently selected from the group consisting of nitrogen, oxygen, and sulfur. Unless otherwise specified, the heteroatom may be carbon or nitrogen linked, a —CH₂— group can optionally be replaced by a —C(O)—, and a ring sulfur atom may be optionally oxidized to form a sulfinyl or sulfonyl group. Heterocycles can be aromatic (heteroaryls) or non-aromatic. Heterocycles can be substituted with one or more substituents including alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, hydroxyalkyl, ketone, nitro, sulfide, sulfonamide, and sulfonyl. In certain embodiments, the heterocycles are substituted with a methyl or hydroxyethyl.

Heterocycles also include bicyclic, tricyclic, and tetracyclic groups in which any of the above heterocyclic rings is fused to one or two rings independently selected from the group consisting of aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl. In certain embodiments, the heterocycle is aromatic. In certain other embodiments, the heterocycle is partially or fully saturated. In particular embodiments, the heterocycle is imidazolyl.

The term “heterocyclylalkoxy” as used herein refers to a heterocyclyl attached to an alkoxy group.

The term “heterocyclyloxyalkyl” refers to a heterocyclyl attached to an oxygen (—O—), which is attached to an alkyl group.

The terms “hydroxy” and “hydroxyl” as used herein refers to the radical —OH.

The term “hydroxyalkyl” as used herein refers to a hydroxy radical attached to an alkyl group.

The term “imidazolyl,” as used herein, is art-recognized and includes all isomeric forms of substituted or unsubstituted imidazolyl. For example, the term “imidazolyl” includes 1-imidazolyl, 2-imidazolyl, 3-imidazolyl, 4-imidazolyl, and 5-imidazolyl, each of which may be substituted by 1 to 3 substituents. Such substituents may include halogen, e.g., F, hydroxyl, alkyl, e.g., methyl, alkoxyl, alkoxycarbonyl, sulfinyl, sulfonyl, cyano, cycloalkyl, aryl or a heterocycle.

The term “nitro” as used herein refers to the radical —NO₂.

The term “phenyl” as used herein refers to a 6-membered carbocyclic aromatic ring. The phenyl group can also be fused to a cyclohexane or cyclopentane ring. Phenyl can be substituted with one or more substituents including alkoxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, sulfide, sulfonamide, and sulfonyl.

The term “sulfonamide” as used herein refers to a radical having the structure —N(Rᵣ)—S(O)₂—R_{S}— or —S(O)₂—N(Rᵣ)R_{S}, where Rᵣ, and R_{S} can be, for example, hydrogen, alkyl, aryl, cycloalkyl, and heterocyclyl. Exemplary sulfonamides include alkylsulfonamides (e.g., where R_{S} is alkyl), arylsulfonamides (e.g., where R_{S} is aryl), cycloalkyl sulfonamides (e.g., where R_{S} is cycloalkyl), and heterocyclyl sulfonamides (e.g., where R_{S} is heterocyclyl), etc.

The term “sulfonyl” as used herein refers to a radical having the structure RᵤSO₂—, where Rᵤ can be alkyl, aryl, cycloalkyl, and heterocyclyl, e.g., alkylsulfonyl. The term “alkylsulfonyl” as used herein refers to an alkyl group attached to a sulfonyl group.

The term “sulfide” as used herein refers to the radical having the structure R_{Z}S—, where R_{Z} can be alkoxy, alkyl, alkenyl, alkynyl, amide, amino, aryl, arylalkyl, carbamate, carboxy, cycloalkyl, ester, ether, formyl, haloalkyl, heteroaryl, heterocyclyl, and ketone. The term “alkylsulfide” as used herein refers to an alkyl group attached to a sulfur atom. Exemplary sulfides include “thio,” which as used herein refers to an —SH radical.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The term “subject” is intended to include organisms, e.g., prokaryotes and eukaryotes, which are capable of suffering from proliferative disorders, e.g., cancer, and which are mediated alone or in part by the Hedgehog pathway. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from cancer. In certain embodiments the subject possesses a PTCH loss-of function phenotype, a SMO gain-of-function phenotype or a Hedgehog gain-of-function phenotype.

The compounds of the disclosure may contain one or more chiral centers (e.g., some of which may be explicitly designated as such by the inclusion of bond orientation/designation) and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well known asymmetric synthetic methods.

Geometric isomers can also exist in the compounds of the present invention. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds of the invention can exist in solvated as well as unsolvated forms such as, for example, hydrated forms. In one embodiment, the compound is amorphous. In one embodiment, the compound is a polymorph. In another embodiment, the compound is in a crystalline form.

### I. Compounds of the Invention

Disclosed herein are compounds of formula I or IA

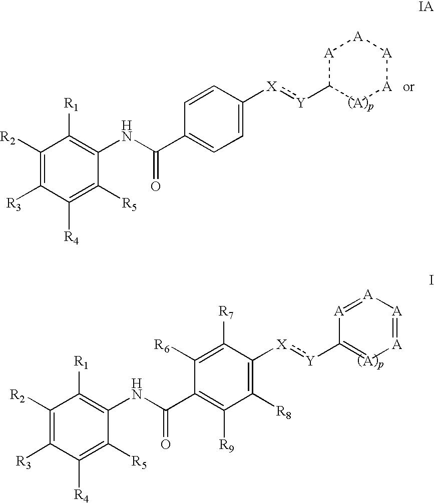

wherein
[symbol] represents a single bond or a double bond;
[symbol] represents a single bond, a double bond, a triple bond, or when X or Y is a direct bond [symbol] represents the absence of a bond;

R₁, R₂, R₃, and R₄ are each independently selected from the group consisting of hydrogen, C₁₋₆alkoxy, C₁₋₆alkoxyC₁₋₆alkyl, C₁₋₆alkyl, aminoC₁₋₆alkyl, C₃₋₈cycloalkyl, cyano, haloC₁₋₆alkyl, halogen, hydroxy, sulfonyl, sulfide, and thio,

with the proviso that either R₂ or R₃ is Z;

each W is independently selected from the group consisting of CR₁₀, NR₁₀, N, O, and S, where R₁₀ is selected from the group consisting of hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, C₃₋₆cycloalkyl, hydroxy, hydroxyC₁₋₆alkyl, nitro, sulfide, sulfonamido, and sulfonyl, or

two adjacent W atoms can be taken together with their R₁₀ substituents to form a fused second ring, wherein the second ring is selected from the group consisting of aryl, C₃₋₈cycloalkyl, a 5- or 6-membered heteroaryl, and a 5- or 6-membered heterocyclyl;

q is 0 or 1, where

if q is 0 and two adjacent W atoms taken together with their R₁₀ substituents form a bicycle selected from the group consisting of benzimidazolyl, benzoxazolyl, benzothiazolyl, and oxazolopyridyl, then at least one A is N,

if q is 1, two W are N, and two adjacent W atoms taken together with their R₁₀ substituents form a quinoxalinyl, then at least one A is N, and

if q is 1 and each W is CR₁₀, then two adjacent W atoms are taken together with their R₁₀ substituents to form a second ring selected from the group consisting of a 5- or 6-membered heteroaryl and a 5- or 6-membered heterocyclyl;

R₅ is selected from the group consisting of alkyl, haloC₁₋₆alkyl, and halogen;

R₆, R₇, R₈ and R₉ are each independently selected from the group consisting of hydrogen, C₁₋₆alkyl, amino, C₃₋₈cycloalkyl, C₁₋₆alkoxy, cyano, haloC₁₋₆alkyl, halogen, sulfide, sulfonyl, and sulfonamido;

when joined by a single bond, X and Y are each independently selected from the group consisting of O, S, SO₂, NR₁₁, and CR₁₁R₁₂, or one of X and Y can be a direct bond,

when joined by a double bond, X and Y are each independently CR₁₁, and

when joined by a triple bond, X and Y are each C;

each R₁₁ and R₁₂ are each independently selected from the group consisting of hydrogen, C₁₋₆alkoxy, C₁₋₆alkyl, amino, cyano, haloC₁₋₆alkyl, halogen, and sulfide;

each A is selected from the group consisting of CR₁₃, CR₁₃R₁₃, NR₁₃, N, O, and S;

each R₁₃ is selected from the group consisting of hydrogen, C₁₋₆alkoxy, C₁₋₆alkoxyamino, C₁₋₆alkoxyC₁₋₆alkyl, C₁₋₆alkoxycarbonyl, C₁₋₆alkyl, C₁₋₆alkylamino, amidino, amido, amino, aminoC₁₋₆alkylamino, aryl, aryloxy, carboxamido, C₃₋₈cycloalkyl, C₃₋₈cycloalkylC₁₋₆alkoxy, cyano, haloC₁₋₆alkyl, halogen, heterocyclyl, heterocyclylC₁₋₆alkyl, heterocyclylC₁₋₆alkoxy, hydroxy, hydroxyC₁₋₆alkyl, hydroxyC₁₋₆alkoxy, nitro, sulfide, sulfonamido, and sulfonyl;

p is 0 or 1, where

if p is 0, then two adjacent A atoms can be taken together with their R₁₃ substituents to form a fused second ring, wherein the second ring is selected from the group consisting of aryl, 6-membered heteroaryl and 6-membered heterocyclyl, and

if p is 1, then two adjacent A atoms can be taken together with their R₁₃ substituents to form a fused second ring, wherein the second ring is selected from the group consisting of aryl, 5- or 6-membered heteroaryl and 5- or 6-membered heterocyclyl;

or a pharmaceutically acceptable salt thereof. In certain embodiments, if R₁₁ and R₁₂ are each fluoro, then each of R₁, R₂, R₄, and R₅ is not fluoro.

In certain embodiments, at least one of X and Y is selected from the group consisting of O or NR₁₁, or at least one A is selected from the group consisting of NR₁₃, N, O, and S.

In another embodiment, R₁₀ is selected from the group consisting of hydrogen, C₁₋₆alkoxycarbonyl, C₁₋₆alkyl, C₁₋₆cycloalkyl, C₁₋₆perfluoroalkyl, amino, hydroxyC₁₋₆alkyl, heterocyclylC₁₋₆alkyl, and nitro. In a particular embodiment, R₁₀ is C₁₋₆alkyl, C₁₋₆cycloalkyl, C₁₋₆perfluoroalkyl, or hydroxyC₁₋₆alkyl.

In one embodiment, Z is a 6,6-fused bicyclic heteroaryl having at least one N heteroatom. In another embodiment, Z is a 5,6-fused bicyclic heteroaryl having at least one N heteroatom. In a further embodiment, the compound of formula I comprises a -5,7-diazabicyclo[4.3.0]nona-2,4,8,10-tetraenyl, such as N-[5-(5,7-diazabicyclo[4.3.0]nona-2,4,8,10-tetraen-4-yl)-2-methyl-phenyl]-4-(pyridin-2-ylmethoxy)benzamide and N-[2-methyl-5-(7H-purin-6-yl)phenyl]-4-(pyridin-2-ylmethoxy)benzamide.

In one embodiment, Z is a 6-membered heteroaryl having two N heteroatoms. In another embodiment, the compound of formula I comprises pyrazinyl or a pyridizinyl. A further embodiment provides a compound of formula I selected from the group consisting of N-[5-(5-aminopyrazin-2-yl)-2-methyl-phenyl]-4-(pyridin-2-ylmethoxy)benzamide and N-[5-(6-amino pyridazin-3-yl)-2-methyl-phenyl]-4-(pyridin-2-ylmethoxy)benzamide.

In one embodiment, Z is a 5-membered heteroaryl having at least one N heteroatom, such as an imidzolyl. In a further embodiment, the compound of formula I is selected from the group consisting of N-[5-(1H-imidazol-4-yl)-2-methyl-phenyl]-4-(pyridin-2-ylmethoxy)benzamide, N-[5-(1H-imidazol-2-yl)-2-methyl-phenyl]-4-(pyridin-2-ylmethoxy)benzamide and N-[2-methyl-5-(1-methylimidazol-2-yl)phenyl]-4-(pyridin-2-ylmethoxy)benzamide. Another embodiment provides a compound of formula I wherein Z is a thiazolyl, such as one selected from the group consisting of N-[2-methyl-5-[5-[(4-methylpiperazin-1-yl)methyl]1,3-thiazol-2-yl]phenyl]-4-(pyridin-2-ylmethoxy)benzamide, N-[2-methyl-5-[5-(pyrazol-1-ylmethyl)-1,3-thiazol-2-yl]phenyl]-4-(pyridin-2-ylmethoxy)benzamide, N-[2-methyl-5-[5-(morpholin-4-ylmethyl) 1,3-thiazol-2-yl]phenyl]-4-(pyridin-2-ylmethoxy)benzamide, N-(2-methyl-5-1,3-thiazol-2-yl-phenyl)-4-(pyridin-2-ylmethoxy)benzamide, and ethyl 4-methyl-2-[4-methyl-3-[[4-(pyridin-2-ylmethoxy)benzoyl]amino]phenyl]1,3-thiazole-5-carboxylate.

In one embodiment, R₂ is Z. In another embodiment, R₃ is Z. In one embodiment, R₁, R₂, R₃, and R₄ are each hydrogen. In one embodiment, R₅ is methyl. In another embodiment, R₆, R₇, R₈ and R₉ are each hydrogen. In a further embodiment, X is O and Y is CH₂.

In another embodiment, at least one A is N and p is 1, for example, a pyridyl. In one embodiment, at least one A is a heteroatom and p is 0.

In another embodiment, the invention relates to a compound of formula II

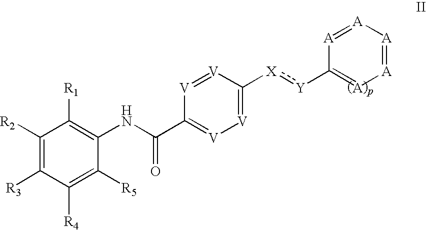

wherein

R₁, R₂, R₃, and R₄ are each independently selected from the group consisting of hydrogen, C₁₋₆alkoxy, C₁₋₆alkoxyC₁₋₆alkyl, C₁₋₆alkyl, aminoC₁₋₆alkyl, C₃₋₈cycloalkyl, cyano, haloC₁₋₆alkyl, halogen, hydroxy, sulfonyl, sulfide, and thio,

with the proviso that either R₂ or R₃ is Z;

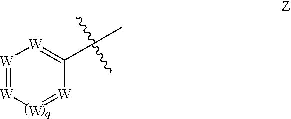

each W is independently selected from the group consisting of CR₁₀, NR₁₀, N, O, and S, where R₁₀ is selected from the group consisting of hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, C₃₋₆cycloalkyl, hydroxy, hydroxyC₁₋₆alkyl, nitro, sulfide, sulfonamido, and sulfonyl, or

two adjacent W atoms can be taken together with their R₁₀ substituents to form a fused second ring, wherein the second ring is selected from the group consisting of aryl, C₃₋₈cycloalkyl, a 5- or 6-membered heteroaryl, and a 5- or 6-membered heterocyclyl;

q is 0 or 1, where

if q is 0 and two adjacent W atoms taken together with their R₁₀ substituents form a bicycle selected from the group consisting of benzimidazolyl, benzoxazolyl, benzothiazolyl, and oxazolopyridyl, then at least one A is N,

if q is 1, two W are N, and two adjacent W atoms taken together with their R₁₀ substituents form a quinoxalinyl, then at least one A is N, and

if q is 1 and each W is CR₁₀, then two adjacent W atoms are taken together with their R₁₀ substituents to form a second ring selected from the group consisting of a 5- or 6-membered heteroaryl and a 5- or 6-membered heterocyclyl;

R₅ is selected from the group consisting of alkyl, haloC₁₋₆alkyl, and halogen;

when joined by a single bond, X and Y are each independently selected from the group consisting of O, S, SO₂, NR₁₁, and CR₁₁R₁₂, or one of X and Y can be a direct bond,

when joined by a double bond, X and Y are each independently CR₁₁, and

when joined by a triple bond, X and Y are each C;

each R₁₁ and R₁₂ are each independently selected from the group consisting of hydrogen, C₁₋₆alkoxy, C₁₋₆alkyl, amino, cyano, haloC₁₋₆alkyl, halogen, and sulfide;

each A is selected from the group consisting of CR₁₃, NR₁₃, N, O, and S;

each R₁₃ is selected from the group consisting of hydrogen, C₁₋₆alkoxy, C₁₋₆alkoxyamino, C₁₋₆alkoxyC₁₋₆alkyl, C₁₋₆alkoxycarbonyl, C₁₋₆alkyl, C₁₋₆alkylamino, amidino, amido, amino, aminoC₁₋₆alkylamino, aryl, aryloxy, carboxamido, C₃₋₈cycloalkyl, C₃₋₈cycloalkylC₁₋₆alkoxy, cyano, haloC₁₋₆alkyl, halogen, heterocyclyl, heterocyclylC₁₋₆alkyl, heterocyclylC₁₋₆alkoxy, hydroxy, hydroxyC₁₋₆alkyl, hydroxyC₁₋₆alkoxy, nitro, sulfide, sulfonamido, and sulfonyl;

each V is independently selected from the group consisting of CR₁₄ and N;

each R₁₄ is selected from the group consisting of hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, hydroxy, hydroxyC₁₋₆alkyl, nitro, sulfide, sulfonamido, and sulfonyl;

p is 0 or 1, where

if p is 0, then two adjacent A atoms can be taken together with their R₁₃ substituents to form a fused second ring, wherein the second ring is selected from the group consisting of aryl, 6-membered heteroaryl and 6-membered heterocyclyl; and

if p is 1, then two adjacent A atoms can be taken together with their R₁₃ substituents to form a fused second ring, wherein the second ring is selected from the group consisting of aryl, 5- or 6-membered heteroaryl and 5- or 6-membered heterocyclyl,

or a pharmaceutically acceptable salt thereof. In certain embodiments, if R₁₁ and R₁₂ are each fluoro, then each of R₁, R₂, R₄, and R₅ is not fluoro.

In further embodiments, the invention relates to a compound of formula III

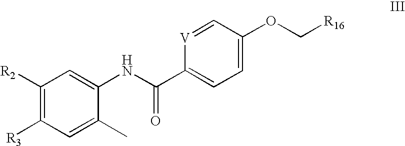

wherein

V is N or CH, e.g., N;

R₂ is selected from the group consisting of pyrazolyl, imidazolyl, benzoimidazol, thiazolyl, pyridyl, triazolyl, purinyl, and quinoxalinyl, wherein R₂ is optionally substituted with one or more R₁₅;

R₁₅ may be selected from the group consisting of alkyl, nitro, aryl, heteroaryl wherein

R₁₅ may be optionally substituted with halo, alkyl, alkoxy, alkylthio, aryl, and heteroaryl;

R₃ is selected from the group consisting of hydrogen and alkyl;

R₁₆ is selected from the group consisting of aryl and heterocyclyl wherein R₁₆ is optionally substituted with R₁₇; and

R₁₇ is selected from the group consisting of halo, alkyl, alkoxy, alkylthio, wherein R₁₇ is optionally substituted with aryl or heteroaryl,

or a pharmaceutically acceptable salt thereof. In certain embodiments, one of R₂ or R₃ is imidazolyl. In certain embodiments, R₁₆ is pyridyl or phenyl.

In another embodiment, the invention relates to a compound of formula IV

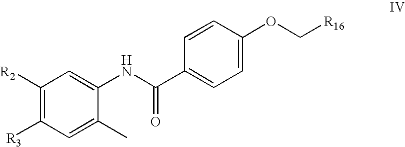

wherein

R₂ is selected from the group consisting of thiazol-2-yl, quinoxalin-2-yl, phenyl, benzothiazol-2-yl, 7H-purin-6-yl, 6-aminopyridazin-3-yl, 6-amino-2-pyridyl, 5-nitro-1H-benzoimidazol-2-yl, 5-methyl-3H-imidazol-4-yl, 5-methyl-1H-imidazol-4-yl, 5-methyl-1,3,4-oxadiazol-2-yl, 5-methyl-1,2,4-oxadiazol-3-yl, 5-ethoxycarbonyl-4-methyl-thiazol-2-yl, 5-aminopyrazin-2-yl, 5-amino-2-pyridyl, 5-[(4-methylpiperazin-1-yl)methyl]thiazol-2-yl, 5,7-diazabicyclo[4.3.0]nona-2,4,8,10-tetraen-4-yl, 5-(trifluoromethyl)-2H-pyrazol-3-yl, 5-(pyrazol-1-ylmethyl)thiazol-2-yl, 5-(morpholinomethyl)thiazol-2-yl, 5-(hydroxymethyl)-1-methyl-imidazol-4-yl, 4-thiazol-2-yl-1H-imidazol-2-yl, 4-thia-1,6-diazabicyclo[3.3.0]octa-2,5,7-trien-7-yl, 4-tert-butyl-1H-imidazol-2-yl, 4-pyridyl, 4-phenyl-1H-imidazol-2-yl, 4-methyl-3H-imidazol-2-yl, 4-methyl-1H-imidazol-2-yl, 4-ethyl-1H-imidazol-2-yl, 4-cyclopropyl-1H-imidazol-2-yl, 4,5-dimethyl-1,2,4-triazol-3-yl, 4-(trifluoromethyl)-3H-imidazol-2-yl, 4-(hydroxymethyl)-1H-imidazol-2-yl, 4-(4-pyrrolidin-1-ylphenyl)-1H-imidazol-2-yl, 4-(3-pyridyl)-1H-imidazol-2-yl, 3-pyridyl, 3-methylimidazol-4-yl, 2-pyridyl, 2-methylpyrazol-3-yl, 2-methyl-1H-imidazol-4-yl, 2,4-dimethylthiazol-5-yl, 2,3-dimethylimidazol-4-yl, 1-methylpyrazol-4-yl, 1-methylimidazol-4-yl, 1-methylimidazol-2-yl, 1-methyl-5-(methylaminomethyl)imidazol-4-yl, 1-isobutylpyrazol-4-yl, 1H-triazol-4-yl, 1H-imidazol-4-yl, 1H-imidazol-2-yl, 1H-benzoimidazol-2-yl, 1-[(3-bromo-2-pyridyl)methyl]imidazol-2-yl, 1,5-dimethylimidazol-2-yl, 1,4-dimethylimidazol-2-yl, 1,3,5-trimethylpyrazol-4-yl, 1,2-dimethylimidazol-4-yl;

R₃ is selected from the group consisting of hydrogen, methyl, and 1H-benzoimidazol-2-yl; and

R₁₆ is selected from the group consisting of 2-cyanophenyl, 2-methoxyphenyl, 3,4-dimethoxy-2-pyridyl, 3,5-dimethoxyphenyl, 3-cyanophenyl, 3-methoxyphenyl, 4-fluorophenyl, 4-methylsulfonylphenyl, 6-chlorobenzo[1,3]dioxol-5-yl, 2-(trifluoromethyl)phenyl, 3-(2-morpholinoethoxy)phenyl, 4-(hydroxymethyl)phenyl, and 2-pyridyl,

or a pharmaceutically acceptable salt thereof. In certain embodiments, R₂ of Formula IV is not one or more of the following: pyridyl, quinoxalin-2-yl, or 1H-benzoimidazol-2-yl.

In another embodiment, the invention provides a compound of formula V

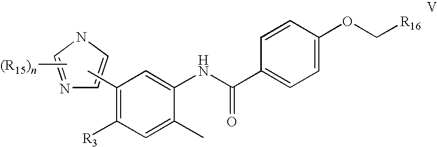

wherein

n is 0, 1, 2, or 3;

R₃ is selected from the group consisting of hydrogen, halogen, e.g., Cl, and alkyl, e.g., methyl;

R₁₅ is selected from the group consisting of halogen, hydroxyl, alkyl, alkoxyl, alkoxycarbonyl, sulfinyl, sulfonyl, cyano, cycloalkyl, aryl or a heterocyclyl wherein each R₁₅ is optionally substituted with hydroxyl, halogen, amino, nitro, alkyl, sulfonyl, cyano, alkoxyl or heterocyclyl;

R₁₆ is selected from the group consisting of aryl and heterocyclyl wherein R₁₆ is optionally substituted with R₁₇; and

R₁₇ is selected from the group consisting of halo, alkyl, alkoxy, alkylthio, wherein R₁₇ is optionally substituted with aryl or heteroaryl,

or a pharmaceutically acceptable salt thereof. In certain embodiments, R₁₅ is halogen, e.g., F, optionally substituted alkyl, e.g., methyl, hydroxylmethyl, methylaminomethyl, aryl, e.g., phenyl, heterocyclyl, or cycloalkyl, e.g., cyclopropyl. In a particular embodiment, n is 0, i.e., R₁₅ is absent. In another particular embodiment, n is 1-3. In a specific embodiment, the imidazolyl moiety is a 5-imidazolyl. In another specific embodiment, the imidazolyl moiety is a 2-imidazolyl. In another specific embodiment, the imidazolyl moiety is a 4-imidazolyl. In certain embodiments, R₁₆ is pyridyl, e.g., 2-pyridyl.

Compounds and compositions of the invention are also useful in the manufacture of a medicament for inhibiting the Hedgehog pathway in a subject in need thereof. One embodiment provides for the use of disclosed compounds and compositions in the manufacture of a medicament for reducing cell differentiation, proliferation, and/or affecting stromal microenvironment modulation in a subject in need thereof. Another embodiment provides for the use of disclosed compounds and compositions in the manufacture of a medicament for treating a disease or medical condition mediated alone or in part by Hedgehog pathway inhibition in a subject in need thereof.

A. Additional Compounds of the Invention

Disclosed herein are compounds of formula VI

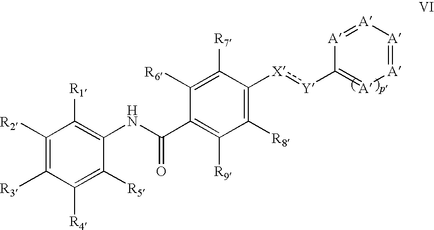

wherein

R_{1′}, R_{2′}, R_{3′}, and R_{4′} are each independently selected from hydrogen, C₁₋₆alkoxy, C₁₋₆alkoxyC₁₋₆alkyl, C₁₋₆alkyl, aminoC₁₋₆alkyl, C₃₋₈cycloalkyl, cyano, haloC₁₋₆alkyl, halogen, hydroxy, sulfonyl, sulfide, and thio,

with the proviso that either R_{2′} or R_{3′} is Z′;

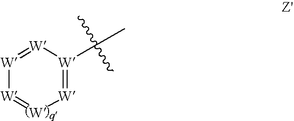

each W′ is independently selected from CR_{10′}, NR_{10′}, N, O, and S, where R_{10′}, is selected from hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, hydroxy, hydroxyC₁₋₆alkyl, nitro, sulfide, sulfonamido, and sulfonyl, or

two adjacent W′ atoms can be taken together to form a fused second ring, wherein the second ring is selected from aryl, C₃₋₈cycloalkyl, a 5- or 6-membered heteroaryl, and a 5- or 6-membered heterocyclyl;

q′ is 0 or 1, where

if q′ is 0 and two adjacent W atoms taken together form a bicycle selected from benzimidazolyl, benzoxazolyl, benzothiazolyl, and oxazolopyridyl, then at least one A′ is N,

if q′ is 1, two W′ are N, and two adjacent W′ atoms taken together form a quinoxalinyl, then at least one A′ is N, and

if q′ is 1 and each W′ is CR₁₀, then two adjacent W atoms are taken together to form a second ring selected from a 5- or 6-membered heteroaryl and a 5- or 6-membered heterocyclyl;

R_{5′} is selected from alkyl, haloC₁₋₆alkyl, and halogen;

R_{6′}, R_{7′}, R_{8′} and R_{9′} are each independently selected from hydrogen, C₁₋₆alkyl, amino, C₃₋₈cycloalkyl, C₁₋₆alkoxy, cyano, haloC₁₋₆alkyl, halogen, sulfide, sulfonyl, and sulfonamido;

when joined by a single bond, X′ and Y′ are each independently selected from O, S, SO₂, NR₁₁, and CR_{11′}R_{12′}, or one of X′ and Y′ can be a direct bond,

when joined by a double bond, X′ and Y′ are each independently CR_{11′}, and

when joined by a triple bond, X′ and Y′ are each C;

each R_{11′}, and R_{12′} are each independently selected from hydrogen, C₁₋₆alkoxy, C₁₋₆alkyl, amino, cyano, haloC₁₋₆alkyl, halogen, and sulfide,

each A′ is selected from CR_{13′}, NR_{13′}, N, O, and S;

R_{13′} is selected from hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, C₃₋₈cycloalkyl, cyano, haloC₁₋₆alkyl, halogen, heterocyclylC₁₋₆alkyl, hydroxy, hydroxyC₁₋₆alkyl, nitro, sulfide, sulfonamido, and sulfonyl;

p′ is 0 or 1, where

if p′ is 0, then two adjacent A′ atoms can be taken together to form a fused second ring, wherein the second ring is selected from aryl, 6-membered heteroaryl and 6-membered heterocyclyl, and

if p′ is 1, then two adjacent A′ atoms can be taken together to form a fused second ring, wherein the second ring is selected from aryl, 5- or 6-membered heteroaryl and 5- or 6-membered heterocyclyl;

wherein if R_{11′} and R_{12′} are each fluoro, then each of R_{1′}, R_{2′}, R_{4′}, and R_{5′} is not fluoro;

and pharmaceutically acceptable salts thereof.

In one embodiment, at least one of X′ and Y′ is selected from O or NR_{11′}, or at least one A′ is selected from NR_{13′}, N, O, and S.

In another embodiment, R_{10′}, is selected from hydrogen, C₁₋₆alkoxycarbonyl, C₁₋₆alkyl, amino, heterocyclylC₁₋₆alkyl, and nitro.

In one embodiment, Z′ is a 6,6-fused bicyclic heteroaryl having at least one N heteroatom. In another embodiment, Z′ is a 5,6-fused bicyclic heteroaryl having at least one N heteroatom. In a further embodiment, the compound of formula I comprises a -5,7-diazabicyclo[4.3.0]nona-2,4,8,10-tetraenyl, such as N-[5-(5,7-diazabicyclo[4.3.0]nona-2,4,8,10-tetraen-4-yl)-2-methyl-phenyl]-4-(pyridin-2-ylmethoxy)benzamide and N-[2-methyl-5-(7H-purin-6-yl)phenyl]-4-(pyridin-2-ylmethoxy)benzamide.

In one embodiment, Z′ is a 6-membered heteroaryl having two N heteroatoms. In another embodiment, the compound of formula I comprises pyrazinyl or a pyridizinyl. A further embodiment provides a compound of formula I selected from N-[5-(5-aminopyrazin-2-yl)-2-methyl-phenyl]-4-(pyridin-2-ylmethoxy)benzamide and N-[5-(6-amino pyridazin-3-yl)-2-methyl-phenyl]-4-(pyridin-2-ylmethoxy)benzamide.

In one embodiment, Z′ is a 5-membered heteroaryl having at least one N heteroatom, such as an imidazolyl. In a further embodiment, the compound of formula I is selected from N-[5-(1H-imidazol-4-yl)-2-methyl-phenyl]-4-(pyridin-2-ylmethoxy)benzamide, N-[5-(1H-imidazol-2-yl)-2-methyl-phenyl]-4-(pyridin-2-ylmethoxy)benzamide and N-[2-methyl-5-(1-methylimidazol-2-yl)phenyl]-4-(pyridin-2-ylmethoxy)benzamide. Another embodiment provides a compound of formula I wherein Z′ is a thiazolyl, such as one selected from N-[2-methyl-5-[5-[(4-methylpiperazin-1-yl)methyl]1,3-thiazol-2-yl]phenyl]-4-(pyridin-2-ylmethoxy)benzamide, N-[2-methyl-5-[5-(pyrazol-1-ylmethyl)-1,3-thiazol-2-yl]phenyl]-4-(pyridin-2-ylmethoxy)benzamide, N-[2-methyl-5-[5-(morpholin-4-ylmethyl)1,3-thiazol-2-yl]phenyl]-4-(pyridin-2-ylmethoxy)benzamide, N-(2-methyl-5-1,3-thiazol-2-yl-phenyl)-4-(pyridin-2-ylmethoxy)benzamide, and ethyl 4-methyl-2-[4-methyl-3-[[4-(pyridin-2-ylmethoxy)benzoyl]amino]phenyl]1,3-thiazole-5-carboxylate.

In one embodiment, R_{2′} is Z′. In another embodiment, R_{3′} is Z′. In one embodiment, R_{1′}, R_{2′}, R_{3′}, and R_{4′} are each hydrogen. In one embodiment, R_{5′} is methyl. In another embodiment, R_{6′}, R_{7′}, R_{8′} and R_{9′} are each hydrogen. In a further embodiment, X′ is O and Y is CH₂.

In another embodiment, at least one A′ is N and p′ is 1, for example, a pyridyl. In one embodiment, at least one A′ is a heteroatom and p′ is 0.

In another embodiments, the invention relates to a compound of formula VII

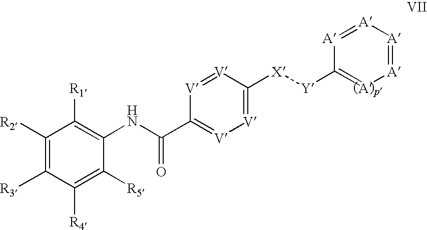

or pharmaceutically acceptable salts thereof wherein,

each V′ is independently selected from CR_{14′} and N;

R_{1′}, R_{2′}, R_{3′}, and R_{4′} are each independently selected from hydrogen, C₁₋₆alkoxy, C₁₋₆alkoxyC₁₋₆alkyl, C₁₋₆alkyl, aminoC₁₋₆alkyl, C₃₋₈cycloalkyl, cyano, haloC₁₋₆alkyl, halogen, hydroxy, sulfonyl, sulfide, and thio,

with the proviso that either R_{2′} or R_{3′} is Z′;

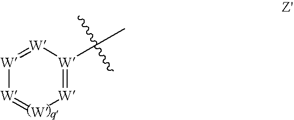

each W′ is independently selected from CR_{10′}, NR_{10′}, N, O, and S, where R_{10′} is selected from hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, hydroxy, hydroxyC₁₋₆alkyl, nitro, sulfide, sulfonamido, and sulfonyl, or

two adjacent W′ atoms can be taken together to form a fused second ring, wherein the second ring is selected from aryl, C₃₋₈cycloalkyl, a 5- or 6-membered heteroaryl, and a 5- or 6-membered heterocyclyl;

q′ is 0 or 1, where

if q′ is 0 and two adjacent W′ atoms taken together form a bicycle selected from benzimidazolyl, benzoxazolyl, benzothiazolyl, and oxazolopyridyl, then at least one A′ is N,

if q′ is 1, two W′ are N, and two adjacent W′ atoms taken together form a quinoxalinyl, then at least one A′ is N, and

if q′ is 1 and each W′ is CR_{10′}, then two adjacent W′ atoms are taken together to form a second ring selected from a 5- or 6-membered heteroaryl and a 5- or 6-membered heterocyclyl;

R_{5′} is selected from alkyl, haloC₁₋₆alkyl, and halogen;

when joined by a single bond, X′ and Y′ are each independently selected from O, S, SO₂, NR_{11′}, and CR_{11′}R_{12′}, or one of X′ and Y′ can be a direct bond,

when joined by a double bond, X′ and Y′ are each independently CR_{11′}, and

when joined by a triple bond, X′ and Y′ are each C;

each R_{11′} and R_{12′} are each independently selected from hydrogen, C₁₋₆alkoxy, C₁₋₆alkyl, amino, cyano, haloC₁₋₆alkyl, halogen, and sulfide,

each A′ is selected from CR_{13′}, NR_{13′}, N, O, and S;

each R_{13′} is selected from hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, C₃₋₈cycloalkyl, cyano, haloC₁₋₆alkyl, halogen, heterocyclylC₁₋₆alkyl, hydroxy, hydroxyC₁₋₆alkyl, nitro, sulfide, sulfonamido, and sulfonyl;

R_{14′} is selected from hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, hydroxy, hydroxyC₁₋₆alkyl, nitro, sulfide, sulfonamido, and sulfonyl;

p′ is 0 or 1, where

if p′ is 0, then two adjacent A′ atoms can be taken together to form a fused second ring, wherein the second ring is selected from aryl, 6-membered heteroaryl and 6-membered heterocyclyl, and

if p′ is 1, then two adjacent A′ atoms can be taken together to form a fused second ring, wherein the second ring is selected from aryl, 5- or 6-membered heteroaryl and 5- or 6-membered heterocyclyl; and

wherein if R_{11′} and R_{12′} are each fluoro, then each of R_{1′}, R_{2′}, R_{4′}, and R_{5′} is not fluoro.

In further embodiments, the invention relates to a compound of formula VIII

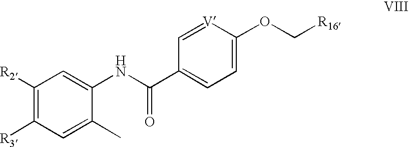

or pharmaceutically acceptable salts thereof wherein,

V′ is N or CH;

R_{2′} is selected from pyrazolyl, imidazolyl, benzoimidazol, thiazolyl, pyridyl, triazolyl, purinyl, and quinoxalinyl; wherein R_{2′} is optionally substituted with one or more R_{15′};

R_{15′} may be selected from alkyl, nitro, aryl, heteroaryl wherein R₁₅ may be optionally substituted with halo, alkyl, alkoxy, alkylthio, aryl, and heteroaryl;

R_{3′} is selected from hydrogen, methyl, and 1H-benzoimidazol-2-yl; and

R_{16′} is selected from aryl and heterocyclyl wherein R_{16′} is optionally substituted with R₁₆;

R_{17′} is selected from halo, alkyl, alkoxy, alkylthio, wherein R_{17′} is optionally substituted with aryl or heteroaryl.

In another embodiment, the invention relates to a compound of formula IX

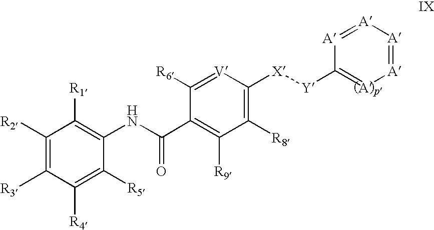

or pharmaceutically acceptable salts thereof, wherein, V′ is selected from N and CH;

R_{1′}, R_{2′}, R_{3′}, and R_{4′}, are each independently selected from hydrogen, C₁₋₆alkoxy, C₁₋₆alkoxyC₁₋₆alkyl, C₁₋₆alkyl, aminoC₁₋₆alkyl, C₃₋₈cycloalkyl, cyano, haloC₁₋₆alkyl, halogen, hydroxy, sulfonyl, sulfide, and thio,

with the proviso that either R_{2′} or R_{3′} is Z′;

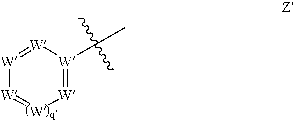

each W′ is independently selected from CR_{10′}, NR_{10′}, N, O, and S, where R_{10′}, is selected from hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, hydroxy, hydroxyC₁₋₆alkyl, nitro, sulfide, sulfonamido, and sulfonyl, or

two adjacent W′ atoms can be taken together to form a fused second ring, wherein the second ring is selected from aryl, C₃₋₈cycloalkyl, a 5- or 6-membered heteroaryl, and a 5- or 6-membered heterocyclyl;

q′ is 0 or 1, where

if q′ is 0 and two adjacent W′ atoms taken together form a bicycle selected from benzimidazolyl, benzoxazolyl, benzothiazolyl, and oxazolopyridyl, then at least one A′ is N,

if q′ is 1, two W′ are N, and two adjacent W′ atoms taken together form a quinoxalinyl, then at least one A′ is N, and

if q′ is 1 and each W′ is CR₁₀, then two adjacent W′ atoms are taken together to form a second ring selected from a 5- or 6-membered heteroaryl and a 5- or 6-membered heterocyclyl;

R_{5′} is selected from alkyl, haloC₁₋₆alkyl, and halogen;

R_{6′}, R_{8′} and R_{9′} are each independently selected from hydrogen, C₁₋₆alkyl, amino, C₃₋₈cycloalkyl, C₁₋₆alkoxy, cyano, haloC₁₋₆alkyl, halogen, sulfide, sulfonyl, and sulfonamido;

when joined by a single bond, X′ and Y′ are each independently selected from O, S, SO₂, NR_{11′}, and CR_{11′}R_{12′}, or one of X′ and Y′ can be a direct bond,

when joined by a double bond, X′ and Y′ are each independently CR_{11′}, and

when joined by a triple bond, X′ and Y′ are each C;

each R_{11′}, and R_{12′} are each independently selected from hydrogen, C₁₋₆alkoxy, C₁₋₆alkyl, amino, cyano, haloC₁₋₆alkyl, halogen, and sulfide,

each A′ is selected from CR_{13′}, NR₁₃, N, O, and S;

each R_{13′} is selected from hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, C₃₋₈cycloalkyl, cyano, haloC₁₋₆alkyl, halogen, heterocyclylC₁₋₆alkyl, hydroxy, hydroxyC₁₋₆alkyl, nitro, sulfide, sulfonamido, and sulfonyl;

p′ is 0 or 1, where

if p′ is 0, then two adjacent A′ atoms can be taken together to form a fused second ring, wherein the second ring is selected from aryl, 6-membered heteroaryl and 6-membered heterocyclyl, and

if p′ is 1, then two adjacent A′ atoms can be taken together to form a fused second ring, wherein the second ring is selected from aryl, 5- or 6-membered heteroaryl and 5- or 6-membered heterocyclyl;

wherein if R_{11′} and R_{12′} are each fluoro, then each of R_{1′}, R_{2′}, R_{4′}, and R_{5′} is not fluoro;

and pharmaceutically acceptable salts thereof.

In another embodiment, the invention relates to a compound of formula X

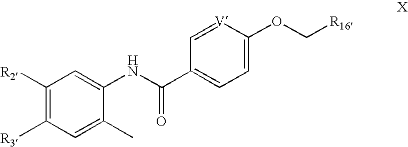

or pharmaceutically acceptable salts thereof wherein,

V′ is N or CH;

R_{2′} is selected from 1,3,5-trimethylpyrazol-4-yl, 1,4-dimethylimidazol-2-yl, 1,5-dimethylimidazol-2-yl, 1H-benzoimidazol-2-yl, 1H-imidazol-2-yl, 1H-imidazol-4-yl, 1-isobutylpyrazol-4-yl, 1-methylimidazol-2-yl, 1-methylimidazol-4-yl, 1-methylpyrazol-4-yl, 2,3-dimethylimidazol-4-yl, 2,4-dimethylthiazol-5-yl, 2-methylpyrazol-3-yl, 2-pyridyl, 3-methylimidazol-4-yl, 3-pyridyl, 4,5-dimethyl-1,2,4-triazol-3-yl, 4-methyl-1H-imidazol-2-yl, 4-pyridyl, 4-thia-1,6-diazabicyclo[3.3.0]octa-2,5,7-trien-7-yl, 5-(morpholinomethyl)thiazol-2-yl, 5-(pyrazol-1-ylmethyl)thiazol-2-yl, 5-(trifluoromethyl)-2H-pyrazol-3-yl, 5,7-diazabicyclo[4.3.0]nona-2,4,8,10-tetraen-4-yl, 5-[(4-methylpiperazin-1-yl)methyl]thiazol-2-yl, 5-amino-2-pyridyl, 5-aminopyrazin-2-yl, 5-ethoxycarbonyl-4-methyl-thiazol-2-yl, 5-methyl-1,2,4-oxadiazol-3-yl, 5-methyl-1,3,4-oxadiazol-2-yl, 5-methyl-1H-imidazol-4-yl, 5-nitro-1H-benzoimidazol-2-yl, 6-amino-2-pyridyl, 6-aminopyridazin-3-yl, 7H-purin-6-yl, benzothiazol-2-yl, phenyl, quinoxalin-2-yl, and thiazol-2-yl;

R_{3′} is selected from hydrogen, methyl, and 1H-benzoimidazol-2-yl; and

R_{16′} is selected from 2-cyanophenyl, 2-methoxyphenyl, 3,4-dimethoxy-2-pyridyl, 3,5-dimethoxyphenyl, 3-cyanophenyl, 3-methoxyphenyl, 4-fluorophenyl, 4-methylsulfonylphenyl, 6-chlorobenzo[1,3]dioxol-5-yl, 2-(trifluoromethyl)phenyl, 3-(2-morpholinoethoxy)phenyl, 4-(hydroxymethyl)phenyl, and 2-pyridyl.

B. Synthetic Schemes

In addition, compounds of formula I (or formula IA) can be synthesized from the general synthetic methods described below in Schemes 1-5. It is to be understood that compounds of formula I, such as those synthesized according to the general methods below, may themselves be further derivatized to form other compounds of formula I. The following schemes are meant to be exemplary only, and one of ordinary skill in the art would recognize viable combinations thereof.

Anilines or phenols (X═O, N) of compound 1 (Scheme IA) can be alkylated using standard conditions by reaction with electrophilic benzylic compounds such as halide or tosylate 2 in the presence of a base, such as sodium hydride or potassium carbonate. Hydrolysis of the corresponding ester 3 using standard conditions, such as aqueous ethanol and sodium hydroxide, results in carboxylic acid 4. Amide bond formation is achieved by reaction of 4 and aniline 5 in the presence of a coupling/dehydrating agent such as, for example, HATU or EDCI and optionally a tertiary base such as diisopropylethylamine or N-methylmorpholine. Alternatively, acid 4 can be converted to an activated acid chloride or acid anhydride with reagents such as thionyl chloride or isopropyl chloroformate, respectively, and then further reacted with aniline 5 using similar tertiary organic bases. The resultant arylboronate 6 can be reductively added to an aryl or heteroaryl halide or triflate such as 7 using transition metal mediated transformations such as, for example, Suzuki couplings with Pd(0) species, e.g. Pd(PPh₃)₄ and Cs₂CO₃. Resulting compound I-A corresponds to a compound of Formula I wherein Y═CR11R12. Using a similar synthetic sequence, but employing alternate aniline 8 in the amide coupling step with compound 4 (as in Scheme 1A) yields compounds of Formula I-B (Scheme 1B).

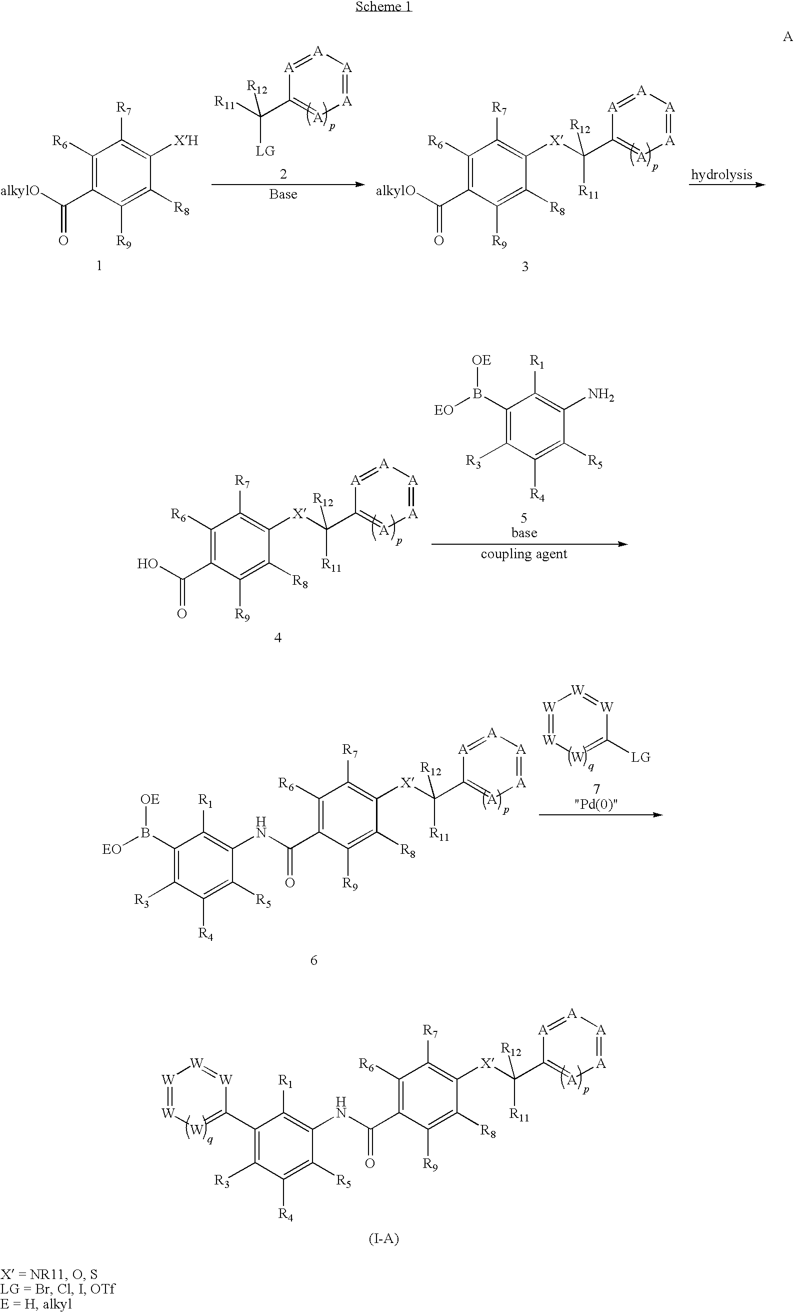

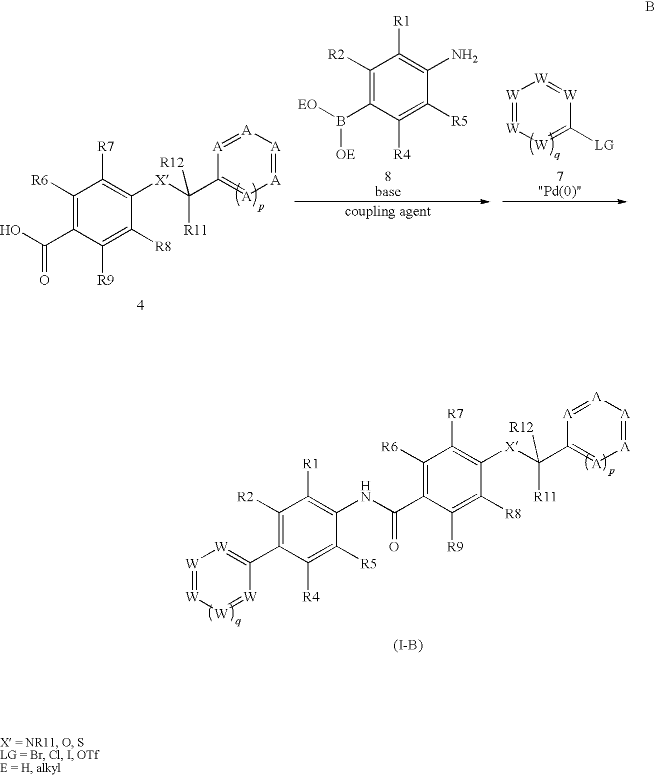

Compounds of Formula IA can also be synthesized utilizing the alternate sequence outlined in Scheme 2, where the last step in the synthesis is a transition metal mediated Suzuki or Negishi coupling between electrophile 9 and boronate or organozinc 10. Aryl or heterocycle 9 can be synthesized from compound 4 by reaction with the appropriate carboxylic acid derivative in the same fashion shown in Scheme 1A.

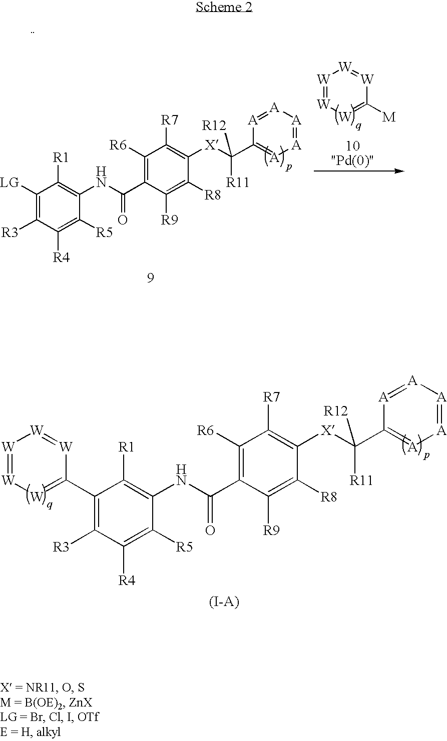

In addition, compounds of Formula (I) can be synthesized from a variety of other methods (Scheme 3) utilizing aryl alkynes 11, nitriles 12, or aldehydes/ketones/acids 13 as starting points to the Z ring of Formula (I). For example, alkynes are useful precursors to rings such as, for example, triazoles (Bock et al. Eur. J. Org. Chem. 51-68 (2006)) and pyrazoles (Fulton et al. Eur. J. Org. Chem. 1479-1492 (2005)) by reaction with azido and diazo reagents, respectively. Nitriles are useful as starting materials to thiazoles and other heterocycles (Collier, S. J.; Langer, P., Science of Synthesis, 19:411 (2004)). Aldehydes and ketones can be used as precursors to a variety of heterocycles (Nakamura, et al., J. Med. Chem. 46:5416-5427 (2003)) including, but not limited to, imidazoles, benzimidazoles, and quinoxalines. Carboxylic acids and derivatives thereof can be converted to a variety of heterocycles such as, for example, benzimidazoles or benzothiazoles.

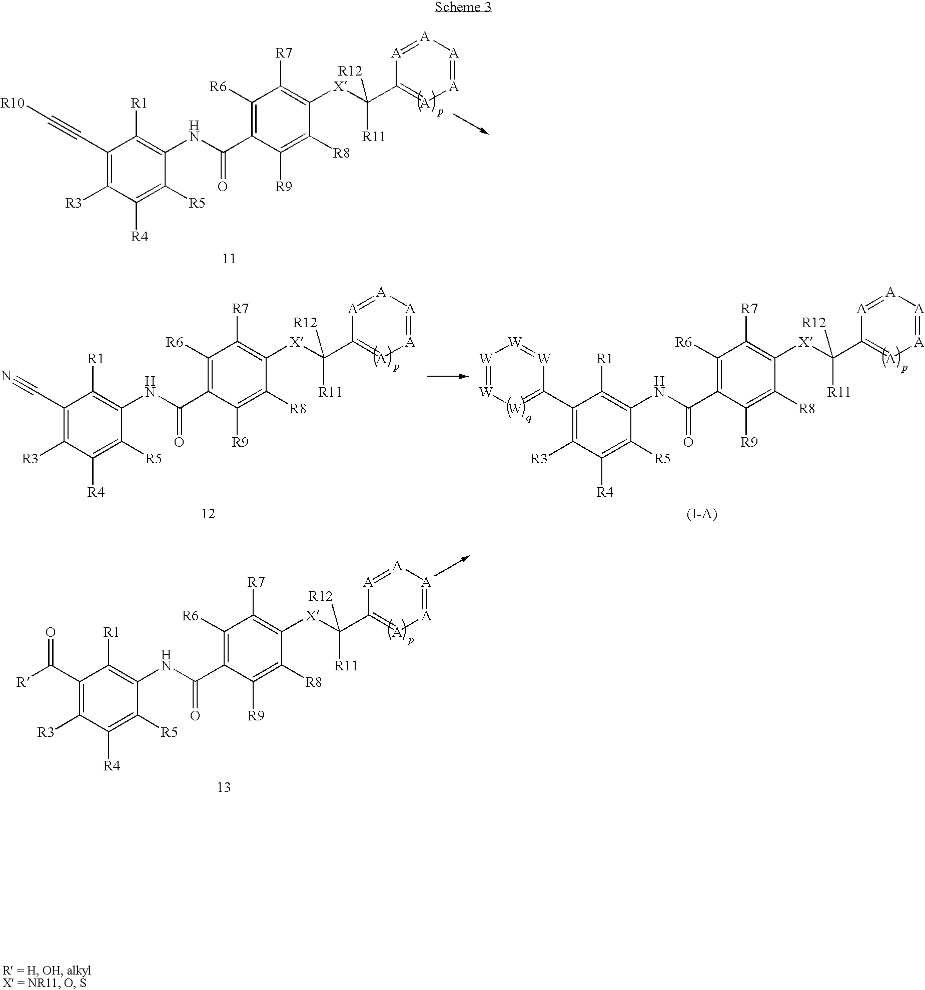

As shown in Scheme 4, when X and Y are both CR11R12, a subset of compounds of Formula (I), (I-C), can be synthesized from compound 14, which can be constructed from methods already described above in Schemes 1-3. Coupling of aniline 14 with acid 15, using standard amide bond formation reaction conditions as described in Scheme I, is followed by a palladium(0)-mediated Heck-type coupling of the resultant compound 16 to alkene 17. This coupling yields the unsaturated compound of Formula I-C. Further reduction of 1-C using methods such as catalytic hydrogenation gives saturated compounds of Formula I-D. Compounds of Formula I-C or I-D can also be constructed via alkyne intermediates synthesized by Sonogashira coupling with alkyne 18 to yield compound 19 that can be then further reduced to either I-C or I-D (Scheme 4B).

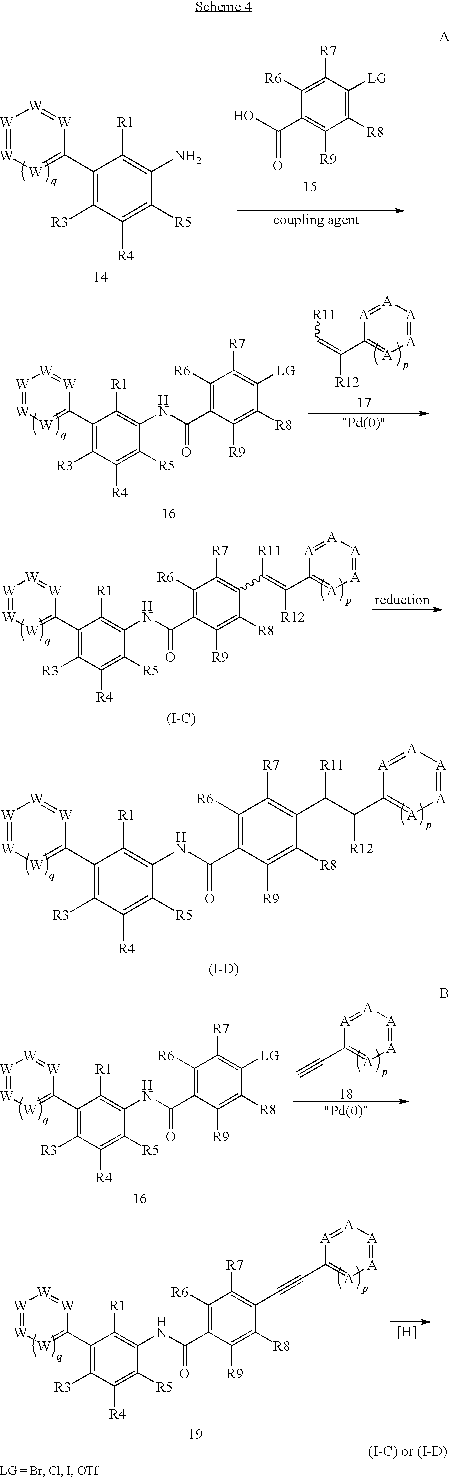

Compounds of Formula I-E can be constructed utilizing the synthetic route outlined in Scheme 5. Reductive amination of aldehyde or ketone 20 with aniline 21 using reagents such as sodium borohydride yields benzylic amine 22. Subjecting 22 to hydrolysis conditions as described in Scheme 1 then leads to acid 23, which can be coupled to aniline 5 as described in Scheme 1 to form amide 24. Transition metal mediated coupling to aryl or heteroaryl derivative 7 using conditions described in Scheme 1 results in the formation of compounds of Formula I-E.

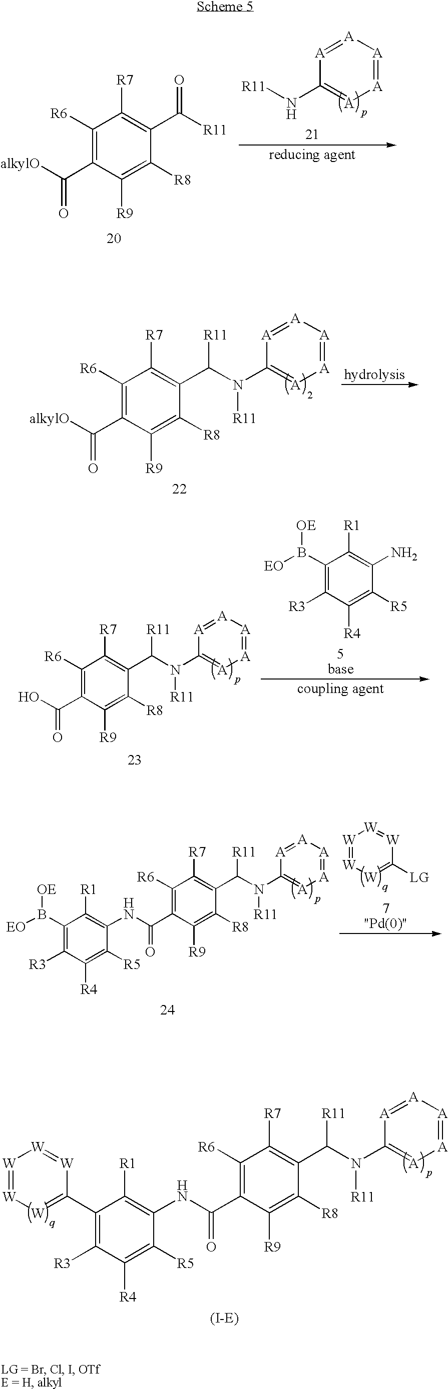

The synthesis of compounds of Formula I-F is shown in Scheme 6. Hydride reduction of ketone or aldehyde 20 using reagents such as sodium borohydride (R12=H) yields alcohol 25. Alternatively, organometallic addition to 18 using Grignard or organolithium reagents R₁₂-M yields alcohol 25. Mitsunobo reaction of 25 with alcohol 26 yields ether 27. Alternatively, alcohol 25 can be converted to an intermediate halide (using reagents such as PX₃) or other leaving group such as a mesylate by reaction with mesyl chloride and base. Subsequent alkylation of alcohol 26 with 25 can be effected with a variety of bases such as sodium hydride to give 27. Compounds of Formula I-F can be obtained utilizing the series of transformations of compound 27 as are described for compound 22 in Scheme 5.

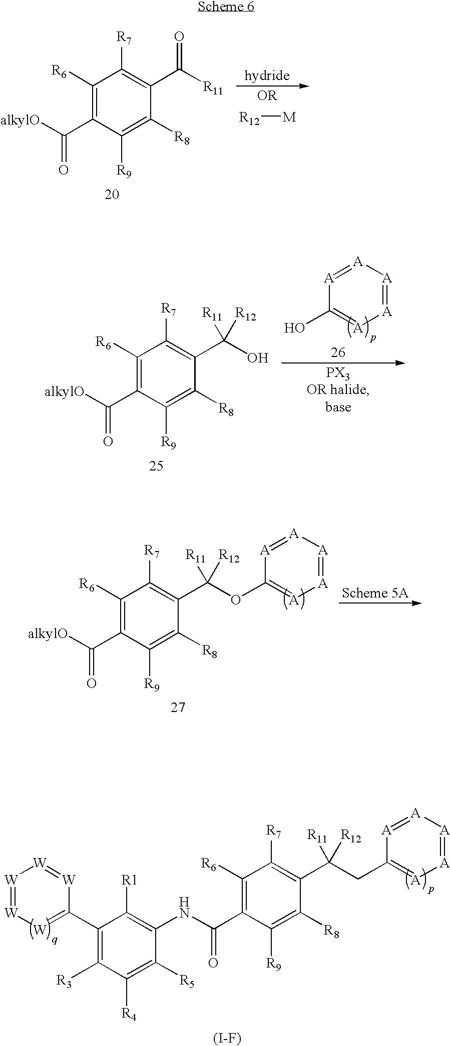

### II. Pharmaceutical Compositions

The present disclosure also provides pharmaceutical compositions comprising compounds as disclosed herein formulated together with one or more pharmaceutically acceptable carriers. These formulations include those suitable for oral, rectal, topical, buccal and parenteral (e.g., subcutaneous, intramuscular, intradermal, or intravenous) administration, although the most suitable form of administration in any given case will depend on the degree and severity of the condition being treated and on the nature of the particular compound being used.

In one embodiment, the compound or pharmaceutical composition is administered to a subject such as a warm-blooded animal. In another embodiment, the warm-blooded animal is a mammal, such as a human.

Formulations suitable for oral administration may be presented in discrete units, such as capsules, cachets, lozenges, or tablets, each containing a predetermined amount of the compound as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association the active compound and the carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with the compound as a unit-dose formulation, for example, a tablet, which may contain from about 0.05% to about 95% by weight of the active compound. Other pharmacologically active substances may also be present, including other compounds. The formulations of the invention may be prepared by any of the well known techniques of pharmacy involving admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the active compound with a liquid or finely divided solid carrier, or both, and then, if necessary, shaping the product. For example, a tablet may be prepared by compressing or molding a powder or granules of the compound, optionally with one or more accessory ingredients. Compressed tablets may be prepared by compressing, in a suitable machine, the compound in a free-flowing form, such as a powder or granules optionally mixed with a binder, lubricant, inert diluent and/or surface active/dispersing agent(s). Molded tablets may be made by molding, in a suitable machine, the powdered compound moistened with an inert liquid diluent.

Formulations suitable for buccal (sub-lingual) administration include lozenges comprising a compound in a flavored base, usually sucrose and acacia or tragacanth, and pastilles comprising the compound in an inert base such as gelatin and glycerin or sucrose and acacia.

Formulations of the present invention suitable for parenteral administration comprise sterile aqueous preparations of the compounds, which are approximately isotonic with the blood of the intended recipient. These preparations are administered intravenously, although administration may also be effected by means of subcutaneous, intramuscular, or intradermal injection. Such preparations may conveniently be prepared by admixing the compound with water and rendering the resulting solution sterile and isotonic with the blood. Injectable compositions according to the invention may contain from about 0.1 to about 5% w/w of the active compound.

Formulations suitable for rectal administration are presented as unit-dose suppositories. These may be prepared by admixing the compound with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound is generally present at a concentration of from about 0.1% to about 15% w/w of the composition, for example, from about 0.5% to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 10 μg to about 100 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. Compounds of the invention may be administered in a dose of about 1 μg/kg to about 200 mg/kg daily; such as from about 1 μg/kg to about 150 mg/kg, from about 1 mg/kg to about 200 mg/kg, from about 1 μg/kg to about 100 mg/kg, from about 1 μg/kg to about 1 mg/kg, from about 50 μg/kg to about 200 mg/kg, from about 10 μg/kg to about 1 mg/kg, from about 10 μg/kg to about 100 μg/kg, from about 100 μg to about 10 mg/kg, and from about 500 μg/kg to about 50 mg/kg. The dosages, however, may be varied depending upon the requirements of the patient, the severity of the condition being treated, and the compound being used. In one embodiment, the therapeutically effective amount of a disclosed compound is sufficient to establish a maximal plasma concentration ranging from about 0.001 μM to about 100 μM, e.g., from about 1 μM to about 20 μM. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture assays or animal models. Levels in plasma may be measured, for example, by high performance liquid chromatography. The effects of any particular dosage can be monitored by a suitable bioassay. Examples of dosages are: about 0.1×IC₅₀, about 0.5×IC₅₀, about 1×IC₅₀, about 5×IC₅₀, 10×IC₅₀, about 50×IC₅₀, and about 100×IC₅₀.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50(4):219-244 (1966) and Table 1 for Equivalent Surface Area Dosage Factors).

TABLE 1

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | 1/2 | 1/4 | 1/6 | 1/12 |
| Rat | 2 | 1 | 1/2 | 1/4 | 1/7 |
| Monkey | 4 | 2 | 1 | 3/5 | 1/3 |
| Dog | 6 | 4 | 3/5 | 1 | 1/2 |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and sex, as well as the severity of the medical condition in the subject. The dosage may be determined by a physician and adjusted, as necessary, to suit observed effects of the treatment.

One embodiment provides administration of a compound of formula Ito a subject in conjunction with radiation treatment. In another embodiment, a compound as disclosed herein, or a pharmaceutically acceptable salt or hydrate thereof, is administered in combination with one or more therapeutic agents. The therapeutic agent can be administered separately, sequentially or simultaneously with the compound disclosed herein. Dosage ranges for combination therapies may be commensurate with that of monotherapy.

The therapeutic agent(s) can provide additive or synergistic value relative to the administration of the compound alone. The therapeutic agent can be, for example, selected from the group consisting of:

(i) antiproliferative/antineoplastic drugs and combinations thereof, as used in medical oncology, such as alkylating agents (for example, cis-platin, carboplatin, cyclophosphamide, nitrogen mustard, melphalan, chlorambucil, busulphan and nitrosoureas); antimetabolites (for example, antifolates such as fluoropyrimidines (like 5-fluorouracil and tegafur), raltitrexed, methotrexate, cytosine arabinoside and hydroxyurea); antitumor antibiotics (for example, anthracyclines like adriamycin, bleomycin, doxorubicin, daunomycin, epirubicin, idarubicin, mitomycin-C, dactinomycin and mithramycin); antimitotic agents (for example, vinca alkaloids like vincristine, vinblastine, vindesine and vinorelbine and taxoids like taxol and taxotere); and topoisomerase inhibitors (for example, epipodophyllotoxins like etoposide and teniposide, amsacrine, topotecan and camptothecin);

(ii) cytostatic agents such as antiestrogens (for example, tamoxifen, toremifene, raloxifene, droloxifene and iodoxyfene), estrogen receptor down regulators (for example, fulvestrant), antiandrogens (for example, bicalutamide, flutamide, nilutamide and cyproterone acetate), LHRH antagonists or LHRH agonists (for example, goserelin, leuprorelin and buserelin), progestogens (for example, megestrol acetate), aromatase inhibitors (for example, anastrozole, letrozole, vorazole and exemestane) and inhibitors of 5α-reductase such as finasteride;

(iii) agents which inhibit cancer cell invasion (for example, metalloproteinase inhibitors like marimastat and inhibitors of urokinase plasminogen activator receptor function);

(iv) inhibitors of growth factor function: for example, such inhibitors include growth factor antibodies, growth factor receptor antibodies (for example, the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, inhibitors of the epidermal growth factor family (for example, EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), inhibitors of the platelet-derived growth factor family and inhibitors of the hepatocyte growth factor family;

(v) antiangiogenic agents such as those which inhibit the effects of vascular endothelial growth factor, (for example, the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example, linomide, inhibitors of integrin αvβ3 function and angiostatin);

(vi) vascular damaging agents such as Combretastatin A4 and compounds disclosed in International Patent Applications WO 99/02166, WO00/40529, WO 00/41669, WO01/92224, WO02/04434 and WO02/08213;

(vii) antisense therapies, for example, those which are directed to the targets listed above, such as ISIS 2503, an anti-ras antisense;

(viii) gene therapy approaches, including for example, approaches to replace aberrant genes such as aberrant p53 or aberrant BRCA1 or BRCA2, GDEPT (gene-directed enzyme pro-drug therapy), approaches such as those using cytosine deaminase, thymidine kinase or a bacterial nitroreductase enzyme and approaches to increase patient tolerance to chemotherapy or radiotherapy such as multi-drug resistance gene therapy;

(ix) immunotherapy approaches, including for example ex vivo and in vivo approaches to increase the immunogenicity of patient tumor cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumor cell lines and approaches using anti-idiotypic antibodies;

(x) cell cycle inhibitors, including for example, CDK inhibitiors (e.g., flavopiridol) and other inhibitors of cell cycle checkpoints (e.g., checkpoint kinase); inhibitors of aurora kinase and other kinases involved in mitosis and cytokinesis regulation (e.g., mitotic kinesins); and histone deacetylase inhibitors; and

(xi) endothelin antagonists, including endothelin A antagonists, endothelin B antagonists and endothelin A and B antagonists; for example ZD4054 and ZD1611 (WO 96/40681), atrasentan and YM598.

Compounds of formula I can be useful as pharmaceutical tools in the development and standardization of in vitro and in vivo test systems for evaluating the effects of Hedgehog pathway inhibition in laboratory animals such as cats, dogs, rabbits, monkeys, rats and mice, as part of the search for new therapeutic agents.

### III. Methods of Use

In certain embodiments, the compounds and compositions of the invention can be useful in methods for inhibiting the Hedgehog pathway. Disclosed herein are methods for reducing cell differentiation, proliferation, and/or affecting stromal microenvironment modulation comprising administering a therapeutically effective amount of a compound of the invention to a subject in need thereof. Inhibiting the Hedgehog pathway provides useful methods for treating diseases or medical conditions mediated alone or in part by this pathway. These diseases include cancer and other proliferative diseases.

While the primary focus has been on cancer, Hedgehog antagonists have also been shown to reduce the symptoms of psoriasis (Tas et al. Dermatology 209:126-131 (2004)). Psoriasis is a chronic skin disorder typically characterized by skin lesions and plaques, and is currently understood to be autoimmune disease, though its etiology is not well defined. As such, compounds of the invention are expected to have a beneficial effect on subjects having psoriasis.

Accordingly, one embodiment provides a method for inhibiting the Hedgehog pathway comprising administering to a subject in need thereof a therapeutically effective amount of a disclosed compound or pharmaceutical composition. Another embodiment provides a method of reducing cell proliferation, differentiation and/or affecting stromal microenvironment modulation comprising administering to a subject in need thereof a therapeutically effective amount of a disclosed compound or pharmaceutical composition. In one embodiment, the cell is a stromal cell. In another embodiment, the cell is a cancer cell. In a further embodiment, the cell is a stem cell, such as a cancer stem cell.

In one embodiment, stromal microenvironment modulation comprises vascular modulation. In another embodiment, stromal microenvironment modulation comprises downregulation of the Hedgehog pathway in stromal cells. In a further embodiment, the stromal cell is a fibroblast.

In one embodiment, cell proliferation, differentiation and/or stromal microenvironment modulation are prevented by administering to a subject in need thereof a therapeutically effective amount of a disclosed compound or pharmaceutical composition. As used herein, “prevention” or “preventing” refers to a reduction of the risk of acquiring a given disease or disorder.

Also disclosed are methods for treating a disease or medical condition mediated alone or in part by Hedgehog pathway inhibition comprising administering to a subject in need thereof a therapeutically effective amount of a compound or composition as disclosed herein.

In one embodiment, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

In one embodiment, the disease or medical condition mediated alone or in part by Hedgehog pathway inhibition is associated with cancer. Exemplary diseases and conditions include, but are not limited to, basal cell carcinoma, medulloblastoma, rhabdomyosarcoma, sarcoma, lymphoma, leukemia, glioblastoma, cancers of the prostate, pancreas, ovary, melanoma, breast, colon, lung, esophagus, gastric, biliary, hepatocellular and multiple myeloma. Thus, compounds and compositions of the invention possess anti-proliferative activity, such as anti-cancer activity.

In another embodiment, the disease or medical condition is psoriasis. In a further embodiment, psoriasis can be treated by administering a compound of the invention in combination with one or more anti-psoriasis agents.

in one embodiment, the subject is characterized as having a phenotype selected from the group consisting of a PTCH loss-of function phenotype, a SMO gain-of-function phenotype, and a Hedgehog gain-of-function phenotype.

### EXEMPLIFICATION

The following descriptions of experiments, procedures, examples, and intermediates are intended to exemplify embodiments of the invention, and are in no way intended to be limiting.

The compounds of the present invention can be prepared in a number of ways well known to one skilled in the art of organic synthesis. More specifically, compounds of the invention may be prepared using the reactions and techniques described herein. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated.

In addition, the compounds listed herein below are intended to be individual and separate embodiments, and any substitution depicted in these compounds are intended to be separately identifiable as a particular substitution applicable to the genus structures described herein, e.g., Formulae I-IV.

The starting materials for the examples are either commercially available or are readily prepared by standard methods from known materials. In the following examples, the conditions are as follows, unless stated otherwise:
(i) temperatures are given in degrees Celsius (° C.); operations are carried out at room temperature (RT) or ambient temperature, such as a range of about 18-25° C., unless otherwise stated;
(ii) solutions are dried over anhydrous sodium sulfate or magnesium sulfate, for example; evaporation organic of organic solvent is carried out using a rotary evaporator under reduced pressure (e.g., about 4.5-30 mmHg) with a bath temperature of, for example, up to about 60° C.;
(iii) chromatography refers to flash chromatography on silica gel; thin layer chromatography (TLC) was carried out on silica gel plates;
(iv) in general, the course of reactions was followed by TLC or liquid chromatography/mass spectroscopy (LC/MS), and reaction times are given for illustration only;
(v) final products have been analyzed using proton nuclear magnetic resonance (NMR) spectra and/or mass spectra data;
(vi) yields are given for illustration only and are not necessarily those that can be obtained by diligent process development; preparations can be repeated if more material is desired;
(vii) when given, nuclear magnetic resonance (NMR) data is in the form of delta (6) values for major diagnostic protons, given in part per million (ppm) relative to tetramethylsilane (TMS) as an internal standard, determined at either 300 or 400 MHz in d₆-DMSO;
(viii) chemical symbols have their usual meanings in the art;
(ix) solvent ratio is given in volume:volume (v/v) terms;
(x) purification of the compounds can be carried out using one or more of the following methods:
   a) flash chromatography on normal-phase silica gel;
   b) flash chromatography on silica gel using Isco Combiflash® separation system: RediSep normal phase flash column at a flow rate such as 20-80 mL/min (ISCO MPLC);
   c) flash chromatography on silica gel using Biotage separation system;
   d) Gilson semiprep HPLC separation system: for example, YMC pack ODS-AQ column, 100×20 mm, S 5 μm 12 nm, water (0.1% trifluoroacetic acid) and MeCN (0.1% trifluoroacetic acid), or water (10 mM NH₄OAc with 5% MeCN) and MeCN as solvents, 10-20 min run; and
   e) crystallization or recrystallization using standard techniques.

Abbreviations used herein denote the following compounds, reagents and substituents: ammonium acetate (NH₄OAc), acetonitrile (MeCN), O-(7-azabenzotriazol-1-yl)-N,N,N′,N′-tetramethyluronium hexafluorophosphate (HATU), N,N-diisopropylethylamine or Hunig's Base (DIPEA), triethylamine (TEA), dimethylacetamide (DMA), ethylene glycol dimethyl ether (DME), diethyl ether (Et₂O), dimethylformamide (DMF), dimethylsulfoxide (DMSO), ethanol (EtOH), methanol (MeOH), tetrahydrofuran (THF), N-(3-dimethylaminopropyl)-N-ethylcarbodiimide (EDCI), fetal bovine serum (FBS), 1-hydroxy-7-azabenzotriazole (HOAt), Sonic Hedgehog (shh ligand), para-nitrophenol (pNp), phosphate-buffered saline (PBS), methylene chloride or CH₂Cl₂ (DCM), ethyl acetate (EtOAc), sodium sulfate (Na₂SO₄), magnesium sulfate (MgSO₄), sodium hydroxide (NaOH), lithium hydroxide (LiOH), hydrogen chloride (HCl), hydrogen (H₂), cesium carbonate (Cs₂CO₃), potassium carbonate (K₂CO₃), sodium carbonate (Na₂CO₃), sodium bicarbonate (NaHCO₃), potassium bicarbonate (KHCO₃), tetrakis(triphenylphosphine) palladium (0) [Pd(PPh₃)₄], ammonium chloride (NH₄Cl), sodium borohydride (NaBH₄), N,N-dimethylpyridin-4-amine (DMAP), ammonium hydroxide (NH₄OH), 1,2-dichloroethane (DCE), potassium acetate (KOAc), N-methylpyrrolidinone (NMP), acetic acid (AcOH), methyl-tent-butyl ether (MTBE), diisopropyl azodicarboxylate (DIAD), 2,2′-bis(diphenylphosphino)-1,1′-binaphthyl (BINAP), tris(dibenzyldeneacetone)dipalladium (Pd₂dba₃), [1,1′-bis(diphenylphosphino)ferrocene]dichloropalladium(II) [PdCl₂(dppf)], sodium hydride (NaH), and sodium iodide (NaI).

### Example 1

### N-[5-(1H-imidazol-4-yl)-2-methyl-phenyl]-4-(pyridin-2-ylmethoxy)benzamide

### 1a. Ethyl 4-(pyridin-2-ylmethoxy)benzoate

To a solution of ethyl 4-hydroxybenzoate (15 g, 90.27 mmol) in dry DMA (300 mL) was added K₂CO₃ (31.2 g, 225.67 mmol) and 2-(chloromethyl)pyridine hydrochloride (29.6 g, 180.53 mmol) portion-wise. The solution was stirred at RT for 7 days. The reaction was poured into 900 mL of sat. NaHCO₃ while stirring. The precipitate was filtered off, washed with water (500 mL), then 1:1 hexanes:Et₂O (400 mL). The resulting solid was dried first under suction, then in a vacuum oven overnight at 55° C. to yield the title compound as a white solid (26 g, unpurified). A second batch was obtained by refiltration of the filtrate after 1 day. The crystalline product was washed with water, followed by 1:1 hexanes:Et₂O, and dried, first under suction, then in a vacuum oven at 55° C. to yield a further 6.43 g (28%). ¹H NMR (DMSO-d₆) δ 8.58 (d, 1H), 7.90 (d, 2H), 7.83 (td, 1H), 7.51 (d, 1H), 7.35 (dd, 1H), 7.13 (d, 2H), 5.26 (s, 2H), 4.26 (q, 2H), 1.29 (t, 3H). MS (M+H⁺)=258.

### 1b. 4-(Pyridin-2-ylmethoxy)benzoic acid

To a slurry of ethyl 4-(pyridin-2-ylmethoxy)benzoate (26 g, 90.27 mmol, batch 1 from above) in EtOH (230 mL) was added NaOH (2.5M) (51.1 mL, 127.75 mmol) slowly. The reaction was stirred at RT for 48 h. More NaOH (100 mL, 2M) was added and the reaction was stirred overnight again. The reaction mixture was concentrated in vacuo, then diluted to 100 mL with water and acidified to pH˜6 with 10% HCl. The precipitate was collected, washed with water, dried under suction, then washed with Et₂O and dried under suction to yield the title compound (9.35 g, 45%). The filtrate was further acidified with 10% HCl, filtered, washed with water, Et₂O and dried under suction to yield more product (1.617 g, 8%). Combined yield=53%. ¹H NMR (DMSO-d₆) δ 12.65 (s, 1H), 8.58 (d, 1H), 7.88 (d, 2H), 7.83 (td, 1H), 7.51 (d, 1H), 7.35 (dd, 1H), 7.10 (d, 2H), 5.25 (s, 2H). MS (M+H⁺)=230.

### 1c. 4-Methyl-3-(4-(pyridin-2-ylmethoxy)benzamido)phenylboronic acid

To 4-(pyridin-2-ylmethoxy)benzoic acid (300 mg, 1.31 mmol), HATU (522 mg, 1.37 mmol) and DIPEA (0.457 mL, 2.62 mmol) was added DMF (3 mL). After stirring for 1 h at 50° C., the mixture was cooled and 3-amino-4-methylphenylboronic acid (198 mg, 1.31 mmol) was added. The reaction was reheated to 50° C. for 6 h, an additional equivalent of 3-amino-4-methylphenylboronic acid was added, and the reaction was stirred at RT for 48 h. The reaction was poured into sat. NaCl solution (30 mL). The precipitate was filtered, washed with water, followed by Et₂O, and dried under suction to yield the title compound (434 mg, 92%). ¹H NMR (DMSO-d₆) δ 9.75 (s, 1H), 8.61 (d, 1H), 7.96 (d, 2H), 7.90 (td, 1H), 7.67 (s, 1H), 7.58 (m, 2H), 7.40 (dd, 1H), 7.22 (d, 1H), 7.14 (d, 2H), 5.29 (s, 2H), 2.20 (s, 3H). MS (M+H⁺)=363.

### 1d. N-[5-(1H-Imidazol-4-yl)-2-methyl-phenyl]-4-(pyridin-2-ylmethoxy)benzamide

A mixture of 4-methyl-3-(4-(pyridin-2-ylmethoxy)benzamido)phenyl boronic acid (50 mg, 0.14 mmol), Cs₂CO₃ (135 mg, 0.41 mmol), Pd(PPh₃)₄(23.93 mg, 0.02 mmol) and 4-bromo-1H-imidazole (26 mg, 0.18 mmol) was purged with nitrogen before adding degassed dioxane (690 μL) and water (230 μL) and heating in a microwave for 40 min at 150° C. After cooling, the aqueous layer was removed with a pipette, and the organic layer was diluted with DMSO (1 mL) and filtered through a 0.2 μm filter. The filtrate was concentrated to a volume of 1 mL and purified by Gilson HPLC (20-75% MeCN/10 mM NH₄OAc in water). The fractions were concentrated and lyophilized to yield the title compound (19 mg, 0.049 mmol, 35%). ¹H NMR (DMSO-d₆) 12.11 (s, 1H), 9.76 (s, 1H), 8.59 (d, 1H), 7.97 (d, 2H), 7.85 (td, 1H), 7.70 (s, 1H), 7.67 (s, 1H), 7.56 (m, 2H), 7.36 (dd, 1H), 7.21 (d, 1H), 7.15 (d, 2H), 5.27 (s, 2H), 2.18 (s, 3H). MS (M+H⁺)=385.

The following Examples 2-28 were prepared in a similar fashion to Example 1 utilizing commercially available starting materials:

| | | | MS | |
|---|---|---|---|---|
| Ex. | Name | MW | (M + H⁺) | ¹H NMR (δ ppm) |
| 2 | N-[2-methyl-5-[5-[(4- | 513.22 | 514 | 9.83 (s, 1H), 8.59 (d, 1H), 7.97 (d, 2H), |
| | methylpiperazin-1- | | | 7.93 (s, 1H), 7.85 (td, 1H), 7.71 (s, 1H), |
| | yl)methyl]1,3-thiazol-2- | | | 7.67 (dd, 1H), 7.53 (d, 1H), 7.36 (m, 2H), |
| | yl]phenyl]-4-(pyridin-2- | | | 7.16 (d, 2H), 5.28 (s, 2H), 3.73 (s, 2H), |
| | ylmethoxy)benzamide | | | 2.42 (m, 8H), 2.27 (s, 3H), 2.19 (s, 3H) |
| 3 | N-[5-(5,7- | 434.17 | 435 | 11.67 (s, 1H), 9.83 (s, 1H), 8.60 (d, 1H), |
| | diazabicyclo[4.3.0]nona- | | | 8.11 (d, 1H), 8.00 (m, 3H), 7.86 (m, 2H), |
| | 2,4,8,10-tetraen-4-yl)-2- | | | 7.63 (d, 1H), 7.54 (d, 1H), 7.47 (m, 1H), |
| | methyl-phenyl]-4-(pyridin- | | | 7.36 (m, 2H), 7.16 (d, 2H), 6.45 (dd, 1H), |
| | 2-ylmethoxy)benzamide | | | 5.28 (s, 2H), 2.27 (s, 3H) |
| 4 | N-[2-methyl-5-[5-(pyrazol- | 481.16 | 482 | 9.82 (s, 1H), 8.59 (d, 1H), 7.96 (d, 2H), |
| | 1-ylmethyl)1,3-thiazol-2- | | | 7.92 (d, 1H), 7.85 (m, 3H), 7.66 (dd, 1H), |
| | yl]phenyl]-4-(pyridin-2- | | | 7.53 (d, 1H), 7.49 (d, 1H), 7.36 (m, 2H), |
| | ylmethoxy)benzamide | | | 7.16 (d, 2H), 6.27 (t, 1H), 5.62 (s, 2H), |
| | | | | 5.28 (s, 2H), 2.26 (s, 3H) |
| 5 | N-[2-methyl-5-(4-thia-1,6- | 440.13 | 441 | 9.71 (s, 1H), 8.59 (d, 1H), 7.95 (d, 2H), |
| | diazabicyclo[3.3.0]octa- | | | 7.84 (td, 1H), 7.59 (m, 2H), 7.53 (d, 1H), |
| | 2,5,7-trien-7-yl)phenyl]-4- | | | 7.35 (dd, 1H), 7.31 (d, 1H), 7.25 (d, 1H), |
| | (pyridin-2- | | | 7.19 (m, 1H), 7.14 (m, 1H), 7.14 (d, 2H), |
| | ylmethoxy)benzamide | | | 5.27 (s, 2H), 2.21 (s, 3H) |
| 6 | N-[5-(5-aminopyridin-2-yl)- | 410.17 | 411 | 9.94 (s, 1H), 8.64 (d, 1H), 7.98 (m, 5H), |
| | 2-methyl-phenyl]-4- | | | 7.87 (d, 1H), 7.70 (dd, 1H), 7.64 (m, 2H), |
| | (pyridin-2- | | | 7.46 (m, 2H), 7.18 (d, 2H), 5.33 (s, 2H), |
| | ylmethoxy)benzamide | | | 2.30 (s, 3H) |
| 7 | N-(2-methyl-5-pyridin-3-yl- | 395.16 | 396 | 9.96 (s, 1H), 9.18 (d, 1H), 8.82 (d, 1H), |
| | phenyl)-4-(pyridin-2- | | | 8.75 (d, 1H), 8.69 (d, 1H), 8.06 (m, 2H), |
| | ylmethoxy)benzamide | | | 8.01 (d, 2H), 7.87 (d, 1H), 7.69 (m, 2H), |
| | | | | 7.54 (dd, 1H), 7.47 (d, 1H), 7.19 (d, 2H), |
| | | | | 5.37 (s, 2H), 2.30 (s, 3H) |
| 8 | N-[5-(6-aminopyridin-2-yl)- | 410.17 | 411 | 13.97 (br s, 1H), 10.01 (s, 1H), 8.71 (d, |
| | 2-methyl-phenyl]-4- | | | 1H), 8.25 (br s, 2H), 8.11 (t, 1H), 8.02 (d, |
| | (pyridin-2- | | | 2H), 7.96 (dd, 1H), 7.91 (d, 1H), 7.78 (dd, |
| | ylmethoxy)benzamide | | | 1H), 7.75 (d, 1H), 7.59 (m, 1H), 7.49 (d, |
| | | | | 1H), 7.20 (d, 1H), 7.19 (d, 2H), 6.97 (d, |
| | | | | 1H), 5.40 (s, 2H), 2.31 (s, 3H) |
| 9 | N-(2-methyl-5-pyridin-2-yl- | 395.16 | 396 | 9.85 (s, 1H), 8.64 (d, 1H), 8.59 (d, 1H), |
| | phenyl)-4-(pyridin-2- | | | 8.08 (d, 1H), 7.98 (d, 2H), 7.93 (d, 1H), |
| | ylmethoxy)benzamide | | | 7.85 (m, 3H), 7.54 (d, 1H), 7.34 (m, 3H), |
| | | | | 7.16 (d, 2H), 5.28 (s, 2H), 2.27 (s, 3H) |
| 10 | N-[5-(5-aminopyrazin-2- | 411.17 | 412 | 9.79 (s, 1H), 8.59 (d, 1H), 8.46 (d, 1H), |
| | yl)-2-methyl-phenyl]-4- | | | 7.97 (d, 2H), 7.93 (d, 1H), 7.88 (d, 1H), |
| | (pyridin-2- | | | 7.85 (td, 1H), 7.70 (dd, 1H), 7.53 (d, 1H), |
| | ylmethoxy)benzamide | | | 7.36 (dd, 1H), 7.29 (d, 1H), 7.15 (d, 2H), |
| | | | | 6.52 (s, 2H), 5.28 (s, 2H), 2.23 (s, 3H) |
| 11 | N-(2-methyl-5-pyridin-4-yl- | 395.16 | 396 | 10.02 (s, 1H), 8.92 (d, 2H), 8.69 (d, 1H), |
| | phenyl)-4-(pyridin-2- | | | 8.37 (d, 2H), 8.05 (m, 2H), 8.03 (d, 2H), |
| | ylmethoxy)benzamide | | | 7.86 (dd, 1H), 7.70 (d, 1H), 7.54 (m, 2H), |
| | | | | 7.19 (d, 2H), 5.38 (s, 2H), 2.34 (s, 3H) |
| 12 | N-[2-methyl-5-[5- | 500.19 | 501 | 9.84 (s, 1H), 8.59 (d, 1H), 7.97 (d, 2H), |
| | (morpholin-4-ylmethyl)1,3- | | | 7.93 (d, 1H), 7.85 (td, 1H), 7.72 (s, 1H), |
| | thiazol-2-yl]phenyl]-4- | | | 7.68 (d, 1H), 7.53 (d, 1H), 7.36 (m, 2H), |
| | (pyridin-2- | | | 7.16 (d, 2H), 5.28 (s, 2H), 3.73 (s, 2H), |
| | ylmethoxy)benzamide | | | 3.57 (m, 4H), 2.41 (m, 4H), 2.27 (s, 3H) |
| 13 | N-[2-methyl-5-(1- | 398.17 | 399 | 9.82 (s, 1H), 8.59 (d, 1H), 7.97 (d, 2H), |
| | methylimidazol-2- | | | 7.84 (td, 1H), 7.65 (d, 1H), 7.53 (d, 1H), |
| | yl)phenyl]-4-(pyridin-2- | | | 7.46 (dd, 1H), 7.36 (m, 2H), 7.23 (s, 1H), |
| | ylmethoxy)benzamide | | | 7.15 (d, 2H), 6.95 (s, 1H), 5.28 (s, 2H), |
| | | | | 3.74 (s, 3H), 2.27 (s, 3H) |
| 14 | N-[2-methyl-5-(5-nitro-1H- | 479.16 | 480 | 13.59 (s, 1H), 9.92 (s, 1H), 8.60 (d, 1H), |
| | benzoimidazol-2- | | | 8.53 (d, 0.5H), 8.34 (d, 0.5H), 8.26 (d, |
| | yl)phenyl]-4-(pyridin-2- | | | 1H), 8.12 (ddd, 1H), 8.01 (m, 3H), |
| | ylmethoxy)benzamide | | | 7.86 (td, 1H), 7.82 (d, 0.5H), 7.70 (d, 0.5H), |
| | | | | 7.55 (d, 1H), 7.50 (m, 1H), 7.37 (dd, 1H), |
| | | | | 7.18 (d, 2H), 5.29 (s, 2H), 2.33 (s, 3H) |
| 15 | N-[5-(6-aminopyridazin-3- | 411.17 | 412 | 9.84 (s, 1H), 8.59 (d, 1H), 7.98 (d, 2H), |
| | yl)-2-methyl-phenyl]-4- | | | 7.94 (d, 1H), 7.85 (td, 1H), 7.78 (d, 1H), |
| | (pyridin-2- | | | 7.73 (dd, 1H), 7.54 (d, 1H), 7.35 (m, 2H), |
| | ylmethoxy)benzamide | | | 7.15 (d, 2H), 6.83 (d, 1H), 6.46 (s, 2H), |
| | | | | 5.28 (s, 2H), 2.25 (s, 3H) |
| 16 | N-(2-methyl-5-1,3-thiazol- | 401.12 | 402 | 9.86 (s, 1H), 8.59 (d, 1H), 7.97 (m, 3H), |
| | 2-yl-phenyl)-4-(pyridin-2- | | | 7.90 (d, 1H), 7.85 (td, 1H), 7.76 (d, 1H), |
| | ylmethoxy)benzamide | | | 7.73 (dd, 1H), 7.53 (d, 1H), 7.39 (d, 1H), |
| | | | | 7.36 (m, 1H), 7.16 (d, 2H), 5.28 (s, 2H), |
| | | | | 2.27 (s, 3H) |
| 17 | N-(5-benzothiazol-2-yl-2- | 451.14 | 452 | 9.91 (s, 1H), 8.60 (d, 1H), 8.14 (m, 2H), |
| | methyl-phenyl)-4-(pyridin- | | | 8.04 (d, 1H), 8.00 (d, 2H), 7.85 (m, 2H), |
| | 2-ylmethoxy)benzamide | | | 7.54 (m, 2H), 7.46 (m, 2H), 7.36 (dd, 1H), |
| | | | | 7.18 (d, 2H), 5.29 (s, 2H), 2.32 (s, 3H) |
| 18 | N-[2-methyl-5-(7H-purin-6- | 436.16 | 437 | 10.02 (s, 1H), 8.94 (s, 1H), 8.80 (d, 1H), |
| | yl)phenyl]-4-(pyridin-2- | | | 8.74 (d, 1H), 8.66 (m, 2H), 8.15 (m, 1H), |
| | ylmethoxy)benzamide | | | 8.04 (d, 2H), 7.97 (dd, 1H), 7.78 (d, 1H), |
| | | | | 7.62 (m, 1H), 7.49 (d, 1H), 7.20 (d, 2H), |
| | | | | 7.16 (dd, 1H), 5.42 (s, 2H), 2.32 (s, 3H) |
| 19 | ethyl 4-methyl-2-[4-methyl- | 487.16 | 488 | 9.87 (s, 1H), 8.60 (d, 1H), 8.05 (d, 1H), |
| | 3-[[4-(pyridin-2- | | | 7.98 (d, 2H), 7.86 (td, 1H), 7.76 (dd, 1H), |
| | ylmethoxy)benzoyl]amino]phenyl]1, | | | 7.54 (d, 1H), 7.41 (d, 1H), 7.37 (dd, 1H), |
| | 3-thiazole-5- | | | 7.17 (d, 2H), 5.29 (s, 2H), 4.29 (q, 2H), |
| | carboxylate | | | 2.68 (s, 3H), 2.29 (s, 3H), 1.30 (t, 3H) |
| 20 | N-[5-(1,5- | 412.49 | 413 | 2.22 (s, 3 H), 2.28 (s, 3 H), 3.59 (s, 3 H), |
| | dimethylimidazol-2-yl)-2- | | | 5.29 (s, 2 H), 6.75 (s, 1 H), 7.16 (d, 2 H), |
| | methyl-phenyl]-4-(pyridin- | | | 7.37 (m, 3 H), 7.56 (m, 2 H), 7.85 (m, 1 |
| | 2-ylmethoxy)benzamide | | | H), 7.98 (d, 2 H), 8.60 (m, 1 H), 9.82 (s, 1 |
| | | | | H) |
| 21 | N-[2-methyl-5-(1-methyl- | 398.46 | 399 | 2.20 (s, 3 H), 3.68 (s, 3 H), 5.29 (s, 2 H), |
| | 1H-imidazol-4-yl)phenyl]- | | | 7.16 (d, 2 H), 7.23 (d, 1 H), 7.38 (d, 1 H), |
| | 4-(pyridin-2- | | | 7.55 (m, 3 H), 7.61 (s, 1 H), 7.69 (d, 1 H), |
| | ylmethoxy)benzamide | | | 7.87 (m, 1 H), 7.98 (d, 2 H), 8.60 (d, 1 H), |
| | | | | 9.77 (br s, 1 H) |
| 22 | N-(2-methyl-5-quinoxalin- | 446.51 | 447 | 2.33 (s, 3 H), 5.30 (s, 2 H), 7.19 (d, 2 H), |
| | 2-yl-phenyl)-4-(pyridin-2- | | | 7.37 (m, 1 H), 7.53 (dd, 2 H), 7.87 (td, 3 |
| | ylmethoxy)benzamide | | | H), 8.03 (d, 2 H), 8.15 (m, 3 H), 8.34 (d, 1 |
| | | | | H), 8.62 (s, 1 H), 9.59 (s, 1 H), 9.99 (s, 1 |
| | | | | H) |
| 23 | N-[5-(1,2-dimethyl-1H- | 412.49 | 413 | 2.33 (s, 3 H), 2.65 (s, 3 H), 3.68 (s, 3 H), |
| | imidazol-5-yl)-2- | | | 5.37 (s, 2 H), 7.19 (d, 2 H), 7.32 (m, 1 H), |
| | methylphenyl]-4-(pyridin-2- | | | 7.49 (m, 2 H), 7.57 (s, 1 H), 7.68 (d, 1 H), |
| | ylmethoxy)benzamide | | | 7.74 (s, 1 H), 8.01 (d, 3 H), 8.67 (br s, 1 |
| | | | | H), 9.94 (s, 1 H), 14.47 (br s, 1 H) |
| 24 | N-[2-methyl-5-(2-methyl- | 398.46 | 399 | 2.20 (s, 3 H), 2.31 (s, 3 H), 5.28 (s, 2 H), |
| | 1H-imidazol-4-yl)phenyl]- | | | 7.16 (m, 2 H), 7.22 (d, 1 H), 7.37 (m, 2 |
| | 4-(pyridin-2- | | | H), 7.49 (d, 1 H), 7.55 (d, 1 H), 7.65 (s, 1 |
| | ylmethoxy)benzamide | | | H), 7.85 (m, 1 H), 7.98 (m, 2 H), 8.60 (d, |
| | | | | 1 H), 9.76 (s, 1 H), 11.98 (br s, 1 H) |
| 25 | N-[2-methyl-5-(5-methyl- | 398.46 | 399 | 2.18 (br s, 3 H) 2.23 (br s, 3 H) 5.29 (br |
| | 1H-imidazol-2-yl)phenyl]- | | | s, 2 H) 6.79 (br s, 1 H) 7.17 (m, 2 H) |
| | 4-(pyridin-2- | | | 7.30 (d, 1 H) 7.34-7.47 (m, 1 H) 7.55 (d, 1 H) |
| | ylmethoxy)benzamide | | | 7.67 (d, 1 H) 7.86 (br s, 2 H) 7.99 (m, 2 |
| | | | | H) 8.61 (br s, 1 H) 9.82 (br s, 1 H) |
| | | | | 12.18 (br s, 1 H) |
| 26 | N-[5-(1,4-dimethyl-1H- | 412.49 | 413 | 2.11 (s, 3 H) 2.28 (s, 3 H) 3.68 (s, 3 H) |
| | imidazol-2-yl)-2- | | | 5.29 (s, 2 H) 6.91 (s, 1 H) 7.17 (m, 2 H) |
| | methylphenyl]-4-(pyridin-2- | | | 7.35 (t, 2 H) 7.45 (d, 1 H) 7.54 (d, 1 H) |
| | ylmethoxy)benzamide | | | 7.67 (s, 1 H) 7.85 (td, 1 H) 8.00 (m, 2 H) |
| | | | | 8.60 (d, 1 H) 9.84 (s, 1 H) |
| 27 | N-[2-methyl-5-(4-methyl- | 398.46 | 399 | 2.22 (s, 3 H) 2.37 (s, 3 H) 5.29 (s, 2 H) |
| | 1H-imidazol-5-yl)phenyl]- | | | 7.16 (d, 2 H) 7.26 (d, 1 H) 7.31-7.48 (m, |
| | 4-(pyridin-2- | | | 2H) 7.56 (t, 3 H) 7.86 (t, 1 H) 7.98 (d, 2 |
| | ylmethoxy)benzamide | | | H) 8.60 (d, 1 H) 9.77 (s, 1 H) 12.05 (br s, |
| | | | | 1 H) |
| 28 | N-{2-methyl-5-[5- | 452.43 | 453 | 2.27 (s, 3 H), 5.40 (s, 2 H), 7.20 (d, 2 H), |
| | (trifluoromethyl)-1H- | | | 7.39 (d, 1 H), 7.59 (t, 1 H), 7.76 (t, 2 H), |
| | imidazol-2-yl]phenyl}-4- | | | 7.89 (s, 1 H), 8.02 (m, 3 H), 8.11 (t, 1 H), |
| | (pyridin-2- | | | 8.72 (d, 1 H), 9.90 (s, 1 H), 13.23 (br s, 1 |
| | ylmethoxy)benzamide | | | H) |

### Example 29

### N-[5-(1H-imidazol-2-yl)-2-methyl-phenyl]-4-(pyridin-2-ylmethoxy)benzamide

Example 29 can be prepared in a similar fashion to Example 1 or by employing the method described below:

### 29a. 2-(4-methyl-3-nitrophenyl)-1H-imidazole

A 500 mL round bottom flask was charged with a magnetic stir bar and 4-methyl-3-nitrobenzaldehyde (5.0 g, 30.28 mmol). The vessel was cooled to 0° C. and EtOH (76 mL), NH₄OH (23.58 mL, 605.52 mmol), and oxalaldehyde (40% in water) (17.29 mL, 151.38 mmol) were added. The resulting mixture was then stirred at RT for 48 h before being concentrated in vacuo to afford the crude imidazole. The crude solid was washed with water (300 mL) and extracted with EtOAc (2×250 mL). The combined organic phases were dried with MgSO₄, filtered, and concentrated in vacuo to afford the crude product that was purified by ISCO MPLC (20% MeOH/DCM) to afford the title compound (2.81 g, 45.7%) as a brown solid. ¹H NMR (DMSO-d₆) δ 12.73 (br s, 1H) 8.48 (d, 1H) 8.11 (d, 1H) 7.53 (d, 1H) 7.27 (s, 1H) 7.02 (s, 1H) 2.45 (s, 3H). MS (M+H⁺)=204.

### 29b. 5-(1H-imidazol-2-yl)-2-methylaniline

A 250 mL round bottom flask was charged with a magnetic stir bar, 2-(4-methyl-3-nitrophenyl)-1H-imidazole (3.32 g, 16.34 mmol), and MeOH (163 mL). Palladium on activated carbon (500 mg, 10 wt %) was then added and the vessel was purged with hydrogen and placed under an atmosphere of hydrogen using a balloon. The mixture was allowed to stir for 24 h before being purged with nitrogen, filtered through a bed of Celite and concentrated in vacuo. The crude aniline was dissolved in MeOH (˜25 mL) and 10 mL of HCl (4N in dioxane) was added. Et₂O (˜200 mL) was added which caused the product to precipitate as the hydrochloride salt. The resulting solid was collected via vacuum filtration and dried in vacuo to afford the title compound as the hydrochloride salt (3.88 g, 96%). ¹H NMR (DMSO-d₆) δ 7.77-7.65 (m, 4H) 7.44 (d, 1H) 2.34 (s, 3H). MS (M+H⁺)=174.

### 29c. 4-(pyridin-2-ylmethoxy)benzoyl chloride

To 4-(pyridin-2-ylmethoxy)benzoic acid (2.181 g, 9.51 mmol) in THF (190 mL) and DMF (0.074 mL, 0.95 mmol) was added oxalyl chloride (8.33 mL, 95.14 mmol) dropwise at RT. The reaction mixture was heated to 50° C. for 4 h, then allowed to cool to RT. The mixture was concentrated, then triturated with Et₂O to yield the hydrochloride salt of the title compound (2.70 g, 100%). ¹H NMR (DMSO-d₆) δ 11.76 (br s, 1H), 8.75 (d, 1H), 8.21 (t, 1H), 7.91 (d, 2H), 7.81 (d, 1H), 7.68 (dd, 1H), 7.14 (d, 2H), 5.14 (s, 2H).

### 29d. N-(5-(1H-imidazol-2-yl)-2-methylphenyl)-4-(pyridin-2-ylmethoxy)benzamide

A 1 L round bottom flask was charged with a magnetic stir bar, 4-(pyridin-2-ylmethoxy)benzoyl chloride hydrochloride (11.86 g, 41.73 mmol), DCM (104 mL), pyridine (104 mL), and 5-(1H-imidazol-2-yl)-2-methylaniline hydrochloride (8.75 g, 41.73 mmol) was then added. The vessel was fitted with a reflux condenser and resulting reaction mixture was heated to 50° C. with stirring overnight. The vessel was allowed to cool to RT and the solvents were removed in vacuo. The crude residue was washed with 10% NaOH (˜250 mL) and extracted with EtOAc (2×500 mL). The combined organic extract was washed with brine, dried with MgSO₄, filtered, and concentrated in vacuo to afford the crude product which was pre-absorbed onto of silica gel (˜100 g) and purified via ISCO MPLC (10% MeOH/DCM) to afford the pure product as an off white solid. The obtained solid was recrystallized from 20 mL of MeOH, collected via vacuum filtration, and dried in vacuo to afford the title compound (10.45 g, 65.1%). ¹H NMR (DMSO-d₆) δ 12.44 (s, 1H) 9.84 (s, 1H) 8.59 (d, 1H) 7.98 (d, 2H) 7.90 (s, 1H) 7.85 (t, 1H) 7.72 (d, 1H) 7.54 (d, 1H) 7.39-7.31 (m, 2H) 7.16 (d, 2H) 7.11 (br s, 2H) 5.28 (s, 2H) 2.23 (s, 3H). MS (M+H⁺)=385.

### Example 30

### N-[5-(1H-benzoimidazol-2-yl)-2-methyl-phenyl]-4-(pyridin-2-ylmethoxy)benzamide

Example 30 can be prepared in a similar fashion to Example 1 or by utilizing the following method described below:

### 30a. 2-(4-methyl-3-nitrophenyl)-1H-benzo[d]imidazole

In a 200 mL round-bottomed flask was 4-methyl-3-nitrobenzoic acid (4 g, 22.08 mmol), benzene-1,2-diamine (2.388 g, 22.08 mmol), and DIPEA (7.71 mL, 44.16 mmol) in DMF (27.6 mL) to give a brown solution. The solution was cooled with an ice-water bath, and HATU (8.82 g, 23.19 mmol) was added. The reaction was stirred at RT for 2 h. The solution was added into 500 mL of water and stirred for 0.5 h. Filtration afforded a light yellow solid. The solid was placed in a 200 mL round-bottomed flask, and acetic acid (100 mL) was added to give a yellow suspension. The reaction was heated to 85° C. for 1 h. The reaction was concentrated under reduced pressure and diluted with sat. NaHCO₃ (100 mL). Filtration gave the title compound as a white solid. ¹H NMR (DMSO-d₆) [symbol] 2.60 (s, 3H), 7.24 (dd, 2H), 7.63 (dd, 2H), 7.70 (d, 1H), 8.40 (m, 1H), 8.78 (s, 1H).

### 30b. 5-(1H-benzo[d]imidazol-2-yl)-2-methylaniline

In a 500 mL round-bottomed flask was 2-(4-methyl-3-nitrophenyl)-1H-benzo[d]imidazole (5.0 g, 19.74 mmol) and iron(III) chloride (6.40 g, 1.97 mmol) in MeOH (200 mL) to give a yellow suspension. The mixture was heated to 75° C. for 20 min before hydrazine (21.25 mL, 236.91 mmol) was added. The reaction was kept stirring at that temp for 2 h. The solid residue was filtered off, and the filtrate was concentrated. To the residue was added water (50 mL) and DCM (100 mL). Partition, extraction with DCM (2×30 mL) and drying (Na₂SO₄) of the combined organic layers, followed by concentration gave the crude product. To the crude product was added DCM (100 mL) and it was stirred for 0.5 h, followed by filtration gave the title compound. ¹H NMR (DMSO-d₆) [symbol] 2.12 (s, 3H), 5.07 (s, 2H), 7.07 (d, 1H), 7.16 (m, 2H), 7.24 (d, 1H), 7.47 (m, 2H), 7.60 (dd, 1H), 12.64 (br s, 1H).

### 30c. N-[2-methyl-5-(2-methyl-1H-imidazol-4-yl)-phenyl]-4-(pyridin-2-ylmethoxy)benzamide

In a 200 mL round-bottomed flask was 4-(pyridin-2-ylmethoxy)benzoic acid (1.540 g, 6.72 mmol) in DCM to give a white suspension. SOCl₂ (0.981 mL, 13.44 mmol) was added. The reaction mixture was stirred at 40° C. for 3 h. The reaction was concentrated in vacuo to give 4-(pyridin-2-ylmethoxy)benzoyl chloride. To the residue was added pyridine (20 mL), and 5-(1H-benzo[d]imidazol-2-yl)-2-methylaniline (1.5 g, 6.72 mmol) was added. The reaction was heated to 60° C. for 1 h. Pyridine was removed under reduced pressure, and to the residue was added sat. NaHCO₃ (50 mL) and DCM (30 mL). The aqueous layer was extracted with DCM (2×15 mL), and concentration of the combined organic layers gave the crude product. The solid was dissolved in hot EtOH (20 mL), and after cooling down to 4° C., the precipitate was collected to give the title compound (0.894 g, 31% yield). ¹H NMR (DMSO-d₆) [symbol] 12.85 (s, 1H), 9.88 (s, 1H), 8.60 (d, 1H), 8.18 (d, 1H), 8.01 (d, 2H), 7.96 (dd, 1H), 7.85 (td, 1H), 7.60 (m, 3H), 7.49 (d, 1H), 7.44 (d, 1H), 7.36 (dd, 1H), 7.18 (m, 3H), 5.29 (s, 2H), 2.30 (s, 3H). MS (M+H⁺)=435.

### Example 31

### N-[4-(1H-benzoimidazol-2-yl)-2-methyl-phenyl]-4-(pyridin-2-ylmethoxy)benzamide

### 31a. N-(4-carbamoyl-2-methylphenyl)-4-(pyridin-2-ylmethoxy)benzamide

To 4-(pyridin-2-ylmethoxy)benzoic acid (2.05 g, 8.94 mmol), 4-amino-3-methylbenzamide (1.410 g, 9.39 mmol) and HATU (3.57 g, 9.39 mmol) in DMF (20 mL) was added DIPEA (4.69 mL, 26.83 mmol). The reaction mixture was heated to 50° C. for 10 h. After cooling to RT, the reaction mixture was poured into 1N NaOH. The precipitate was washed with water, followed by Et₂O, then dried under suction to yield the title compound (2.62 g, 81%). ¹H NMR (DMSO-d₆) δ 9.84 (s, 1H), 8.59 (d, 1H), 7.96 (d, 2H), 7.92 (s, 1H), 7.84 (m, 2H), 7.67 (dd, 1H), 7.53 (d, 1H), 7.36 (dd, 1H), 7.33 (d, 1H), 7.29 (s, 1H), 7.15 (d, 2H), 5.27 (s, 2H), 2.24 (s, 3H). MS (M+H⁺)=362.

### 31b. Methyl 3-methyl-4-(4-(pyridin-2-ylmethoxy)benzamido)benzoate

To N-(4-carbamoyl-2-methylphenyl)-4-(pyridin-2-ylmethoxy)benzamide (2.45 g, 6.78 mmol) in MeOH (67.8 mL) was added DMF-dimethylacetal (2.72 mL, 20.34 mmol). The reaction mixture was stirred at room temperature for 5 h, then at 45° C. for 24 h. The reaction was cooled to RT, then concentrated to 1.5 mL and poured into 10 mL of brine. The precipitate was filtered, washed with water, then Et₂O and dried under suction to yield the title compound (2.380 g, 93%). ¹H NMR (d₃-MeOD) δ 8.56 (d, 1H), 7.96 (d, 2H), 7.95 (m, 1H), 7.88 (m, 2H), 7.62 (d, 1H), 7.55 (d, 1H), 7.39 (dd, 1H), 7.15 (d, 2H), 5.28 (s, 2H), 3.89 (s, 3H), 2.35 (s, 3H). MS (M+H⁺)=377.

### 31c. 3-Methyl-4-(4-(pyridin-2-ylmethoxy)benzamido)benzoic acid

To methyl 3-methyl-4-(4-(pyridin-2-ylmethoxy)benzamido)benzoate (2.67 g, 7.09 mmol) in MeOH (79 mL) and water (19.70 mL) was added NaOH (0.426 g, 10.64 mmol). The reaction mixture was stirred at 50° C. for 2.5 h. After cooling to RT, 10.64 mL of 1M HCl and 100 mL of water were added and the mixture was cooled. The precipitate was collected by filtration, and washed with water then Et₂O to yield the title compound (2.56 g, 100%). ¹H NMR (DMSO-d₆) δ 12.81 (s, 1H), 9.80 (s, 1H), 8.59 (d, 1H), 7.96 (d, 2H), 7.85 (m, 2H), 7.77 (dd, 1H), 7.54 (t, 2H), 7.36 (dd, 1H), 7.16 (d, 2H), 5.28 (s, 2H), 2.29 (s, 3H). MS (M+H⁺)=363.

### 31d. N-(2-Aminophenyl)-3-methyl-4-(4-(pyridin-2-ylmethoxy)benzamido)benzamide

To 3-methyl-4-(4-(pyridin-2-ylmethoxy)benzamido)benzoic acid (0.145 g, 0.40 mmol), HATU (0.160 g, 0.42 mmol) and benzene-1,2-diamine (0.045 g, 0.41 mmol) was added DMF (1 mL) and DIPEA (0.210 mL, 1.20 mmol). The mixture was heated to 50° C. for 15 h. After cooling, 1 mL 1M NaOH and 9 mL brine were added, and the mixture was cooled. The precipitate was filtered, washed with water, Et₂O and dried under suction to yield the title compound (0.165 g, 91%). ¹H NMR (DMSO-d₆) δ 9.83 (s, 1H), 9.62 (s, 1H), 8.59 (d, 1H), 7.98 (d, 2H), 7.84 (m, 3H), 7.52 (t, 2H), 7.36 (dd, 1H), 716 (m, 3H), 6.96 (m, 1H), 6.77 (d, 1H), 6.59 (m, 1H), 5.28 (s, 2H), 4.89 (s, 2H), 2.31 (s, 3H). MS (M+H⁺)=453.

### 31e. N-(4-(1H-benzo[d]imidazol-2-yl)-2-methylphenyl)-4-(pyridin-2-ylmethoxy)benzamide

N-(2-aminophenyl)-3-methyl-4-(4-(pyridin-2-ylmethoxy)benzamido)benzamide (0.13 g, 0.29 mmol) in AcOH (2.87 mL) was heated to 80° C. for 1.5 h. After cooling to RT, the mixture was neutralized with saturated NaHCO₃ and the precipitate was filtered, washed with water, Et₂O and dried under suction to yield the title compound (0.077 g, 61.8%). ¹H NMR (DMSO-d₆) δ 12.84 (s, 1H), 9.81 (s, 1H), 8.59 (d, 1H), 8.08 (s, 1H), 7.98 (m, 3H), 7.85 (td, 1H), 7.64 (d, 1H), 7.53 (m, 3H), 7.36 (dd, 1H), 7.18 (m, 4H), 5.28 (s, 2H), 2.34 (s, 3H). MS (M+H⁺)=435.

### Example 32

### N-[5-(2,4-dimethyl1,3-thiazol-5-yl)-2-methyl-phenyl]-4-(pyridin-2-ylmethoxy)benzamide

### 32a. N-(5-iodo-2-methylphenyl)-4-(pyridin-2-ylmethoxy)benzamide

In a 10 mL round-bottomed flask was dissolved 5-iodo-2-methylaniline (5.171 g, 22.19 mmol), 4-(pyridin-2-ylmethoxy)benzoic acid, HCl (4.91 g, 18.49 mmol), DIPEA (9.66 mL, 55.47 mmol) and HATU (14.06 g, 36.98 mmol) in NMP (92 mL) to give an orange solution. The reaction was heated to 70° C. for 12 h, after which time, the reaction was poured into 1M aqueous NaOH (400 mL), and the resultant precipitate was removed via vacuum filtration. The filter cake was rinsed with water (200 mL), MTBE (100 mL), and dried under suction to yield the title compound (5.52 g, 67%) as a pale brown solid. ¹H NMR (DMSO-d₆) δ 2.19 (s, 3H) 5.29 (s, 2H) 7.08 (d, 1H) 7.16 (m, 2H) 7.29-7.43 (m, 1H) 7.50 (dd, 1H) 7.55 (d, 1H) 7.73 (d, 1H) 7.87 (td, 1H) 7.95 (m, 2H) 8.61 (d, 1H) 9.78 (s, 1H). MS (M+H⁺)=445.

### 32b. N-[5-(2,4-dimethyl1,3-thiazol-5-yl)-2-methyl-phenyl]-4-(pyridin-2-ylmethoxy)benzamide

A solution of N-(5-iodo-2-methylphenyl)-4-(pyridin-2-ylmethoxy)benzamide (0.45 mmol, 200 mg), 2,4-dimethyl-5-(4,4,5,5-tetramethyl-1,3,2-dioxaborolan-2-yl)thiazole (250 mg, 0.45 mmol), Cs₂CO₃ (1.8 mmol, 587 mg) and Pd(PPh₃)₄ (0.07 mmol 78 mg) in 1,4-dioxane (2 mL) and water (1 mL) was heated in microwave at 120° C. for 20 min. After the reaction mixture was cooled, water (2 mL) and EtOAc (4 mL) were added. The aqueous layer was removed using a pipette. The organic layer of the reaction was concentrated and the residue was purified using ISCO MPLC (5-10% MeOH/DCM) to afford product. The concentrated residue was acidified with HCl (2 mL, 4N in dioxane). Concentration of the acidic solution under vacuum provided the hydrochloride salt of the title compound (170 mg, 70%) as a yellow solid. ¹H NMR (DMSO-d₆) δ 2.26 (s, 3H) 2.41 (s, 3H) 2.66 (s, 3H) 5.43 (s, 2H) 7.13-7.28 (m, 3H) 7.36 (d, 1H) 7.45 (d, 1H) 7.59-7.71 (m, 1H) 7.79 (d, 1H) 8.00 (d, 2H) 8.08-8.29 (m, 1H) 8.75 (d, 1H) 9.87 (s, 1H). MS (M+H⁺)=430.

The following Examples 33-37 were prepared in a similar fashion to Example 32 utilizing commercially available starting materials:

| | | | MS | |
|---|---|---|---|---|
| Ex. | Name | MW | (M + H⁺) | ¹H NMR (δ ppm) |
| 33 | N-[2-methyl-5-(1- | 398.46 | 399 | (d₃-MeOD) 2.30 (s, 3 H) 3.93 (s, 3 H) |
| | methylpyrazol-4- | | | 5.45 (s, 2 H) 7.23 (m, 2 H) 7.28-7.36 (m, 1 H) |
| | yl)phenyl]-4-(pyridin-2- | | | 7.36-7.44 (m, 1 H) 7.53 (d, 1 H) 7.72 (d, |
| | ylmethoxy)benzamide | | | 1 H) 7.81 (s, 1 H) 7.86-7.99 (m, 2 H) |
| | | | | 8.03 (m, 2 H) 8.26 (td, 1 H) 8.73 (dd, 1 H) |
| 34 | N-[2-methyl-5-(2- | 398.46 | 399 | 2.29 (s, 3 H) 3.87 (s, 3 H) 5.44 (s, 2 H) |
| | methylpyrazol-3- | | | 6.39 (d, 1 H) 7.21 (m, 2 H) 7.28-7.44 (m, |
| | yl)phenyl]-4-(pyridin-2- | | | 2 H) 7.49 (dd, 2 H) 7.67 (d, 1 H) 7.81 (d, 1 |
| | ylmethoxy)benzamide | | | H) 8.01 (m, 2 H) 8.19 (td, 1 H) 8.76 (d, 1 |
| | | | | H) 9.90 (s, 1 H) |
| 35 | N-[2-methyl-5-(1,3,5- | 426.52 | 427 | 2.16 (s, 3 H) 2.24 (s, 6 H) 3.72 (s, 3 H) |
| | trimethylpyrazol-4- | | | 5.40 (s, 3 H) 7.05 (dd, 1 H) 7.13-7.25 (m, |
| | yl)phenyl]-4-(pyridin-2- | | | 2 H) 7.30 (d, 1 H) 7.60 (d, 1 H) 7.74 (d, 1 |
| | ylmethoxy)benzamide | | | H) 7.99 (d, 2 H) 8.11 (d, 1 H) 8.72 (d, 1 H) |
| | | | | 9.79 (s, 1 H) |
| 36 | N-[2-methyl-5-[1-(2- | 440.54 | 441 | 0.86 (d, 6 H) 2.04-2.18 (m, 1 H) 2.20 (s, |
| | methylpropyl)pyrazol- | | | 3 H) 3.92 (d, 2 H) 5.43 (s, 2 H) |
| | 4-yl]phenyl]-4-(pyridin- | | | 7.14-7.28 (m, 3 H) 7.38 (dd, 1 H) 7.50 (d, 1 H) |
| | 2-ylmethoxy)benzamide | | | 7.66 (d, 1 H) 7.77-7.90 (m, 2 H) 8.01 (d, 2 H) |
| | | | | 8.13-8.27 (m, 2 H) 8.76 (d, 1 H) 9.82 (s, |
| | | | | 1 H) |
| 37 | N-[2-methyl-5-[5- | 452.43 | 453 | 2.26 (s, 3 H) 5.29 (s, 2 H) 7.06-7.27 (m, 3 |
| | (trifluoromethyl)-2H- | | | H) 7.29-7.41 (m, 2 H) 7.55 (d, 1 H) |
| | pyrazol-3-yl]phenyl]-4- | | | 7.60-7.69 (m, 1 H) 7.78-7.92 (m, 2 H) 8.00 (d, |
| | (pyridin-2- | | | 2 H) 8.52-8.67 (m, 1 H) 9.89 (s, 1 H) |
| | ylmethoxy)benzamide | | | 14.03 (d, 1 H) |

### Example 38

### N-(2-methyl-5-(5-methyl-1,3,4-oxadiazol-2-yl)phenyl)-4-(pyridin-2-ylmethoxy)benzamide

### 38a. N-(5-(2-acetylhydrazinecarbonyl)-2-methylphenyl)-4-(pyridin-2-ylmethoxy)benzamide

To a solution of 4-methyl-3-(4-(pyridin-2-ylmethoxy)benzamido)benzoic acid (560 mg, 1.55 mmol), acetohydrazide (229 mg, 3.09 mmol) and DIPEA (1080 μl, 6.18 mmol) in DMF (5.15 mL) was added HATU (1175 mg, 3.09 mmol). The reaction was stirred overnight. The reaction was concentrated under vacuum and the residue was purified using ISCO MPLC (0-10% MeOH/DCM) to afford the title compound (647 mg, 99%). MS (M+H⁺)=419.

### 38b. N-(2-methyl-5-(5-methyl-1,3,4-oxadiazol-2-yl)phenyl)-4-(pyridin-2-ylmethoxy)benzamide

To a solution of N-(5-(2-acetylhydrazinecarbonyl)-2-methylphenyl)-4-(pyridin-2-ylmethoxy)benzamide (550 mg, 1.31 mmol), PPh₃ (1034 mg, 3.94 mmol) and DIPEA (918 μl, 5.26 mmol) in MeCN (11 mL) was added perchloroethane (622 mg, 2.63 mmol). The reaction was stirred overnight. The reaction was concentrated and the residue was purified using ISCO MPLC (EtOAc to 10% MeOH/DCM) to afford the title compound (180 mg, 34%). ¹H NMR (DMSO-d₆) δ 2.33 (s, 3H) 2.58 (s, 3H) 5.30 (s, 2H) 7.18 (d, 2H) 7.37 (ddd, 4.85, 1H) 7.52 (dd, 2H) 7.75 (dd, 1H) 7.86 (td, 1H) 7.91-8.20 (m, 3H) 8.51-8.73 (m, 1H) 9.90 (s, 1H). MS (M+H⁺)=401.

### Example 39

### N-(5-(4,5-dimethyl-4H-1,2,4-triazol-3-yl)-2-methylphenyl)-4-(pyridin-2-ylmethoxy)benzamide

### 39a. Methyl 4-methyl-3-(4-(pyridin-2-ylmethoxy)benzamido)benzoate

To a solution of 4-(pyridin-2-ylmethoxy)benzoic acid (1.0 g, 4.36 mmol), methyl 3-amino-4-methylbenzoate (0.735 g, 4.45 mmol) and HATU (1.742 g, 4.58 mmol) in DMF was added DIPEA (2.286 mL, 13.09 mmol). The reaction was stirred at 50° C. for 16 h. After cooling to RT, the solution was poured into 1M NaOH (100 mL). The precipitate was filtered, washed with water, followed by Et₂O to yield a white solid. The solid was dried under suction to yield the title compound (38.2%) as the monohydrate.

### 39b. 4-methyl-3-(4-(pyridin-2-ylmethoxy)benzamido)benzoic acid

Methyl 4-methyl-3-(4-(pyridin-2-ylmethoxy)benzamido)benzoate (627 mg, 1.67 mmol) and NaOH (133 mg, 3.33 mmol) were dissolved in MeOH (12.5 mL) and water (4.17 mL), stirred at RT for 20 h, then at 50° C. for 1.5 h. After cooling to RT, 1M HCl (3.3 mL) was added and the precipitate was filtered, washed with water, followed by Et₂O, to yield the title compound (99%) as a white solid.

### 39c. N-(5-(4,5-dimethyl-4H-1,2,4-triazol-3-yl)-2-methylphenyl)-4-(pyridin-2-ylmethoxy)benzamide

A solution of N-(2-methyl-5-(5-methyl-1,3,4-oxadiazol-2-yl)phenyl)-4-(pyridin-2-ylmethoxy)benzamide (100 mg, 0.25 mmol) in MeNH₂/EtOH (30% wt.) was heated in a microwave for 7 h at 130° C. The reaction was concentrated in vacuo and then purified using ISCO MPLC (0-40% MeOH/DCM) to afford the title compound (27 mg, 26%). ¹H NMR (DMSO-d₆) δ 2.31 (s, 3H) 2.40 (s, 3H) 3.59 (s, 3H) 5.29 (s, 2H) 7.17 (m, 2H) 7.37 (ddd, 4.85, 1H) 7.42-7.48 (m, 2H) 7.54 (d, 1H) 7.65 (s, 1H) 7.86 (td, 1H) 7.98 (m, 2H) 8.60 (d, 1H) 9.88 (s, 1H). MS (M+H⁺)=414.

### Example 40

### N-(2-methyl-5-(5-methyl-1,2,4-oxadiazol-3-yl)phenyl)-4-(pyridin-2-ylmethoxy)benzamide

### 40a. N-(5-cyano-2-methylphenyl)-4-(pyridin-2-ylmethoxy)benzamide

A solution of 4-(pyridin-2-ylmethoxy)benzoyl chloride hydrochloride (644 mg, 2.60 mmol) and 3-amino-4-methylbenzonitrile (515 mg, 3.90 mmol) in pyridine was stirred for 16 h. Concentration of the reaction mixture under reduced pressure afforded a crude residue, which was purified using ISCO MPLC (40-100% EtOAc/hexane) to yield the title compound. MS (M+H⁺)=344.

### 40b. N-(5-(N-hydroxycarbamimidoyl)-2-methylphenyl)-4-(pyridin-2-ylmethoxy)benzamide

A suspension of N-(5-cyano-2-methylphenyl)-4-(pyridin-2-ylmethoxy)benzamide (170 mg, 0.5 mmol), hydroxylamine hydrochloride (37.8 mg, 0.54 mmol) and NaHCO₃ (40.3 mg, 0.43 mmol) in MeOH (0.5 mL) was heated in a microwave at 70° C. for 1 h. A precipitate formed, which was collected and washed with MeOH and water to yield the title compound. MS (M+H⁺)=377.

### 40c. N-(2-methyl-5-(5-methyl-1,2,4-oxadiazol-3-yl)phenyl)-4-(pyridin-2-ylmethoxy)benzamide

A solution of N-(5-(N-hydroxycarbamimidoyl)-2-methylphenyl)-4-(pyridin-2-ylmethoxy)benzamide (160 mg, 0.43 mmol) and acetic anhydride (347 mg, 3.47 mmol) in 1,4-dioxane (1.4 mL) was heated in a microwave at 150° C. for 1 h. The reaction was concentrated in vacuo and the residue was purified by Gilson HPLC (10-70% MeCN/0.1% TFA in water) to afford the title compound (70 mg, 41%). ¹H NMR (d₃-MeOD) δ 2.38 (s, 4H) 2.66 (s, 3H) 5.31 (s, 2H) 7.11-7.26 (m, 2H) 7.37-7.50 (m, 2H) 7.66 (d, 1H) 7.83-7.95 (m, 2H) 7.97-8.09 (m, 3H) 8.52-8.62 (m, 1H). MS (M+H⁺)=401.

### Example 41

### N-(5-(1H-imidazol-2-yl)-2-methylphenyl)-4-(3-methoxybenzyloxy)benzamide 41a. Methyl 4-(3-methoxybenzyloxy)benzoate

In a 250 mL round-bottomed flask was added methyl 4-hydroxybenzoate (2.0 g, 13.15 mmol), 1-(bromomethyl)-3-methoxybenzene (2.64 g, 13.15 mmol), and K₂CO₃ (4.54 g, 32.86 mmol) in MeCN (50 mL) to give a white suspension. The reaction was stirred overnight at RT filtered, and concentrated in vacuo to give the title compound as a white solid (3.5 g, 98% yield). ¹H NMR (DMSO-d₆) [symbol] 3.76 (s, 3H), 3.81 (s, 3H), 5.17 (s, 2H), 6.91 (m, 1H), 7.02 (m, 2H), 7.12 (d, 2H), 7.31 (t, 1H), 7.92 (d, 2H).

### 41b. 4-(3-Methoxybenzyloxy)benzoic acid

In a 500 mL round-bottomed flask was added methyl 4-(3-methoxybenzyloxy)benzoate (3.50 g, 12.85 mmol) and LiOH (1.539 g, 64.27 mmol) in EtOH (200 mL) to give a colorless suspension. The reaction was heated to 60° C. for 2 h. After cooling to RT and concentration under reduced pressure, water (100 mL) was added. Using aq. HCl (6 N) the pH was adjusted to 2, and at that time a precipitate was observed. The precipitate was collected by filtration to give the title compound. ¹H NMR (DMSO-d₆) [symbol] 3.76 (s, 3H), 5.16 (s, 2H), 6.90 (m, 1H), 7.03 (m, 2H), 7.09 (d, 2H), 7.31 (t, 1H), 7.89 (d, 2H), 12.64 (s, 1H).

### 41c. 3-(4-(3-Methoxybenzyloxy)benzamido)-4-methylphenylboronic acid

In a 100 mL round-bottomed flask was dissolved 4-(3-methoxybenzyloxy)benzoic acid (0.47 g, 1.82 mmol), 3-amino-4-methylphenylboronic acid (0.288 g, 1.91 mmol), and DIPEA (0.795 mL, 4.55 mmol) in DMF (2 mL) to give a brown solution. The reaction mixture was cooled to 0° C. before HATU (0.727 g, 1.91 mmol) was added. After the reaction mixture was warmed to RT, it was stirred for additional 3 h. Water (50 mL) was added and filtration afforded the title compound as a brown solid. MS (M+H⁺)=392.

### 41d. N-(5-(1H-imidazol-2-yl)-2-methylphenyl)-4-(3-methoxybenzyloxy)benzamide

In a 10 mL vial was added 2-bromo-1H-imidazole (0.085 g, 0.58 mmol), 3-(4-(3-methoxybenzyloxy)benzamido)-4-methylphenylboronic acid (0.15 g, 0.38 mmol), and K₂CO₃ (0.094 g, 0.96 mmol) in dioxane (4 mL) to give a colorless suspension. The reaction mixture was diluted with water (1 mL). Nitrogen was bubbled in for 20 min before Pd(PPh₃)₄ (0.044 g, 0.04 mmol) was added. The reaction was heated to 100° C. for 2.5 h. After concentration under reduced pressure the crude product was purified by Gilson HPLC (MeCN/10 mM NH₄OAc in water) to give the title compound (0.024 g, 16% yield). ¹H NMR (DMSO-d₆) δ 2.24 (s, 3H), 3.77 (s, 3H), 5.19 (s, 2H), 6.92 (s, 1H), 7.05 (br s, 2H), 7.15 (d, 3H), 7.32 (m, 2H), 7.59 (br s, 1H), 7.72 (m, 1H), 7.91 (d, 1H), 7.98 (d, 2H), 9.84 (s, 1H), 12.59 (br s, 1H). MS (M+H⁺)=414.

The following Examples 42-57 were prepared in a similar fashion to Example 41 utilizing commercially available starting materials:

| | | | MS | |
|---|---|---|---|---|
| Ex. | Name | MW | (M + H⁺) | ¹H NMR (δ ppm) |
| 42 | 4-[(3,5- | 471.55 | 472 | 2.10 (s, 3 H), 2.27 (s, 3 H), 3.69 (s, 3 |
| | dimethoxyphenyl)methoxy]- | | | H), 3.75 (s, 6 H), 5.15 (s, 2 H), |
| | N-[5-(1,4-dimethylimidazol- | | | 6.46 (s, 1 H), 6.63 (d, 2 H), 6.92 (s, 1 H), |
| | 2-yl)-2-methyl- | | | 7.13 (d, 2 H), 7.34 (m, 1 H), 7.44 (s, 1 |
| | phenyl]benzamide | | | H), 7.65 (s, 1 H), 7.97 (d, 2 H), |
| | | | | 9.80 (s, 1 H) |
| 43 | 4-[(3,5- | 457.53 | 458 | 2.34 (s, 6 H), 3.75 (s, 6 H), 5.16 (s, 2 |
| | dimethoxyphenyl)methoxy]- | | | H), 6.47 (m, 1 H), 6.63 (d, 2 H), |
| | N-[2-methyl-5-(4-methyl-1H- | | | 7.16 (d, 2 H), 7.50 (s, 1 H), 7.56 (d, 1 H), |
| | imidazol-2- | | | 7.83 (m, 1 H), 8.02 (m, 3 H), 9.95 (s, |
| | yl)phenyl]benzamide | | | 1 H), 14.50 (s, 2 H) |
| 44 | 4-[(2-cyanophenyl)methoxy]- | 422.49 | 423 | 2.35 (s, 6 H), 5.36 (s, 2 H), 7.22 (d, 2 |
| | N-[2-methyl-5-(4-methyl-1H- | | | H), 7.49 (s, 1 H), 7.60 (m, 2 H), |
| | imidazol-2- | | | 7.78 (d, 2 H), 7.88 (dd, 1 H), 7.95 (d, 1 H), |
| | yl)phenyl]benzamide | | | 8.06 (m, 3 H), 10.02 (s, 1 H), 14.58 (s, |
| | | | | 1 H) |
| 45 | 4-[(2-cyanophenyl)methoxy]- | 436.51 | 437 | 2.32 (s, 3 H), 2.38 (s, 3 H), 3.84 (s, 3 |
| | N-[5-(1,4-dimethylimidazol- | | | H), 5.36 (s, 2 H), 7.22 (d, 2 H), |
| | 2-yl)-2-methyl- | | | 7.53 (s, 1 H), 7.61 (dt, 3 H), 7.78 (d, 2 H), |
| | phenyl]benzamide | | | 7.85 (s, 1 H), 7.95 (d, 1 H), 8.03 (d, 2 |
| | | | | H), 10.02 (s, 1 H), 14.56 (s, 1 H) |
| 46 | 4-[(2-cyanophenyl)methoxy]- | 408.46 | 409 | 2.35 (s, 3 H), 5.36 (s, 2 H), 7.22 (d, 2 |
| | N-[5-(1H-imidazol-2-yl)-2- | | | H), 7.60 (m, 2 H), 7.78 (m, 4 H), |
| | methyl-phenyl]benzamide | | | 7.85 (m, 1 H), 7.95 (d, 1 H), 8.03 (d, 2 H), |
| | | | | 8.10 (s, 1 H), 9.99 (s, 1 H), 14.64 (s, 1 |
| | | | | H) |
| 47 | 4-[(3,5- | 443.5 | 444 | 2.34 (s, 3 H), 3.75 (s, 6 H), 5.16 (s, 2 |
| | dimethoxyphenyl)methoxy]- | | | H), 6.47 (s, 1 H), 6.63 (d, 2 H), |
| | N-[5-(1H-imidazol-2-yl)-2- | | | 7.16 (d, 2 H), 7.56 (d, 1 H), 7.79 (s, 2 H), |
| | methyl-phenyl]benzamide | | | 7.86 (dd, 1 H), 8.00 (d, 2 H), 8.10 (s, 1 |
| | | | | H), 9.96 (s, 1 H), 14.70 (s, 1 H) |
| 48 | 4-[(3,5- | 471.55 | 472 | 2.36 (s, 3 H), 2.38 (s, 3 H), 3.74 (s, 3 |
| | dimethoxyphenyl)methoxy]- | | | H), 5.36 (s, 2 H), 7.22 (d, 2 H), |
| | N-[5-(1,5-dimethylimidazol- | | | 7.52 (m, 1 H), 7.60 (m, 3 H), 7.79 (m, 3 H), |
| | 2-yl)-2-methyl- | | | 7.95 (d, 1 H), 8.02 (d, 2 H), 10.00 (s, 1 |
| | phenyl]benzamide | | | H), 14.48 (s, 1 H) |
| 49 | 4-[(2-cyanophenyl)methoxy]- | 436.51 | 437 | 2.34 (s, 3 H), 3.75 (s, 6 H), 5.16 (s, 2 |
| | N-[5-(1,5-dimethylimidazol- | | | H), 6.47 (s, 1 H), 6.63 (d, 2 H), |
| | 2-yl)-2-methyl- | | | 7.16 (d, 2 H), 7.56 (d, 1 H), 7.79 (s, 2 H), |
| | phenyl]benzamide | | | 7.86 (dd, 1 H), 8.00 (d, 2 H), 8.10 (s, 1 |
| | | | | H), 9.96 (s, 1 H), 14.70 (s, 1 H) |
| 50 | 4-[(3,5- | 457.53 | 458 | 2.37 (s, 3 H), 3.75 (s, 6 H), 3.89 (s, 3 |
| | dimethoxyphenyl)methoxy]- | | | H), 5.16 (s, 2 H), 6.46 (s, 1 H), |
| | N-[2-methyl-5-(1- | | | 6.63 (d, 2 H), 7.15 (d, 2 H), 7.58 (s, 2 H), |
| | methylimidazol-2- | | | 7.81 (s, 1 H), 7.85 (m, 2 H), 7.99 (d, 2 |
| | yl)phenyl]benzamide | | | H), 9.97 (s, 1 H) |
| 51 | 4-[(2-cyanophenyl)methoxy]- | 422.49 | 423 | 2.38 (s, 3 H), 3.89 (s, 3 H), 5.36 (s, 2 |
| | N-[2-methyl-5-(1- | | | H), 7.22 (d, 2 H), 7.61 (m, 3 H), |
| | methylimidazol-2- | | | 7.79 (m, 3 H), 7.85 (m, 2 H), 7.95 (d, 1 H), |
| | yl)phenyl]benzamide | | | 8.02 (d, 2 H), 10.00 (s, 1 H) |
| 52 | 4-[(2-cyanophenyl)methoxy]- | 422.49 | 422 | 2.33 (s, 3 H), 3.85 (s, 3 H), 5.36 (s, 2 |
| | N-[2-methyl-5-(3- | | | H), 7.21 (d, 2 H), 7.40 (m, 1 H), |
| | methylimidazol-4- | | | 7.47 (m, 1 H), 7.61 (m, 2 H), 7.78 (m, 2 H), |
| | yl)phenyl]benzamide | | | 7.88 (d, 1 H), 7.95 (d, 1 H), 8.01 (d, 2 |
| | | | | H), 9.19 (s, 1 H), 9.92 (s, 1 H) |
| 53 | 4-[(2-cyanophenyl)methoxy]- | 422.49 | 423 | 2.29 (s, 3 H), 3.88 (s, 3 H), 5.36 (s, 2 |
| | N-[2-methyl-5-(1- | | | H), 7.21 (d, 2 H), 7.43 (d, 1 H), |
| | methylimidazol-4- | | | 7.61 (m, 2 H), 7.78 (d, 2 H), 7.81 (s, 1 H), |
| | yl)phenyl]benzamide | | | 7.95 (d, 1 H), 8.03 (d, 2 H), 8.14 (s, 1 |
| | | | | H), 9.11 (s, 1 H), 9.92 (s, 1 H) |
| 54 | 4-[(2-cyanophenyl)methoxy]- | 422.49 | 423 | 2.31 (s, 3 H), 2.45 (s, 3 H), 5.36 (s, 2 |
| | N-[2-methyl-5-(5-methyl-1H- | | | H), 7.21 (s, 2 H), 7.44 (m, 2 H), |
| | imidazol-4- | | | 7.61 (ddd, 4.33, 1 H), 7.66 (s, 1 H), 7.78 (d, |
| | yl)phenyl]benzamide | | | 2 H), 7.95 (d, 1 H), 8.02 (d, 2 H), |
| | | | | 9.11 (s, 1 H), 9.91 (s, 1 H), 14.55 (s, 1 H) |
| 55 | 4-[(2-cyanophenyl)methoxy]- | 436.51 | 436 | 2.33 (s, 3 H), 2.64 (s, 3 H), 3.68 (s, 3 |
| | N-[5-(2,3-dimethylimidazol- | | | H), 5.36 (s, 2 H), 7.21 (d, 2 H), |
| | 4-yl)-2-methyl- | | | 7.33 (d, 1 H), 7.47 (d, 1 H), 7.61 (m, 2 H), |
| | phenyl]benzamide | | | 7.74 (s, 1 H), 7.78 (m, 2 H), 7.95 (d, 1 |
| | | | | H), 8.01 (d, 2 H), 9.91 (s, 1 H) |
| 56 | 4-[(3,4-dimethoxypyridin-2- | 444.49 | 445 | 2.25 (s, 3 H), 3.81 (s, 3 H), 3.92 (s, 3 |
| | yl)methoxy]-N-[5-(1H- | | | H), 5.20 (s, 2 H), 7.16 (m, 5 H), |
| | imidazol-2-yl)-2- | | | 7.33 (d, 1 H), 7.74 (d, 1 H), 7.92 (s, 1 H), |
| | methylphenyl]benzamide | | | 7.99 (d, 2 H), 8.24 (d, 1 H), 9.84 (s, 1 |
| | | | | H), 12.47 (br s, 1 H) |
| 57 | 4-[(6-chloro-1,3-benzodioxol- | 461.90 | 462 | 2.33 (s, 3 H), 5.14 (s, 2 H), 6.11 (s, 1 |
| | 5-yl)methoxy]-N-[5-(1H- | | | H), 7.19 (m, 6 H), 7.52 (d, 1 H), |
| | imidazol-2-yl)-2- | | | 7.65 (s, 2 H), 7.85 (d, 1 H), 8.00 (s, 1 H), |
| | methylphenyl]benzamide | | | 8.05 (d, 2 H), 9.96 (s, 1 H) |

### Example 58

### N-(5-(1H-imidazol-2-yl)-2-methylphenyl)-4-(benzyloxy)benzamide

Example 58 can be prepared in a similar fashion to Example 41 or by employing the following method described below:

### 58a. 4-(benzyloxy)-N-(5-(hydroxymethyl)-2-methylphenyl)benzamide

To a mixture of 4-(benzyloxy)benzoic acid (26 g, 113.91 mmol) and (3-amino-4-methylphenyl)methanol (15.63 g, 113.91 mmol), HATU (52.0 g, 136.70 mmol) in DMF (250 mL) was added DIPEA (39.8 mL, 227.83 mmol) at 0° C. The mixture was stirred at 80° C. overnight. Water (˜1 L) was added to the mixture, and the solid precipitate was collected by filtration, washed with water, dried, then washed with Et₂O (2×), and dried to yield the title compound as a light yellow solid (38.0 g, 96%). MS (M+H⁺)=348.

### 58b. 4-(benzyloxy)-N-(5-formyl-2-methylphenyl)benzamide

A solution of oxalyl dichloride (21.52 g, 169.54 mmol) in 200 mL of anhydrous DCM was cooled to −78° C., then DMSO (26.5 g, 339.08 mmol) was added to the mixture dropwise and the mixture was stirred for 15 min before a suspension of 4-(benzyloxy)-N-(5-(hydroxymethyl)-2-methylphenyl)benzamide (38 g, 109.38 mmol) in 300 mL of DCM was added over 40 min. After 1 h at −78° C., TEA (73.2 mL, 525.03 mmol) was added and the reaction mixture was allowed to warmed to RT for 1.5 h. 400 mL of sat. NaHCO₃ was added to the mixture, and the aqueous layer was extracted with DCM. The combined organic layers were dried over Na₂SO₄, filtered, washed with DCM, and concentrated in vacuo. The residue was purified by ISCO MPLC (40-55% EtOAc/hexane) to yield the title compound as a white solid (15.90 g, 42.1%). MS (M+H⁺)=346.

### 58c. N-(5-(1H-imidazol-2-yl)-2-methylphenyl)-4-(benzyloxy)benzamide

To a suspension of 4-(benzyloxy)-N-(5-formyl-2-methylphenyl)benzamide (15.9 g, 46.03 mmol) in MeOH (400 mL) was added NH₄OH (27.2 mL, 230.17 mmol) at 0° C. followed by the addition of oxalaldehyde (52.8 mL, 460.35 mmol). The reaction mixture was then stirred at RT for 1 day. Another portion of NH₄OH (27.2 mL, 230.17 mmol) and oxalaldehyde (52.8 mL, 460.35 mmol) were added to the reaction mixture and stirred at RT for 1 day. One final portion of NH₄OH (27.2 mL, 230.17 mmol) and oxalaldehyde (52.8 mL, 460.35 mmol) were added to the reaction mixture and stirred at RT for 2 days. Water (˜1.5 L) was added to the mixture, and the solid was collected by filtration, washed with water, dried, washed with Et₂O, and then dried to afford a grey solid as the crude product (17.60 g), which was purified by ISCO MPLC (EtOAc/hexane) to yield the title compound as a white solid (12 g, 68%). ¹H NMR (DMSO-d₆) δ 2.24 (s, 3H), 5.22 (s, 2H), 7.01 (br s, 1H), 7.15 (m, 3H), 7.37 (m, 4H), 7.49 (m, 2H), 7.73 (dd, 1H), 7.91 (s, 1H), 7.99 (d, 2H), 9.85 (s, 1H), 12.49 (br s, 1H). MS (M+H⁺)=384.

### Example 59

### 4-[(4-Fluorophenyl)methoxy]-N-[5-(1H-imidazol-2-yl)-2-methylphenyl]benzamide

### 59a. N-(5-(1H-imidazol-2-yl)-2-methylphenyl)-4-hydroxybenzamide

In a 500 mL pressure vessel was added N-(5-(1H-imidazol-2-yl)-2-methylphenyl)-4-(benzyloxy)benzamide (2.77 g, 7.22 mmol) in MeOH (100 mL) to give a white suspension. Nitrogen gas was bubbled in for 20 min before Pd/C (0.384 g, 10%) was added. The vessel was purged with hydrogen three times, and then stirred under hydrogen at 55 psi for 12 h. After purging the vessel with nitrogen, the suspension was filtered, and the filter cake was washed with MeOH (3×15 mL) and EtOAc (2×15 mL). The combined organic filtrates were concentrated to give the title compound (1.8 g, 85% yield). ¹H NMR (DMSO-d₆) δ 2.23 (s, 3H), 6.87 (d, 2H), 7.00 (br s, 1H), 7.20 (br s, 1H), 7.32 (d, 1H), 7.71 (dd, 1H), 7.88 (m, 3H), 9.72 (s, 1H), 10.15 (br s, 1H), 12.44 (br s, 1H).

### 59b. 4-[(4-Fluorophenyl)methoxy]N-[5-(1H-imidazol-2-yl)-2-methylphenyl]benzamide

In a 10 mL vial was added N-(5-(1H-imidazol-2-yl)-2-methylphenyl)-4-hydroxybenzamide (0.09 g, 0.31 mmol), K₂CO₃ (0.17 g, 1.23 mmol), and 4-fluorobenzyl chloride (0.074 g, 0.34 mmol) in NMP (1.0 mL) to give a colorless suspension. The reaction was heated to 160° C. for 30 min. After cooling down to RT, the solution was purified by Gilson HPLC (MeCN/0.1% TFA in water) to give a solid residue. To the residue was added MeOH (0.5 mL) and HCl in Et₂O (1.5 mL, 2 M). Concentration gave the title compound as the HCl salt (0.033 g, 27% yield). ¹H NMR (DMSO-d₆) δ 2.34 (s, 3H), 5.20 (s, 2H), 7.17 (m, 2H), 7.25 (t, 2H), 7.56 (m, 3H), 7.78 (s, 2H), 7.89 (dd, 1H), 8.01 (m, 2H), 8.11 (s, 1H), 9.98 (s, 1H), 14.74 (br s, 1H). MS (M+H⁺)=402.

The following Examples 60-64 were prepared in a similar fashion to Example 59 utilizing commercially available starting materials:

| | | | MS | |
|---|---|---|---|---|
| Ex. | Name | MW | (M + H⁺) | ¹H NMR (δ ppm) |
| 60 | N-[5-(1H-imidazol-2-yl)-2- | 461.54 | 462 | 2.34 (s, 3 H), 3.24 (s, 3 H), 5.37 (s, 2 H), |
| | methylphenyl]-4-{[4- | | | 7.19 (d, 2 H), 7.56 (d, 1 H), 7.76 (m, 4 H), |
| | (methylsulfonyl)benzyl]oxy}benzamide | | | 7.86 (dd, 1 H), 8.00 (dd, 4 H), 8.10 (s, 1 |
| | | | | H), 9.98 (s, 1 H), 14.68 (br s, 1 H) |
| 61 | 4-[(3-cyanobenzyl)oxy]-N- | 408.46 | 409 | 2.34 (s, 3 H), 5.29 (s, 2 H), 7.20 (m, 2 H), |
| | [5-(1H-imidazol-2-yl)-2- | | | 7.56 (d, 1 H), 7.65 (t, 1 H), 7.85 (m, 5 H), |
| | methylphenyl]benzamide | | | 7.97 (s, 1 H), 8.02 (m, 2 H), 8.10 (s, 1 H), |
| | | | | 9.99 (s, 1 H), 14.72 (br s, 1 H) |
| 62 | 4-{[4- | 413.48 | 414 | 2.34 (s, 3 H), 4.51 (s, 2 H), 5.20 (s, 2 H), |
| | (hydroxymethyl)benzyl]oxy}- | | | 7.16 (m, 2 H), 7.35 (m, 2 H), 7.43 (m, 2 |
| | N-[5-(1H-imidazol-2-yl)-2- | | | H), 7.56 (d, 1 H), 7.78 (s, 2 H), 7.86 (dd, 1 |
| | methylphenyl]benzamide | | | H), 8.00 (m, 2 H), 8.10 (d, 1 H), 9.96 (s, 1 |
| | | | | H), 14.70 (br s, 1 H) |
| 63 | N-[5-(1H-imidazol-2-yl)-2- | 413.48 | 414 | 2.34 (s, 3 H), 3.84 (s, 3 H), 5.16 (s, 2 H), |
| | methylphenyl]-4-[(2- | | | 6.98 (t, 1 H), 7.08 (d, 1 H), 7.16 (m, 2 H), |
| | methoxybenzyl)oxy]benzamide | | | 7.37 (td, 1 H), 7.42 (d, 1 H), 7.56 (d, 1 H), |
| | | | | 7.80 (s, 2 H), 7.89 (m, 1 H), 8.01 (m, 2 H), |
| | | | | 8.11 (s, 1 H), 9.99 (s, 1 H), 14.77 (br s, 1 |
| | | | | H) |
| 64 | N-[5-(1H-imidazol-2-yl)-2- | 451.45 | 452 | 2.27 (s, 3 H), 5.21 (s, 2 H), 6.82 (d, 2 H), |
| | methylphenyl]-4-{[2- | | | 6.86 (br s, 1 H), 7.19 (br s, 1 H), 7.38 (t, |
| | (trifluoromethyl)benzyl]oxy}benzamide | | | 1 H), 7.48 (t, 1 H), 7.64 (m, 4 H), 7.86 (br |
| | | | | s, 3 H), 9.18 (br s, 1 H), 14.25 (br s, 1 H) |

### Example 65

### N-(2-methyl-5-phenyl-phenyl)-4-(pyridin-2-ylmethoxy)benzamide

To a vial containing 4-methylbiphenyl-3-amine (0.70 mmol), dioxane (3 mL), and DIPEA (0.30 mL, 1.68 mmol), a 1.83 M solution of the 4-(pyridin-2-ylmethoxy)benzoyl chloride hydrochloride (0.56 mmol) in DMF was added. The reaction was stirred for 12 h at RT. The reaction was heated to 60° C. for 4 h, then poured into 1M NaOH (5 mL), and added DCM (2 mL). The phases were separated using SPE phase separation cartridges, and the organic layer was diluted to a total volume of 5 mL DCM and incubated with 3 eq. of MP-isocyanate resin for 18 h. The resin was removed via filtration, and rinsed with 2 mL DCM. The organic layers were combined and concentrated to dryness. The resulting oil was purified via Gilson HPLC (MeCN/10 mM NH₄OAc in water) to give the title compound (5 mg, 2%). ¹H NMR (CDCl₃) δ 8.65 (d, 1H) 8.26 (d, 1H) 7.90 (m, 2H) 7.80 (td, 1H) 7.55-7.71 (m, 4H) 7.41-7.45 (m, 2H) 7.28-7.40 (m, 4H) 7.12 (m, 2H) 5.34 (s, 2H) 2.39 (s, 3H). MS (M+H⁺)=395.

### Example 66

### N-[5-(1,4-dimethyl-1H-imidazol-2-yl)-2-methylphenyl]-4-{[3-(2-morpholin-4-ylethoxy)benzyl]oxy}benzamide

### 66a. methyl 4-(3-(2-morpholinoethoxy)benzyloxy)benzoate

In a 50 mL round-bottomed flask was added (3-(2-morpholinoethoxy)phenyl)methanol (0.873 g, 3.68 mmol) and 4-methylbenzene-1-sulfonyl chloride (0.701 g, 3.68 mmol) in DCM (10 mL) to give a colorless solution. To the mixture was added TEA (1.026 mL, 7.36 mmol) and DMAP (catalytic). After the reaction was stirred RT for 3 h sat. NH₄Cl (20 mL) was added. The aqueous layer was extracted with DCM (2×10 mL) and the combine organic layers were dried (Na₂SO₄) and concentrated in vacuo to afford 4-(2-(3-(chloromethyl)phenoxy)ethyl)morpholine. In a 50 mL round-bottomed flask was added 4-(2-(3-(chloromethyl)phenoxy)ethyl)morpholine (0.767 g, 3.0 mmol) and methyl 4-hydroxybenzoate (0.456 g, 3.0 mmol) in MeCN (25 mL) to give a colorless solution. K₂CO₃ was added (1.05 g, 7.5 mmol). The reaction was stirred at 85° C. for 4 h. After concentration in vacuo, the residue was diluted with water (10 mL) and extracted with EtOAc (2×10 mL). The combined organic phase was dried (Na₂SO₄) and concentrated to give the crude product, which was purified by ISCO MPLC (10% MeOH/DCM) to give the title compound. ¹H NMR (CDCl₃) δ 2.69 (br s, 4H), 2.91 (br s, 2H), 3.82 (br s, 4H), 3.90 (m, 3H), 4.21 (br s, 2H), 5.11 (s, 2H), 6.89 (dd, 1H), 7.01 (m, 4H), 7.32 (t, 1H), 8.01 (m, 2H).

### 66b. 4-(3-(2-morpholinoethoxy)benzyloxy)benzoic acid hydrochloride

In a 50 mL round-bottomed flask was added methyl 4-(3-(2-morpholinoethoxy)benzyloxy)benzoate (0.297 g, 0.8 mmol) and LiOH (0.096 g, 4.0 mmol) in MeOH (10 mL) to give a colorless suspension. The reaction was stirred at 65° C. for 3 h. Concentration under reduced pressure was followed by the addition of water (5 mL). 1N HCl was added to adjust the pH to 1, and the precipitate was collected to give the title compound as a white solid. ¹H NMR (DMSO-d₆) δ 3.20 (d, 2H), 3.56 (br s, 4H), 3.77 (br s, 2H), 3.95 (br s, 2H), 4.41 (br s, 2H), 5.18 (s, 2H), 7.00 (br s, 1H), 7.10 (d, 4H), 7.36 (t, 1H), 7.90 (d, 2H), 12.67 (br s, 1H).

### 66c. N-(5-(1,4-dimethyl-1H-imidazol-2-yl)-2-methylphenyl)-4-(3-(2-morpholinoethoxy)benzyloxy)benzamide

In a 50 mL round-bottomed flask was dissolved 4-(3-(2-morpholinoethoxy)benzyloxy)benzoic acid hydrochloride (0.14 g, 0.36 mmol) in SOCl₂ (1 mL) to give a colorless solution. The reaction was stirred at RT for 1 h, and SOCl₂ was then removed under reduced pressure to give 4-{[3-(2-morpholin-4-ylethoxy)benzyl]oxy}benzoyl chloride. To the flask was added pyridine (5 mL) and 5-(1,4-dimethyl-1H-imidazol-2-yl)-2-methylaniline (0.072 g, 0.36 mmol). The mixture was stirred at RT for 1 h, and the solution was concentrated under reduced pressure. DCM (5 mL) and sat. NaHCO₃ (10 mL) were added. The aqueous layer was extracted with DCM (2×5 mL) and the combined organic layers were dried (Na₂SO₄), and concentrated to give the crude product which was purified by ISCO MPLC (10% MeOH/DCM) to give the title compound (22 mg, 12%). ¹H NMR (DMSO-d₆) δ 2.11 (s, 3H), 2.27 (s, 3H), 2.50 (br s, 4H), 2.69 (s, 2H), 3.57 (m, 4H), 3.69 (s, 3H), 4.10 (t, 5.19 (s, 2H), 6.93 (s, 2H), 7.05 (s, 2H), 7.14 (m, 2H), 7.32 (m, 2H), 7.45 (m, 1H), 7.65 (s, 1H), 7.97 (m, 2H), 9.80 (s, 1H). MS (M+H⁺)=541.

### Example 67

### N-[5-(1H-imidazol-2-yl)-2,4-dimethylphenyl]-4-(pyridin-2-ylmethoxy)benzamide

### 67a. N-(5-bromo-2,4-dimethylphenyl)-4-(pyridin-2-ylmethoxy))benzamide

In a round-bottomed flask was placed 5-bromo-2,4-dimethylaniline (5 g, 25 mmol), 4-(pyridin-2-ylmethoxy)benzoic acid (6.3 g, 26.5 mmol), and DIPEA (8.9 mL, 50 mmol) in DMF (50 mL). The mixture was cooled to 0° C. with a water-ice bath before HATU (11.5 g, 30 mmol) was added. The mixture was warmed to RT and stirred overnight. To the reaction solution was added water (200 mL). The precipitate was collected by filtration to afford the title compound (4 g, 41% yield). ¹H NMR (DMSO-d₆) δ 2.14 (s, 3H), 2.27 (s, 3H), 5.26 (s, 2H), 7.13 (d, 2H), 7.23 (s, 1H), 7.34 (m, 1H), 7.52 (t, 1H), 7.56 (s, 1H), 7.82 (m, 1H), 7.92 (d, 2H), 8.57 (m, 1H).

### 67b. N-(2,4-dimethyl-5-(4,4,5,5-tetramethyl-1,3,2-dioxaborolan-2-yl)phenyl)-4-(pyridin-2-ylmethoxy)benzamide

In a round-bottomed flask was added N-(5-bromo-2,4-dimethylphenyl)-4-(pyridin-2-ylmethoxy)benzamide (4 g, 9.73 mmol), bis(pinacolato)diboron (2.96 g, 11.6 mmol), and KOAc (2.86 g, 29.2 mmol) in dioxane (50 mL) to give a suspension. To the mixture was added PdCl₂(dppf) (400 mg). The reaction was stirred at 80° C. under a nitrogen atmosphere overnight. The reaction mixture was concentrated in vacuo and water (80 mL) was added. The mixture was extracted with EtOAc (2×30 mL) and the combined organic layers were dried (Na₂SO₄), then concentrated in vacuo to afford the crude product which was purified by ISCO MPLC (1% MeOH/DCM) to give the title compound (2.3 g, 51.7% yield). ¹H NMR (DMSO-d₆) δ 1.26 (s, 12H), 2.15 (s, 3H), 2.41 (s, 3H), 5.25 (s, 2H), 7.06 (s, 1H), 7.12 (m, 2H), 7.35 (m, 1H), 7.51 (m, 2H), 7.81 (m, 1H), 7.94 (m, 2H), 8.58 (m, 1H), 9.71 (s, 1H).

### 67c. N-[5-(1H-imidazol-2-yl)-2,4-dimethylphenyl]-4-(pyridin-2-ylmethoxy)benzamide

In a 10 mL vial was added N-(2,4-dimethyl-5-(4,4,5,5-tetramethyl-1,3,2-dioxaborolan-2-yl)phenyl)-4-(pyridin-2-ylmethoxy)benzamide (0.25 g, 0.55 mmol), 2-bromo-1H-imidazole (0.120 g, 0.82 mmol), and Cs₂CO₃ (0.444 g, 1.36 mmol) in dioxane (5 mL) to give a brown suspension. The reaction mixture was diluted with water (2 mL). Nitrogen was bubbled in for 20 min before Pd(PPh₃)₄ (0.063 g, 0.05 mmol) was added. The reaction was heated at 110° C. for 4 h under microwave conditions. The reaction mixture was concentrated under reduced pressure. The residue was purified by Gilson HPLC (MeCN/0.1% TFA in water). To the purified product was added HCl in Et₂O (0.5 mL, 1 mmol). The mixture was concentrated in vacuo to give the HCl salt of the title compound (10 mg, 4.2%). ¹H NMR (DMSO-d₆) δ 2.31 (s, 3H), 2.36 (s, 3H), 5.32 (s, 2H), 7.18 (d, 2H), 7.39 (s, 1H), 7.45 (br s, 1H), 7.61 (s, 2H), 7.84 (s, 2H), 7.96 (m, 3H), 8.63 (d, 1H), 9.90 (s, 1H), 14.54 (br s, 1H). MS (M+H⁺)=399.

The following Examples 68-73 were prepared in a similar fashion to Example 67 utilizing commercially available starting materials:

| | | | MS | |
|---|---|---|---|---|
| Ex. | Name | MW | (M + H⁺) | ¹H NMR (δ ppm) |
| 68 | N-[5-(1H-benzimidazol- | 448.52 | 449 | 2.35 (s, 3 H), 2.55 (s, 3 H), 5.37 (s, 2 H), |
| | 2-yl)-2,4- | | | 7.20 (d, 2 H), 7.46 (s, 1 H), 7.52 (m, 1 H), |
| | dimethylphenyl]-4- | | | 7.60 (m, 2 H), 7.68 (d, 1 H), 7.83 (s, 1 H), |
| | (pyridin-2- | | | 7.87 (m, 2 H), 8.01 (m, 3 H), 8.68 (d, 1 H), |
| | ylmethoxy)benzamide | | | 9.99 (s, 1 H) |
| 69 | N-[5-(1,5-dimethyl-1H- | 426.52 | 427 | 2.18 (s, 3 H), 2.33 (s, 3 H), 2.36 (s, 3 H), |
| | imidazol-2-yl)-2,4- | | | 3.54 (s, 3 H), 5.34 (s, 2 H), 7.18 (d, 2 H), |
| | dimethylphenyl]-4- | | | 7.41 (s, 1 H), 7.48 (m, 1 H), 7.56 (s, 1 H), |
| | (pyridin-2- | | | 7.60 (s, 1 H), 7.64 (d, 1 H), 7.98 (d, 3 H), |
| | ylmethoxy)benzamide | | | 8.66 (d, 1 H), 9.92 (s, 1 H), 14.47 (br s, 1 |
| | | | | H) |
| 70 | N-[5-(1,2-dimethyl-1H- | 426.52 | 427 | 2.16 (s, 3 H), 2.28 (s, 3 H), 2.62 (m, 3 H), |
| | imidazol-5-yl)-2,4- | | | 3.46 (s, 3 H), 5.33 (s, 2 H), 7.17 (d, 2 H), |
| | dimethylphenyl]-4- | | | 7.33 (d, 2 H), 7.47 (m, 1 H), 7.63 (m, 2 H), |
| | (pyridin-2- | | | 7.97 (m, 3 H), 8.65 (d, 1 H), 9.83 (s, 1 H), |
| | ylmethoxy)benzamide | | | 14.38 (br s, 1 H) |
| 71 | N-[2,4-dimethyl-5-(1- | 412.49 | 413 | 2.26 (s, 3 H), 2.37 (s, 3 H), 3.92 (s, 3 H), |
| | methyl-1H-imidazol-4- | | | 5.37 (s, 2 H), 7.18 (d, 2 H), 7.30 (s, 1 H), |
| | yl)phenyl]-4-(pyridin-2- | | | 7.53 (m, 2 H), 7.69 (d, 1 H), 7.91 (s, 1 H), |
| | ylmethoxy)benzamide | | | 8.02 (m, 3 H), 8.69 (d, 1 H), 9.21 (s, 1 H), |
| | | | | 9.87 (s, 1 H) |
| 72 | N-[5-(1H-imidazol-4-yl)- | 398.46 | 399 | 2.26 (s, 3 H), 2.36 (s, 3 H), 5.33 (s, 2 H), |
| | 2,4-dimethylphenyl]-4- | | | 7.16 (s, 1 H), 7.19 (s, 1 H), 7.30 (s, 1 H), |
| | (pyridin-2- | | | 7.48 (m, 2 H), 7.63 (d, 1 H), 7.87 (s, 1 H), |
| | ylmethoxy)benzamide | | | 7.97 (m, 3 H), 8.65 (d, 1 H), 9.24 (s, 1 H), |
| | | | | 9.85 (s, 1 H), 14.67 (br s, 1 H) |
| 73 | N-(5-(1,2-dimethyl-1H- | 426.51 | 427 | 2.26 (s, 3 H), 2.38 (s, 3 H), 2.61 (s, 3 H), |
| | imidazol-4-yl)-2,4- | | | 3.79 (s, 3 H), 5.30 (s, 2 H), 7.17 (m, 2 H), |
| | dimethylphenyl)-4- | | | 7.30 (s, 1 H), 7.41 (dd, 1 H), 7.52 (s, 1 H), |
| | (pyridin-2- | | | 7.58 (d, 1 H), 7.81 (s, 1 H), 7.90 (td, 1 H), |
| | ylmethoxy)benzamide | | | 7.97 (m, 2 H), 8.62 (d, 1 H), 9.82 (s, 1 H), |
| | | | | 14.24 (br s, 1 H) |

### Example 74

### N-[4-chloro-2-methyl-5-(1-methyl-1H-imidazol-2-yl)phenyl]-4-(pyridin-2-ylmethoxy)benzamide

### 74a. 1-bromo-2-chloro-4-methyl-5-nitrobenzene

In a 50-mL round-bottomed flask was placed 1-bromo-2-chloro-4-methylbenzene (2 g, 9.7 mmol) and concentrated H₂SO₄ (6.5 mL). The mixture was cooled to −20° C. and HNO₃ (1.5 mL) was added slowly over 5 min. To the reaction mixture was added ice water (15 g) after the addition of HNO₃. After allowing to warm to RT, the reaction mixture was extracted with EtOAc (2×10 mL). After drying (Na₂SO₄) the combined organic layers were concentrated in vacuo and the crude product was purified by ISCO MPLC (petroleum ether) to afford the title compound (1.3 g) in 62% yield. ¹H NMR (CDCl₃) δ 2.56 (s, 3H), 7.44 (s, 1H), 8.28 (s, 1H).

### 74b. 5-bromo-4-chloro-2-methylaniline

In a 200-mL round-bottomed flask was placed 1-bromo-2-chloro-4-methyl-5-nitrobenzene (4 g, 16 mmol) and FeCl₃ in silica gel (5%, 11.2 g) in MeOH (50 mL). The reaction mixture was heated to 70° C. for 15 min and then hydrazine monohydrate (8.8 mL, 192 mmol) was slowly added and the reaction mixture was refluxed overnight. After cooled to RT, the mixture was filtered, and concentrated in vacuo to afford the title compound (3.4 g) in 95% yield. ¹H NMR (DMSO-d₆) δ 1.98 (s, 3H), 6.90 (s, 1H), 7.10 (s, 1H).

### 74c. N-(5-bromo-4-chloro-2-methylphenyl)-4-(pyridin-2-ylmethoxy)benzamide

In a 100-mL round-bottomed flask was dissolved 4-(pyridin-2-ylmethoxy)benzoic acid (800 mg, 4 mmol) in SOCl₂ (6 mL). The solution was stirred for 1 h at RT. The solution was concentrated in vacuo to give 4-(pyridin-2-ylmethoxy)benzoyl chloride. The crude product was dissolved in DCM (10 mL) followed by the addition of 5-bromo-4-chloro-2-methylaniline (500 mg, 2.27 mmol), pyridine (5 mL), and TEA (10 mL). The reaction mixture was heated to 50° C. and stirred for 2 h. The mixture was concentrated in vacuo and the crude product was purified by ISCO MPLC (20-33% EtOAc/petroleum ether) to afford the title compound (270 mg) in 28% yield. ¹H NMR (DMSO-d₆) δ 2.05 (s, 3H), 2.25 (s, 2H), 7.13 (m, 2H), 7.34 (m, 1H), 7.54 (m, 2H), 7.77 (s, 1H), 7.84 (m, 1H), 7.92 (m, 2H), 8.58 (m, 1H), 9.81 (s, 1H).

### 74d. N-(4-chloro-2-methyl-5-(4,4,5,5-tetramethyl-1,3,2-dioxaborolan-2-yl)phenyl)-4-(pyridin-2-ylmethoxy)benzamide

In a 10 mL vial was added 4,4,4′,4′,5,5,5′,5′-octamethyl-2,2′-bi(1,3,2-dioxaborolane) (0.441 g, 1.74 mmol), N-(5-bromo-4-chloro-2-methylphenyl)-4-(pyridin-2-ylmethoxy)benzamide (0.5 g, 1.16 mmol), and KOAc (0.341 g, 3.47 mmol) in dioxane (80 mL) to give a colorless suspension. Nitrogen was bubbled in for 20 min before Pd(PPh₃)₄(0.134 g, 0.12 mmol) was added. The reaction was stirred at 115° C. in a microwave for 5 h. After concentration under reduced pressure, the crude product was purified by ISCO MPLC (0-5% MeOH in DCM) to give the title compound. ¹H NMR (DMSO-d₆) δ 1.07 (s, 6H), 1.16 (s, 6H), 2.22 (s, 3H), 5.28 (s, 2H), 7.16 (d, 2H), 7.36 (s, 1H), 7.54 (d, 1H), 7.59 (s, 1H), 7.63 (m, 1H), 7.85 (m, 1H), 7.96 (m, 2H), 8.60 (d, 1H), 9.82 (s, 1H).

### 74e. N-[4-chloro-2-methyl-5-(1-methyl-1H-imidazol-2-yl)phenyl]-4-(pyridin-2-ylmethoxy)benzamide

In a 10 mL vial was added N-(4-chloro-2-methyl-5-(4,4,5,5-tetramethyl-1,3,2-dioxaborolan-2-yl)phenyl)-4-(pyridin-2-ylmethoxy)benzamide (0.15 g, 0.31 mmol), 2-bromo-1-methyl-1H-imidazole (0.076 g, 0.47 mmol), and KOAc (0.077 g, 0.78 mmol) in dioxane (3.0 mL) to give a brown suspension. The reaction mixture was diluted with water (1.0 mL). Nitrogen was bubbled in for 20 min before Pd(PPh₃)₄ (0.036 g, 0.03 mmol) was added. The reaction was heated using a microwave at 130° C. for 2 h. After concentration in vacuo, the residue was diluted with MeOH (0.5 mL) and DMSO (0.5 mL). The solution was filtered and purified by Gilson HPLC (5-80% MeCN/0.1% TFA in water) to give the title compound (0.019 g, 14% yield). ¹H NMR (DMSO-d₆) δ 2.38 (s, 3H), 3.72 (s, 3H), 5.33 (s, 2H), 7.19 (d, 2H), 7.46 (m, 1H), 7.62 (d, 1H), 7.76 (s, 1H), 7.95 (m, 6H), 8.65 (d, 1H), 10.04 (s, 1H). MS (M+H⁺)=433.

The following Examples 75-77 were prepared in a similar fashion to Example 83 utilizing commercially available starting materials:

| | | | MS | |
|---|---|---|---|---|
| Ex. | Name | MW | (M + H⁺) | ¹H NMR (δ ppm) |
| 75 | N-[4-chloro-2-methyl- | 432.91 | 433 | 2.31 (s, 3 H), 3.92 (s, 3 H), 5.34 (s, 2 H), |
| | 5-(1-methyl-1H- | | | 7.19 (d, 2 H), 7.44-7.52 (m, 1 H), |
| | imidazol-4-yl)phenyl]- | | | 7.60-7.68 (m, 2 H), 7.78 (s, 1 H), |
| | 4-(pyridin-2- | | | 7.94-8.04 (m, 3 H), 8.12 (s, 1 H), 8.66 (d, 1 H), |
| | ylmethoxy)benzamide | | | 9.19 (s, 1 H), 9.98 (s, 1 H) |
| 76 | N-[4-chloro-5-(1H- | 418.88 | 419 | 2.37 (s, 3 H), 5.34 (s, 2 H), 7.20 (d, 2 H), |
| | imidazol-2-yl)-2- | | | 7.43-7.51 (m, 1 H), 7.63 (d, 1 H), |
| | methylphenyl]-4- | | | 7.73 (s, 1 H), 7.86-7.91 (m, 3 H), |
| | (pyridin-2- | | | 7.93-8.04 (m, 3 H), 8.66 (d, 1 H), 10.04 (s, 1 H), |
| | ylmethoxy)benzamide | | | 14.85 (br s, 2 H) |
| 77 | N-[4-chloro-5-(1,2- | 446.94 | 447 | 2.30 (s, 3 H), 2.63 (s, 3 H), 3.80 (s, 3 H), |
| | dimethyl-1H-imidazol- | | | 5.34 (s, 2 H), 7.19 (d, 2 H), 7.47 (m, 1 H), |
| | 4-yl)-2-methylphenyl]- | | | 7.63 (m, 2 H), 7.77 (s, 1 H), 7.97 (m, 3 |
| | 4-(pyridin-2- | | | H), 8.07 (s, 1 H), 8.65 (d, 1 H), 9.98 (s, 1 |
| | ylmethoxy)benzamide | | | H) |

### Example 78

### N-{5-[5-(hydroxymethyl)-1-methyl-1H-imidazol-4-yl]-2-methylphenyl}-4-(pyridin-2-ylmethoxy)benzamide

### 78a. N-(5-(5-formyl-1-methyl-1H-imidazol-4-yl)-2-methylphenyl)-4-(pyridin-2-ylmethoxy)benzamide

In a 10-mL vial was added 4-methyl-3-(4-(pyridin-2-ylmethoxy)benzamido)phenylboronic acid (0.5 g, 1.38 mmol), 4-bromo-1-methyl-1H-imidazole-5-carbaldehyde (0.326 g, 1.73 mmol), and K₂CO₃ (0.477 g, 3.45 mmol) in dioxane (3 mL) to give a white suspension. The reaction mixture was diluted with water (1.0 mL). Nitrogen gas was bubbled in for 20 min before Pd(PPh₃)₄ (0.160 g, 0.14 mmol) was added. The reaction was heated in a microwave oven at 130° C. for 2.5 h. The reaction was concentrated in vacuo and the residue was combined with MeOH (10 mL) and silica gel (2 g). The solvent was removed in vacuo, and the solid purified by ISCO MPLC (0-8% MeOH/DCM) to give the title compound. MS (M+H⁺)=427.

### 78b. N-{5-[5-(hydroxymethyl)-1-methyl-1H-imidazol-4-yl]-2-methylphenyl}-4-(pyridin-2-ylmethoxy)benzamide

In a 20 mL round-bottomed flask was dissolved N-(5-(5-formyl-1-methyl-1H-imidazol-4-yl)-2-methylphenyl)-4-(pyridin-2-ylmethoxy)benzamide (0.10 g, 0.23 mmol) in MeOH (2.0 mL) to give a colorless solution. The solution was cooled with an ice-water bath and cooled to 0° C. To the solution was added NaBH₄ (8.87 mg, 0.23 mmol). The reaction was stirred at 0° C. for 2 h. Water (0.5 mL) was added to the solution. After stirring for another 0.5 h and concentration in vacuo, the residue was diluted with MeOH (1 mL). The solution was filtered and purified by Gilson HPLC (5-80% MeCN/0.1% TFA in water) to give the title compound (0.057 g, 57% yield). ¹H NMR (DMSO-d₆) [symbol] 2.31 (s, 3H), 3.93 (s, 3H), 4.63 (s, 2H), 5.37 (s, 2H), 7.19 (d, 2H), 7.47 (m, 3H), 7.68 (m, 2H), 8.01 (m, 3H), 8.67 (m, 1H), 9.22 (s, 1H), 9.97 (s, 1H); MS (M+H⁺)=429.

The following Example 79 was prepared in a similar fashion to Example 78 utilizing commercially available starting materials:

| | | | MS | |
|---|---|---|---|---|
| Ex. | Name | MW | (M + H⁺) | ¹H NMR (δ ppm) |
| 79 | N-(5-(5-(hydroxymethyl)- | 442.51 | 443 | 2.21 (s, 3 H), 2.27 (s, 3 H), 3.93 (s, 3 H), |
| | 1-methyl-1H-imidazol-4- | | | 4.44 (s, 2 H), 5.32 (s, 2 H), 7.17 (d, 2 H), |
| | yl)-2,4-dimethylphenyl)-4- | | | 7.32 (d, 2 H), 7.45 (dd, 1 H), 7.61 (d, 1 |
| | (pyridin-2- | | | H), 7.95 (m, 3 H), 8.64 (d, 1 H), 9.21 (s, |
| | ylmethoxy)benzamide | | | 1 H), 9.85 (s, 1 H), 14.72 (br s, 1 H) |

### Example 80

### N-(2-methyl-5-{1-methyl-5-[(methylamino)methyl]-1H-imidazol-4-yl}phenyl)-4-(pyridin-2-ylmethoxy)benzamide

To a mixture of methanamine (0.32 mL, 2M in MeOH), (N-(5-(5-formyl-1-methyl-1H-imidazol-4-yl)-2-methylphenyl)-4-(pyridin-2-ylmethoxy)benzamide (0.18 g, 0.42 mmol) in MeOH (2 mL), and acetic acid (0.048 mL) was added sodium triacetoxyborohydride (0.313 g, 1.48 mmol) at RT. The reaction mixture was stirred at RT overnight. After concentration in vacuo, the crude product was purified using Gilson HPLC (MeCN/0.1% TFA in water) to yield the title compound (0.091 g, 49% yield). ¹H NMR (DMSO-d₆) [symbol] 2.34 (s, 3H), 2.55 (br s, 3H), 4.10 (s, 3H), 4.47 (br s, 2H), 5.42 (s, 2H), 7.21 (d, 2H), 7.50 (m, 2H), 7.61 (m, 1H), 7.77 (m, 2H), 8.10 (m, 3H), 8.73 (d, 1H), 9.35 (s, 1H), 9.80 (br s, 2H), 10.13 (s, 1H); MS (M+H⁺)=442.

### Example 81

### N-[2-methyl-5-(4-phenyl-1H-imidazol-2-yl)phenyl]-4-(pyridin-2-ylmethoxy)benzamide

### 81a. N-(5-cyano-2-methylphenyl)-4-(pyridin-2-ylmethoxy)benzamide

In a 250 mL round-bottomed flask was dissolved 4-(pyridin-2-ylmethoxy)benzoic acid hydrochloride (6.0 g, 22.58 mmol) in DCM (25 mL) to give a colorless solution. SOCl₂ (8.24 mL, 112.91 mmol) was added and the reaction was stirred at RT for 3 h. Evaporation in vacuo afforded 4-(pyridin-2-ylmethoxy)benzoyl chloride, which was diluted with pyridine (25 mL). To the reaction mixture was added 3-amino-4-methylbenzonitrile (2.98 g, 22.58 mmol) and the reaction was stirred at RT overnight. After concentration in vacuo, the solid residue was diluted with sat. NaHCO₃ (50 mL) and the mixture was stirred at RT for 0.5 h. The mixture was filtered and the solid was triturated with MeOH (50 mL) for 0.5 h. Filtration afforded the title compound. ¹H NMR (DMSO-d₆) δ 2.33 (s, 3H), 5.45 (s, 2H), 7.21 (m, 2H), 7.49 (d, 1H), 7.63 (dd, 1H), 7.68 (t, 1H), 7.83 (m, 2H), 8.01 (m, 2H), 8.22 (m, 1H), 8.77 (d, 1H), 10.00 (s, 1H).

### 81b. N-(5-carbamimidoyl-2-methylphenyl)-4-(pyridin-2-ylmethoxy)benzamide

In a 100 mL round-bottomed flask was added NH₄Cl (2.337 g, 43.68 mmol) in toluene (100 mL) to give a colorless suspension. The mixture was cooled to 0° C. before trimethylaluminum (21.84 mL, 2 M in toluene) was added dropwise. After the addition, the reaction was allowed to warm up to RT and the reaction was stirred for at RT for 2 h. N-(5-cyano-2-methylphenyl)-4-(pyridin-2-ylmethoxy)benzamide (3.0 g, 8.74 mmol) was added and the reaction was heated to 108° C. for 20 h. The reaction mixture was cooled to RT and then poured into silica gel (20 g) in chloroform (40 mL). The mixture was stirred for 10 min, and filtered. The filter cake was washed with MeOH (100 mL). The filtrate was concentrated, and DCM (100 mL) was added to the residue. Filtration afforded a white precipitate that was purified by ISCO MPLC (20% MeOH/DCM) to give the title compound. ¹H NMR (DMSO-d₆) 62.34 (s, 3H), 5.29 (s, 2H), 7.18 (m, 2H), 7.37 (m, 1H), 7.54 (m, 2H), 7.64 (dd, 1H), 7.86 (m, 2H), 8.00 (m, 2H), 8.60 (d, 1H), 9.05 (s, 2H), 9.33 (s, 2H), 10.00 (s, 1H). MS (M−H⁺)=359.

### 81c. N-[2-methyl-5-(4-phenyl-1H-imidazol-2-yl)phenyl]-4-(pyridin-2-ylmethoxy)benzamide

In a 20 mL vial was dissolved KHCO₃ (0.050 g, 0.50 mmol) in water (0.500 mL) to give a colorless solution. The reaction mixture was diluted with THF (2.0 mL). To the solution was added N-(5-carbamimidoyl-2-methylphenyl)-4-(pyridin-2-ylmethoxy)benzamide hydrochloride (0.1 g, 0.25 mmol). The reaction mixture was heated to 75° C. for 5 min, and then 2-bromo-1-phenylethanone (0.050 g, 0.25 mmol) in THF (1 mL) was slowly added over 5 min at 75° C. The reaction was stirred at 75° C. for 30 min. After cooling to RT, the solution was concentrated in vacuo and the crude product was purified by Gilson HPLC (5-85% MeCN/10 mM NH₄OAc in water) to give the title compound (0.018 g, 16% yield). ¹H NMR (DMSO-d₆) [symbol] 2.27 (br s, 3H), 5.30 (br s, 2H), 7.18 (d, 3H), 7.38 (br s, 4H), 7.56 (d, 1H), 7.75 (br s, 1H), 7.86 (d, 4H), 8.02 (d, 3H), 8.61 (s, 1H), 9.90 (s, 1H), 12.63 (br s, 1H). MS (M+H⁺)=461.

The following Examples 82-87 were prepared in a similar fashion to Example 81 utilizing commercially available starting materials:

| | | | MS | |
|---|---|---|---|---|
| Ex. | Name | MW | (M + H⁺) | ¹H NMR (δ ppm) |
| 82 | N-{2-methyl-5-[4-(1,3- | 467.55 | 468 | 2.30 (s, 3 H), 5.37 (s, 2 H), 7.20 (d, 2 H), |
| | thiazol-2-yl)-1H-imidazol- | | | 7.45 (d, 1 H), 7.53 (dd, 1 H), 7.69 (d, 1 |
| | 2-yl]phenyl}-4-(pyridin-2- | | | H), 7.74 (d, 1 H), 7.87 (dd, 1 H), 7.90 (d, |
| | ylmethoxy)benzamide | | | 1 H), 8.06 (m, 5 H), 8.70 (d, 1 H), |
| | | | | 9.96 (s, 1 H) |
| 83 | N-{2-methyl-5-[4-(4- | 529.64 | 530 | 1.98 (m, 4 H), 2.35 (s, 3 H), 3.30 (m, 4 |
| | pyrrolidin-1-ylphenyl)-1H- | | | H), 5.42 (s, 2 H), 6.66 (d, 2 H), 7.23 (d, |
| | imidazol-2-yl]phenyl}-4- | | | 2 H), 7.60 (m, 2 H), 7.77 (d, 3 H), |
| | (pyridin-2- | | | 8.04 (m, 4 H), 8.15 (m, 2 H), 8.74 (d, 1 H), |
| | ylmethoxy)benzamide | | | 10.08 (s, 1 H), 14.41 (br s, 1 H), |
| | | | | 14.88 (br s, 1 H) |
| 84 | N-[2-methyl-5-(4-pyridin- | 461.52 | 462 | 2.27 (s, 3 H), 5.29 (s, 2 H), 7.18 (d, 2 H), |
| | 3-yl-1H-imidazol-2- | | | 7.39 (m, 3 H), 7.55 (d, 1 H), 7.82 (d, 1 |
| | yl)phenyl]-4-(pyridin-2- | | | H), 7.87 (m, 2 H), 8.01 (m, 3 H), 8.18 (d, |
| | ylmethoxy)benzamide | | | 1 H), 8.41 (d, 1 H), 8.61 (d, 1 H), |
| | | | | 9.06 (d, 1 H), 9.90 (s, 1 H) |
| 85 | N-[5-(4-ethyl-1H-imidazol- | 412.49 | 413 | 1.19 (t, 3 H), 2.23 (s, 3 H), 2.50 (m, 2 |
| | 2-yl)-2-methylphenyl]-4- | | | H), 5.29 (s, 2 H), 6.80 (m, 1 H), 7.17 (d, |
| | (pyridin-2- | | | 2 H), 7.30 (d, 1 H), 7.37 (dd, 1 H), |
| | ylmethoxy)benzamide | | | 7.55 (d, 1 H), 7.68 (m, 1 H), 7.86 (m, 2 H), |
| | | | | 7.99 (d, 2 H), 8.60 (d, 1 H), 9.83 (s, 1 |
| | | | | H), 12.14 (br s, 1 H) |
| 86 | N-[5-(4-tert-butyl-1H- | 440.54 | 441 | 1.25 (s, 9 H), 2.23 (s, 3 H), 5.29 (s, 2 H), |
| | imidazol-2-yl)-2- | | | 6.86 (s, 1 H), 7.17 (m, 2 H), 7.31 (d, 1 |
| | methylphenyl]-4-(pyridin- | | | H), 7.37 (dd, 1 H), 7.55 (d, 1 H), |
| | 2-ylmethoxy)benzamide | | | 7.68 (m, 1 H), 7.86 (m, 2 H), 7.99 (m, 2 H), |
| | | | | 8.60 (d, 1 H), 9.87 (br s, 1 H), 12.09 (br |
| | | | | s, 1 H) |
| 87 | N-[5-(4-cyclopropyl-1H- | 424.50 | 425 | 0.86 (m, 2 H), 1.02 (m, 2 H), 2.00 (m, 1 |
| | imidazol-2-yl)-2- | | | H), 2.33 (s, 3 H), 5.36 (s, 2 H), 7.21 (d, 2 |
| | methylphenyl]-4-(pyridin- | | | H), 7.53 (m, 3 H), 7.68 (d, 1 H), |
| | 2-ylmethoxy)benzamide | | | 7.89 (dd, 1 H), 8.03 (d, 3 H), 8.09 (s, 1 H), |
| | | | | 8.68 (d, 1 H), 10.01 (s, 1 H), 14.58 (br s, |
| | | | | 2 H) |

### Example 88

### N-{5-[4-(hydroxymethyl)-1H-imidazol-2-yl]-2-methylphenyl}-4-(pyridin-2-ylmethoxy)benzamide

In a 10-mL vial was added N-(5-carbamimidoyl-2-methylphenyl)-4-(pyridin-2-ylmethoxy)benzamide hydrochloride (0.15 g, 0.38 mmol), dihydroxyacetone (0.170 g, 1.89 mmol), and NH₄OH (2 mL) to give a yellow suspension. The reaction mixture was diluted with THF (2 mL) and the mixture became clear. The reaction was heated to 80° C. for 1.5 h. After removal of the solvents under reduced pressure, the crude product was purified by Gilson HPLC (5-75% MeCN/0.1% TFA in water) to give the title compound (0.072 g, 46% yield). ¹H NMR (DMSO-d₆) [symbol] 2.34 (s, 3H), 4.57 (s, 2H), 5.36 (s, 2H), 7.21 (d, 2H), 7.50 (m, 1H), 7.56 (d, 1H), 7.66 (m, 2H), 7.90 (d, 1H), 8.01 (m, 3H), 8.15 (s, 1H), 8.67 (d, 1H), 10.01 (s, 1H), 14.72 (m, 2H). MS (M+H⁺)=415.

### Example 89

### N-[5-(1H-imidazol-2-yl)-2-methylphenyl]-4-[(4-morpholin-4-ylpyridin-2-yl)methoxy]benzamide

### 89a. 4-((4-chloropyridin-2-yl)methoxy)benzoic acid

In a 50-mL round-bottomed flask was dissolved (4-chloropyridin-2-yl)methanol (4.6 g, 32.04 mmol) and tosyl chloride (6.72 g, 35.24 mmol) in DCM (10 mL) to give a colorless solution. To the mixture was added TEA (8.93 mL, 64.08 mmol) and DMAP (0.05 g). The reaction was stirred at RT for 0.5 h, and washed with sat NH₄Cl (20 mL). The organic layer was dried (Na₂SO₄), filtered, and concentrated to give crude (4-chloropyridin-2-yl)methyl 4-methylbenzenesulfonate. To this product was added methyl 4-hydroxybenzoate (3.07 g, 20.15 mmol), K₂CO₃ (11.14 g, 80.60 mmol), and MeCN (100 mL). The reaction was stirred at 80° C. for 4 h. The solvent was removed under reduced pressure, and to the residue was added water (50 mL) and EtOAc (100 mL). The aqueous layer was extracted with EtOAc (2×50 mL), and the combined organic layers were dried (Na₂SO₄), and concentrated to give crude methyl 4-((4-chloropyridin-2-yl)methoxy)benzoate. To this material was added LiOH (0.828 g, 34.57 mmol) and MeOH (100 mL). The reaction mixture was heated to 70° C. overnight and the solvent was removed under reduced pressure. The residue was diluted with water (50 mL) and concentrated HCl (12N) was added dropwise to adjust the pH to 1. The precipitate was collected by filtration to yield the title compound. ¹H NMR (DMSO-d₆) [symbol] 5.28 (s, 2H), 7.14 (d, 2H), 7.54 (dd, 1H), 7.66 (d, 1H), 7.91 (d, 2H), 8.58 (d, 1H), 12.69 (br s, 1 H).

### 89b. 5-(1H-imidazol-2-yl)-2-methylaniline

In a 10 mL vial was added 2-bromo-1H-imidazole (1.891 g, 12.87 mmol), 2-methyl-5-(4,4,5,5-tetramethyl-1,3,2-dioxaborolan-2-yl)aniline (2.0 g, 8.58 mmol), and Cs₂CO₃ (5.6 g, 17 mmol) in dioxane (6.0 mL) to give a colorless suspension. The reaction mixture was diluted with water (1.5 mL). After bubbling in nitrogen for 20 min, Pd(PPh₃)₄ (1.487 g, 1.29 mmol) was added. The reaction was heated at 110° C. in a microwave oven for 50 h. The solvents were removed under reduced pressure, and the residue was purified by ISCO MPLC (10% MeOH/DCM) to give the title compound. MS (M+H⁺)=174.

### 89c. N-(5-(1H-imidazol-2-yl)-2-methylphenyl)-4-((4-chloropyridin-2-yl)methoxy)benzamide

In a 50-mL round-bottomed flask was added 4-((4-chloropyridin-2-yl)methoxy)benzoic acid (1.522 g, 5.77 mmol) in SOCl₂ (10 mL) to give a colorless suspension. The reaction was stirred at RT for 2 h and the mixture became clear. After concentrating in vacuo, pyridine (15 mL) and 5-(1H-imidazol-2-yl)-2-methylaniline (1.0 g, 5.77 mmol) was added to the residue. After stirring at RT for 0.5 h, the reaction was heated at 65° C. for 2 h. After concentrating in vacuo, to the residue was added sat. NaHCO₃ (5 mL) and the solution was extracted with DCM (2×10 mL). The organic phases were combined, dried (Na₂SO₄), and concentrated in vacuo. The crude product was purified by ISCO MPLC (10% MeOH/DCM) to give the title compound. MS (M+H⁺)=419.

### 89d. N-[5-(1H-imidazol-2-yl)-2-methylphenyl]-4-[(4-morpholin-4-ylpyridin-2-yl)methoxy]benzamide

In a 10 mL vial was dissolved N-(5-(1H-imidazol-2-yl)-2-methylphenyl)-4-((4-chloropyridin-2-yl)methoxy)benzamide (0.02 g, 0.05 mmol) and morpholine (0.017 g, 0.19 mmol) to give a colorless solution. The reaction was stirred at 160° C. for 3 h under microwave conditions. After the reaction mixture was cooled to RT, the mixture was concentrated in vacuo and the residue purified by Gilson HPLC (MeCN/0.1% TFA in water) to give the title compound (0.020 g, 89% yield). ¹H NMR (DMSO-d₆) [symbol] 2.34 (s, 3H), 3.74 (d, 8H), 5.34 (s, 2H), 7.22 (m, 3H), 7.47 (d, 1H), 7.57 (d, 1H), 7.80 (s, 2H), 7.93 (s, 1H), 8.08 (d, 2H), 8.14 (s, 1H), 8.32 (d, 1H), 10.11 (s, 1H), 14.87 (br s, 1H). MS (M+H⁺)=470.

The following Examples 90-91 were prepared in a similar fashion to Example 99 utilizing commercially available starting materials:

| | | | MS | |
|---|---|---|---|---|
| Ex. | Name | MW | (M + H⁺) | ¹H NMR (δ ppm) |
| 90 | 4-({4-[4-(2- | 512.61 | 513 | 2.33 (s, 3 H), 2.65 (s, 3 H), 3.68 (s, 3 H), |
| | hydroxyethyl)piperazin- | | | 5.37 (s, 2 H), 7.19 (d, 2 H), 7.32 (m, 1 H), |
| | 1-yl]pyridin-2- | | | 7.49 (m, 2 H), 7.57 (s, 1 H), 7.68 (d, 1 H), |
| | yl}methoxy)-N-[5-(1H- | | | 7.74 (s, 1 H), 8.01 (d, 3 H), 8.67 (br s, 1 H), |
| | imidazol-2-yl)-2- | | | 9.94 (s, 1 H), 14.47 (br s, 1 H) |
| | methylphenyl]benzamide | | | |
| 91 | 4-[(4-chloropyridin-2- | 432.91 | 433 | 2.34 (s, 3 H), 3.81 (s, 3 H), 5.36 (s, 2 H), |
| | yl)methoxy]-N-[2- | | | 7.02 (s, 1 H), 7.24 (d, 2 H), 7.29 (s, 1 H), |
| | methyl-5-(1-methyl-1H- | | | 7.42 (d, 1 H), 7.53 (d, 1 H), 7.59 (dd, 1 H), |
| | imidazol-2- | | | 7.72 (s, 2 H), 8.04 (d, 2 H), 8.64 (d, 1 H), |
| | yl)phenyl]benzamide | | | 9.90 (s, 1 H) |

### Example 92

### N-[5-(1H-imidazol-2-yl)-2-methylphenyl]-4-[(4-methoxypyridin-2-yl)methoxy]benzamide

In a 10 mL vial was dissolved N-(5-(1H-imidazol-2-yl)-2-methylphenyl)-4-((4-chloropyridin-2-yl)methoxy)benzamide (0.09 g, 0.21 mmol) and sodium methoxide (4.30 mL, 2.15 mmol) to give a colorless solution. The reaction was stirred at 140° C. for 1 h using a microwave reactor. After the reaction mixture was cooled to RT, the reaction was concentrated in vacuo and the crude product was purified by Gilson HPLC (MeCN/0.1% TFA in water) to give the title compound (0.021 g, 24%). ¹H NMR (DMSO-d₆) [symbol] 2.35 (s, 3H), 4.02 (s, 3H), 5.43 (s, 2H), 7.24 (m, 2H), 7.36 (m, 1H), 7.50 (br s, 1H), 7.57 (d, 1H), 7.81 (s, 2H), 7.91 (d, 1H), 8.06 (m, 2H), 8.13 (s, 1H), 8.67 (d, 1H), 10.07 (s, 1H), 14.86 (br s, 1H). MS (M+H)=415.

The following Example 93 was prepared in a similar fashion to Example 92 utilizing commercially available starting materials:

| | | | MS | |
|---|---|---|---|---|
| Ex. | Name | MW | (M + H⁺) | ¹H NMR (δ ppm) |
| 93 | 4-[(4-methoxypyridin-2- | 428.49 | 429 | 2.28 (s, 3 H), 3.75 (s, 3 H), 3.83 (s, 3H), |
| | yl)methoxy]-N-[2-methyl- | | | 5.22 (s, 2 H), 6.95 (m, 2 H), 7.08 (m, 1 H), |
| | 5-(1-methyl-1H-imidazol- | | | 7.16 (m, 2 H), 7.24 (s, 1 H), 7.37 (d, 1 H), |
| | 2-yl)phenyl]benzamide | | | 7.46 (d, 1 H), 7.654 (s, 1 H), 7.98 (d, 2 H), |
| | | | | 8.40 (d, 1 H), 9.86 (s, 1 H) |

Alternatively, Examples 92-93 can be prepared in the following manner:

### Step A. N-(5-(1H-imidazol-2-yl)-2-methylphenyl)-4-((4-chloropyridin-2-yl)methoxy)benzamide

Prepared in a similar fashion to Example 119. MS (M+H⁺)=419.

### Step B. N-(5-(1H-imidazol-2-yl)-2-methylphenyl)-4-((4-methoxypyridin-2-yl)methoxy)benzamide

A mixture of N-(5-(1H-imidazol-2-yl)-2-methylphenyl)-4-((4-chloropyridin-2-yl)methoxy)benzamide (0.812 mL, 0.41 mmol) in 15 mL of 0.5M sodium methoxide in MeOH was stirred at 80° C. overnight. After concentrating in vacuo, the residue was purified with Gilson HPLC (5-55% MeCN/0.1% TFA in water) to yield the title compound as a white solid (90 mg, 49.2%).

### Example 94

### 4-({4-[2-(dimethylamino)ethoxy]pyridin-2-yl}methoxy)-N-[5-(1H-imidazol-2-yl)-2-methylphenyl]benzamide

In a 10 mL vial was dissolved N-(5-(1H-imidazol-2-yl)-2-methylphenyl)-4-((4-chloropyridin-2-yl)methoxy)benzamide (0.10 g, 0.24 mmol), 2-(dimethylamino)ethanol (0.128 g, 1.43 mmol), and potassium tert-butoxide (0.321 g, 2.86 mmol) in t-butanol (3 mL) to give a colorless suspension. The reaction was heated at 110° C. under microwave for 2 h. After cooled to RT, the mixture was concentrated in vacuo and the crude product was purified by Gilson HPLC (MeCN/0.1% TFA in water) to give the title compound (0.027 g, 24% yield). ¹H NMR (DMSO-d₆) [symbol] 2.34 (s, 3H), 2.85 (d, 6H), 3.56 (d, 2H), 4.61 (br s, 2H), 5.40 (s, 2H), 7.23 (d, 2H), 7.30 (br s, 1H), 7.44 (br s, 1H), 7.57 (d, 1H), 7.80 (s, 2H), 7.94 (d, 1H), 8.06 (d, 2H), 8.14 (s, 1H), 8.65 (br s, 1H), 10.09 (s, 1H), 10.63 (br s, 1H), 14.88 (br s, 1H). MS (M+H⁺)=472.

The following Examples 95-96 were prepared in a similar fashion to Example 94 utilizing commercially available starting materials:

| | | | MS | |
|---|---|---|---|---|
| Ex. | Name | MW | (M + H⁺) | ¹H NMR (δ ppm) |
| 95 | N-[5-(1H-imidazol-2- | 511.62 | 512 | 1.39 (d, 1 H), 1.71 (d, 1 H), 1.80 (br s, 4 H), |
| | yl)-2-methylphenyl]- | | | 2.34 (s, 3 H), 3.00 (m, 2 H), 3.51 (m, 4 H), |
| | 4-{[4-(2-piperidin-1- | | | 4.65 (br s, 2 H), 5.39 (s, 2 H), 7.23 (d, 2 H), |
| | ylethoxy)pyridin-2- | | | 7.30 (br s, 1 H), 7.43 (br s, 1 H), 7.57 (d, 1 |
| | yl]methoxy}benzamide | | | H), 7.81 (s, 2 H), 7.92 (d, 1 H), 8.06 (d, 2 H), |
| | | | | 8.13 (s, 1 H), 8.64 (br s, 1 H), 10.08 (s, 1 H), |
| | | | | 10.57 (br s, 1 H), 14.88 (br s, 1 H) |
| 96 | N-[2-methyl-5-(1- | 490.56 | 491 | 2.26 (S, 3 H), 3.73 (s, 3 H), 5.22 (s, 2 H), |
| | methyl-1H-imidazol- | | | 6.83 (m, 1 H), 6.94 (s, 1 H), 7.03 (d, 1 H), 7.12 (m, |
| | 2-yl)phenyl]-4-[(4- | | | 4 H), 7.22 (s, 1 H), 7.31 (m, 2 H), 7.46 (m, 3 |
| | phenoxypyridin-2- | | | H), 7.63 (s, 1 H), 7.95 (d, 2 H), 8.45 (d, 1 H), |
| | yl)methoxy]benzamide | | | 9.84 (s, 1 H) |

### Example 97

### 4-[(4-ethoxypyridin-2-yl)methoxy]-N-[2-methyl-5-(1-methyl-1H-imidazol-2-yl)phenyl]benzamide

### 97a 3-(4-(benzyloxy)benzamido)-4-methylphenylboronic acid

The title compound was prepared in a fashion similar to the preparation of Example 1, step c, utilizing commercial available reagents. ¹H NMR (d₃-MeOD) [symbol] 2.31 (s, 3H), 5.20 (s, 2 H), 7.13 (d, 2H), 7.37 (m, 4H), 7.48 (m, 3H), 7.58 (s, 1H), 7.95 (s, 2H). MS (M−H⁺)=360.

### 97b. 4-(benzyloxy)-N-(2-methyl-5-(4,4,5,5-tetramethyl-1,3,2-dioxaborolan-2-yl)phenyl)benzamide

In a 200-mL round-bottomed flask was placed 3-(4-(benzyloxy)benzamido)-4-methylphenylboronic acid (4.0 g, 11.1 mmol) in THF (50 mL), 4,4,4′,4′,5,5′,5′,5′-octamethyl-2,2′-bi(1,3,2-dioxaborolane) (2.0 g, 7.9 mmol) and Cs₂CO₃ (9 g, 27 mmol) were added. After nitrogen was bubbled in for 20 min, Pd(PPh₃)₄ (0.5 g) was added and the mixture was refluxed at 110° C. for 5 h. After concentration in vacuo, the residue was purified by ISCO MPLC (10% MeOH/DCM) to give the title compound. ¹H NMR (DMSO-d₆) [symbol] 1.26 (s, 12H), 2.22 (s, 3H), 5.18 (s, 2H), 7.12 (d, 2H), 7.23-7.47 (m, 7H), 7.62 (s, 1H), 7.93 (d, 2H), 9.72 (s, 1H).

### 97c. 4-(benzyloxy)-N-(2-methyl-5-(1-methyl-1H-imidazol-2-yl)phenyl)benzamide

In a 200-mL round-bottomed flask was placed 4-(benzyloxy)-N-(2-methyl-5-(4,4,5,5-tetramethyl-1,3,2-dioxaborolan-2-yl)phenyl)benzamide (4.0 g), 2-bromo-1-methyl-1H-imidazole (2.91 g), Cs₂CO₃ (7.35 g, 22.6 mmol), and Pd(PPh₃)₄ in dioxane (100 mL) and water (50 mL). The mixture was stirred at 100° C. overnight under a nitrogen atmosphere. After cooling to RT, the reaction mixture was concentrated under reduced pressure. The residue was pre-absorbed on silica gel (20 g) and purified by ISCO MPLC (10% MeOH/DCM) to give the title compound (3.5 g, 88% yield). ¹H NMR (CDCl₃) [symbol] 2.16 (s, 3H), 3.72 (s, 3H), 5.13 (s, 2H), 6.96 (s, 1H), 7.06-7.46 (m, 10H), 7.59 (s, 1H), 8.5 (d, 2H), 9.09 (s, 1H).

### 97d. 4-hydroxy-N-(2-methyl-5-(1-methyl-1H-imidazol-2-yl)phenyl)benzamide

In a 200-mL pressure vessel was dissolved 4-(benzyloxy)-N-(2-methyl-5-(1-methyl-1H-imidazol-2-yl)phenyl)benzamide (3.5 g, 8.82 mmol) in MeOH (100 mL). To the solution was added Pd/C (0.4 g, wet 10%). The was stirred at RT under H₂ (50 psi) atmosphere overnight. Filtration and concentration afforded the title compound (2.6 g) as a white solid. ¹H NMR (DMSO-d₆) [symbol] 2.28 (s, 3H), 3.76 (s, 3H), 6.87 (dd, 2H), 6.97 (s, 1H), 7.25 (s, 1H), 7.36 (d, 1H), 7.47 (m, 1H), 7.66 (s, 1H), 7.88 (d, 2H), 9.72 (s, 1H), 10.11 (s, 1H). MS (M+H⁺)=308.

### 97e. 4-[(4-ethoxypyridin-2-yl)methoxy]-N-[2-methyl-5-(1-methyl-1H-imidazol-2-yl)phenyl]benzamide

In a 10 mL vial was placed 4-hydroxy-N-(2-methyl-5-(1-methyl-1H-imidazol-2-yl)phenyl)benzamide (0.2 g, 0.65 mmol), 2-(chloromethyl)-4-ethoxypyridine (0.112 g, 0.65 mmol), and K₂CO₃ (0.360 g, 2.60 mmol) in MeCN (5 mL) to give a brown suspension. To the solution was added water (1 mL) and the reaction was stirred at 75° C. overnight. After the reaction was cooled to RT, the mixture was concentrated in vacuo and purified by Gilson HPLC (MeCN/10 mM NH₄OAc in water) to give the title compound (0.045 g, 16%). ¹H NMR (DMSO-d₆) [symbol] 1.40 (t, 3H), 2.37 (s, 3H), 3.89 (s, 3H), 4.35 (q, 2H), 5.45 (s, 2H), 7.23 (m, 2H), 7.40 (br s, 1H), 7.59 (m, 3H), 7.83 (m, 3H), 8.05 (m, 2H), 8.69 (d, 1H), 10.09 (s, 1H). MS (M+H⁺)=443.

The following Examples 98-101 were prepared in a similar fashion to Example 97 utilizing commercially available starting materials:

| | | | MS | |
|---|---|---|---|---|
| Ex. | Name | MW | (M + H⁺) | ¹H NMR (δ ppm) |
| 98 | 4-{[4- | 468.55 | 469 | 0.34 (m, 2 H), 0.58 (m, 2 H), 1.22 (m, 1 H), |
| | (cyclopropylmethoxy)pyridin- | | | 2.28 (s, 3 H), 3.76 (s, 3 H), 3.92 (d, 2 H), |
| | 2-yl]methoxy}-N-[2- | | | 5.21 (s, 2 H), 6.92 (m, 1 H), 6.96 (s, 1 H), |
| | methyl-5-(1-methyl-1H- | | | 7.06 (d, 1 H), 7.16 (m, 2 H), 7.24 (s, 1 H), |
| | imidazol-2- | | | 7.36 (m, 1 H), 7.47 (dd, 1 H), 7.66 (s, 1 H), |
| | yl)phenyl]benzamide | | | 7.98 (m, 2 H), 8.38 (d, 1 H), 9.83 (s, 1 H) |
| 99 | 4-[(4-bromo-2- | 501.38 | 502 | 9.78 (s, 1 H), 8.20 (d, 1 H), 7.99 (d, 1 H), |
| | cyanobenzyl)oxy]-N-[2- | | | 7.82-7.93 (m, 4 H), 7.77 (d, 1 H), 7.53 (d, 1 |
| | methyl-5-(1-methyl-1H- | | | H), 7.43 (dd, 1 H), 7.18 (d, 1 H), 6.88 (d, 2 |
| | imidazol-2- | | | H), 5.52 (s, 2 H), 3.75 (s, 3H), 2.36 (s, 3 H) |
| | yl)phenyl]benzamide | | | |
| 100 | 4-[(2-cyano-4- | 440.48 | 441 | 14.80 (br s, 1 H), 10.04 (s, 1 H), 8.04 (m, 2 |
| | fluorobenzyl)oxy]-N-[2- | | | H), 7.98 (dd, 1 H), 7.76-7.90 (m, 4 H), |
| | methyl-5-(1-methyl-1H- | | | 7.63-7.75 (m, 1 H), 7.59 (s, 2 H), 7.22 (m, 2 H), |
| | imidazol-2- | | | 5.34 (s, 2 H), 3.90 (s, 3 H), 2.38 (s, 3 H) |
| | yl)phenyl]benzamide | | | |
| 101 | 4-[(2-cyano-5- | 440.48 | 441 | 14.84 (br s, 1 H), 10.02 (s, 1 H), |
| | fluorobenzyl)oxy]-N-[2- | | | 7.91-8.11 (m, 3 H), 7.79 (dd, 2 H), 7.72 (d, 1 H), |
| | methyl-5-(1-methyl-1H- | | | 7.62 (dd, 1 H), 7.46-7.57 (m, 2 H), 7.42 (td, 1 |
| | imidazol-2- | | | H), 7.15 (d, 2 H), 5.30 (s, 2 H), 3.83 (s, 3 H), |
| | yl)phenyl]benzamide | | | 2.31 (s, 3 H) |

### Example 102

### N-[4-fluoro-5-(1H-imidazol-2-yl)-2-methylphenyl]-4-(pyridin-2-ylmethoxy)benzamide

### 102a. N-(5-bromo-4-fluoro-2-methylphenyl)-4-(pyridin-2-ylmethoxy)benzamide

In a 250 mL round-bottomed flask was placed 4-(pyridin-2-ylmethoxy)benzoic acid (3.4 g, 14.7) in DCM (50 mL) to give a suspension. To the solution, SOCl₂ (22.29 mL, 305.37 mmol) was added. The mixture was stirred at RT overnight. Concentration removed SOCl₂ and DCM to give crude 4-(pyridin-2-ylmethoxy)benzoyl chloride. To the residue was added 5-bromo-4-fluoro-2-methylaniline (3.0 g, 14.70 mmol), DIPEA (6.42 mL, 36.76 mmol), and DCM (60 mL) to give a black solution. The reaction was stirred at RT overnight. Concentration under reduced pressure gave the crude product, which was purified by ISCO MPLC (0-6% MeOH/DCM) to give the title compound. ¹H NMR (DMSO-d₆) [symbol] 2.21 (s, 3H), 5.28 (s, 2H), 7.16 (m, 2H), 7.36 (m, 2H), 7.54 (d, 1H), 7.66 (d, 1H), 7.85 (t, 1H), 7.95 (m, 2H), 8.60 (d, 1H), 9.83 (s, 1H). MS (M+H⁺)=416.

### 102b. N-(4-fluoro-2-methyl-5-(4,4,5,5-tetramethyl-1,3,2-dioxaborolan-2-yl)phenyl)-4-(pyridin-2-ylmethoxy)benzamide

In a 500 mL round-bottomed flask was combined 4,4,4′,4′,5,5,5′,5′-octamethyl-2,2′-bi(1,3,2-dioxaborolane) (1.376 g, 5.42 mmol), N-(5-bromo-4-fluoro-2-methylphenyl)-4-(pyridin-2-ylmethoxy)benzamide (1.5 g, 3.61 mmol), and KOAc (1.064 g, 10.84 mmol) in dioxane (80 mL) to give a colorless suspension. Nitrogen was bubbled in for 20 min before Pd(PPh₃)₄ (0.9 g, 0.78 mmol) was added. The reaction was stirred at 90° C. overnight. After concentration under reduced pressure, the crude product was purified by ISCO MPLC (10% MeOH/DCM) to give the title compound. MS (M+H⁺)=463.

### 102c. N-[4-fluoro-5-(1H-imidazol-2-yl)-2-methylphenyl]-4-(pyridin-2-ylmethoxy)benzamide

In a 10 mL vial was combined N-(4-fluoro-2-methyl-5-(4,4,5,5-tetramethyl-1,3,2-dioxaborolan-2-yl)phenyl)-4-(pyridin-2-ylmethoxy)benzamide (0.30 g, 0.65 mmol), 2-bromo-1H-imidazole (0.143 g, 0.97 mmol), and KOAc (0.159 g, 1.62 mmol) in dioxane (3 mL) to give a black suspension. Nitrogen gas was bubbled in for 20 min before Pd(PPh₃)₄(0.075 g, 0.06 mmol) was added. The reaction was heated under microwave at 130° C. for 4 h. After concentration under reduced pressure the residue was dissolved with DMSO (0.5 mL) and MeOH (1.5 mL), filtered and purified by Gilson HPLC (MeCN/0.1% TFA in water) to give the title compound (2 M HCl in Et₂O was added) as the HCl salt (0.050 g, 19% yield). ¹H NMR (DMSO-d₆) [symbol] 2.35 (s, 3H), 5.37 (s, 2H), 7.21 (d, 2H), 7.53 (m, 2H), 7.68 (d, 1H), 7.85 (s, 2H), 8.01 (m, 4H), 8.68 (d, 1H), 10.07 (s, 1H), 14.77 (br s, 1H). MS (M+H⁺)=403.

The following Examples 103-104 were prepared in a similar fashion to Example 102 utilizing commercially available starting materials:

| | | | MS | |
|---|---|---|---|---|
| Ex. | Name | MW | (M + H⁺) | ¹H NMR (δ ppm) |
| 103 | N-[4-fluoro-2-methyl-5- | 416.45 | 417 | 2.20 (s, 3 H), 2.31 (s, 3 H), 5.29 (s, 2 H), |
| | (2-methyl-1H-imidazol- | | | 7.14 (m, 3 H), 7.29 (br s, 1 H), 7.37 (m, 1 H), |
| | 4-yl)phenyl]-4-(pyridin- | | | 7.55 (d, 1 H), 7.87 (m, 2 H), 7.97 (d, 2 H), |
| | 2-ylmethoxy)benzamide | | | 8.60 (d, 1 H), 9.77 (s, 1 H), 11.97 (s, 1 H) |
| 104 | N-(5-(1,2-dimethyl-1H- | 430.49 | 431 | — |
| | imidazol-4-yl)-4-fluoro- | | | |
| | 2-methylphenyl)-4- | | | |
| | (pyridin-2- | | | |
| | ylmethoxy)benzamide | | | |

### Example 105

### N-[2-chloro-5-(1H-imidazol-2-yl)phenyl]-4-(pyridin-2-ylmethoxy)benzamide

### 105a. 4-chloro-3-(4-(pyridin-2-ylmethoxy)benzamido)phenylboronic acid

In a 250 mL round-bottomed flask was dissolved 3-amino-4-chlorophenylboronic acid (1.03 g, 6.01 mmol), 4-(pyridin-2-ylmethoxy)benzoic acid hydrochloride (1.597 g, 6.01 mmol), and DIPEA (2.099 mL, 12.02 mmol) in DMF (15 mL) to give a colorless solution. HATU (2.399 g, 6.31 mmol) was added at RT. The reaction was heated to 80° C. for 5 h. After cooling to RT, the reaction mixture was diluted with water (200 mL). The precipitate was collected by filtration and then washed with sat. NaHCO₃ (100 mL) to give the title compound. MS (M+H⁺)=383.

### 105b. N-[2-chloro-5-(1H-imidazol-2-yl)phenyl]-4-(pyridin-2-ylmethoxy)benzamide

In a 10 mL vial was combined 4-chloro-3-(4-(pyridin-2-ylmethoxy)benzamido)phenylboronic acid (0.17 g, 0.44 mmol), 2-bromo-1H-imidazole (0.131 g, 0.89 mmol), and KOAc (0.109 g, 1.11 mmol) in dioxane (4 mL) to give a colorless suspension. The reaction mixture was diluted with water (1.0 mL). Nitrogen gas was bubbled in for 20 min before Pd(PPh₃)₄ (0.051 g, 0.04 mmol) was added. The reaction was heated to 115° C. for 3.5 h under microwave conditions. After concentration in vacuo, the residue was purified by Gilson HPLC (MeCN/0.1% TFA in water). To the purified product was added MeOH (1 mL) and HCl in Et₂O (2 M, 0.5 mL) which following concentration in vacuo afforded the HCl salt of the title compound (0.014 g, 8% yield). ¹H NMR (DMSO-d₆) [symbol] 5.29 (s, 2H), 7.06 (br s, 1H), 7.18 (d, 2H), 7.26 (br s, 1H), 7.39 (d, 1H), 7.55 (d, 1H), 7.62 (d, 1H), 7.85 (m, 2H), 8.00 (d, 2H), 8.14 (d, 1H), 8.60 (d, 1H), 10.01 (s, 1H), 12.66 (br s, 1H). MS (M+H⁺)=405.

The following Example 106 was prepared in a similar fashion to Example 105 utilizing commercially available starting materials:

| | | | MS | |
|---|---|---|---|---|
| Ex. | Name | MW | (M + H⁺) | ¹H NMR ( [symbol] ppm) |
| 106 | N-[5-(1H- | 454.92 | 455 | 5.35 (s, 2 H), 7.23 (m, 2 H), 7.50 (m, 3 H), |
| | benzimidazol-2-yl)-2- | | | 7.64 (d, 1 H), 7.80 (dd, 2 H), 7.94 (m, 3 H), |
| | chlorophenyl]-4- | | | 8.05 (m, 2 H), 8.16 (m, 1 H), 8.51 (d, 1 H), |
| | (pyridin-2- | | | 8.67 (br s, 1 H), 10.24 (s, 1 H) |
| | ylmethoxy)benzamide | | | |

### Example 107

### N-[5-(1H-imidazol-2-yl)-2-methylphenyl]-4-(pyridin-2-ylethynyl)benzamide

### 107a. Methyl 4-(pyridin-2-ylethynyl)benzoate

In a 200 mL round-bottomed flask was combined methyl 4-bromobenzoate (4.35 g, 20.23 mmol), 2-ethynylpyridine (2.086 g, 20.23 mmol), and cuprous iodide (0.193 g, 1.01 mmol) in DMF (28.9 mL) to give a brown suspension. TEA (30 mL, 215.24 mmol) was added. Nitrogen was bubbled in for 15 min before bis(triphenylphosphine)palladium chloride (0.426 g, 0.61 mmol) was added. The reaction was heated to 50° C. overnight. After concentration under reduced pressure, the residue was diluted with water (30 mL) and EtOAc (30 mL). After filtration, the aqueous layer was extracted with EtOAc (2×15 mL), the combined organic layers were dried (Na₂SO₄) and concentrated to give the crude product that was purified by ISCO MPLC (0-50% EtOAc/hexane) to give the title compound. ¹H NMR (DMSO-d₆) [symbol] 3.88 (s, 3H), 7.46 (m, 1H), 7.71 (d, 1H), 7.76 (m, 2H), 7.89 (td, 1H), 8.02 (m, 2H), 8.64 (d, 1H).

### 107b. 4-(pyridin-2-ylethynyl)benzoic acid

In a 150 mL round-bottomed flask was added methyl 4-(pyridin-2-ylethynyl)benzoate (1.75 g, 7.38 mmol) and LiOH (0.353 g, 14.75 mmol) in MeOH (24.59 mL) to give a white suspension. Water (1 mL) was added and the reaction was heated to 60° C. for 2 h. After concentration in vacuo, the residue was diluted with water (20 mL). Aqueous HCl (1N) was slowly added to the solution to adjust pH to 3. The white precipitate was collected by filtration to give the title compound. MS (M+H⁺)=224.

### 107c. N-[5-(1H-imidazol-2-yl)-2-methylphenyl]-4-(pyridin-2-ylethynyl)benzamide

In a 100 mL round-bottomed flask was placed 4-(pyridin-2-ylethynyl)benzoic acid (0.7 g, 3.14 mmol) and SOCl₂ (0.229 mL, 3.14 mmol) to give a white suspension. The mixture was heated to 60° C. for 3 h. Concentration under reduced pressure gave a residue that was further dried in vacuo at 50° C. for 2 h. To the residue was added pyridine (10 mL) and DCM (10 mL), and 5-(1H-imidazol-2-yl)-2-methylaniline (0.543 g, 3.14 mmol). The reaction was heated to 50° C. and stirred for 2 h. After concentration in vacuo, the residue was diluted with water (20 mL) and DCM (30 mL). The aqueous layer was extracted with DCM (2×10 mL) and the combined organic phases was concentrated. The crude product was purified by ISCO MPLC (0-7% MeOH/DCM) to give the title compound (0.25 g, 21% yield). ¹H NMR (DMSO-d₆) δ 2.27 (s, 3H), 7.14 (s, 2H), 7.37 (d, 1H), 7.46 (ddd, 4.80, 1H), 7.76 (m, 4H), 7.91 (m, 2H), 8.08 (d, 2H), 8.65 (d, 1H), 10.14 (s, 1H), 12.61 (br s, 1H). MS (M+H⁺)=379.

### Example 108

### N-[5-(1H-imidazol-2-yl)-2-methylphenyl]-4-(2-pyridin-2-ylethyl)benzamide

In a 50 mL round-bottomed flask was dissolved N-(5-(1H-imidazol-2-yl)-2-methylphenyl)-4-(pyridin-2-ylethynyl)benzamide (0.06 g, 0.16 mmol) in MeOH (5.0 mL) to give a colorless solution. Nitrogen was bubbled in for 15 min before Pd/C (10%, 0.05 g) was added. To the flask was fitted with a hydrogen balloon and the reaction was kept stirring at RT overnight. After filtration through a short pad of silica gel, the crude product was purified by Gilson HPLC (5-75% MeCN/0.1% TFA in water). To the pure product was added HCl in Et₂O (0.5 mL). Concentration in vacuo gave the title compound as its HCl salt (0.048 g, 79% yield). ¹H NMR (DMSO-d₆) [symbol] 2.34 (s, 3H), 3.18 (m, 2H), 3.35 (d, 2H), 7.45 (d, 2H), 7.57 (d, 1H), 7.80 (m, 3H), 7.85 (d, 1H), 7.95 (m, 3H), 8.14 (s, 1H), 8.36 (t, 1H), 8.78 (d, 1H), 10.13 (s, 1H), 14.91 (br s, 1H). MS (M+H⁺)=383.

### Example 109

### N-[5-(1H-imidazol-2-yl)-2-methylphenyl]-4-[(E)-2-pyridin-2-ylethenyl]benzamide

In a 50 mL round-bottomed flask was added N-(5-(1H-imidazol-2-yl)-2-methylphenyl)-4-(pyridin-2-ylethynyl)benzamide (0.07 g, 0.18 mmol) in THF (9.25 mL) to give a brown suspension. DIBAL-H (0.617 mL, 0.92 mmol) was added, and the solution became clear. The reaction was heated to 60° C. for 3 h. After cooling down to RT, to the reaction water (10 mL) and EtOAc (10 mL) were added. The aqueous layer was extracted with EtOAc (2×5 mL), dried (Na₂SO₄). The combined organic phases were concentrated in vacuo to give the crude product. The crude product was purified by Gilson HPLC (MeCN/0.1% TFA in water). The collected fractions were concentrated to give the title compound (0.012 g, 17% yield). ¹H NMR (DMSO-d₆) [symbol] 2.37 (s, 3H), 7.55 (m, 3H), 7.82 (s, 2H), 7.91 (m, 5H), 8.10 (m, 3H), 8.15 (s, 1H), 8.70 (d, 1H), 10.24 (s, 1H), 14.86 (br s, 1H). MS (M+H⁺)=381.

### Example 110

### N-[5-(1H-imidazol-2-yl)-2-methylphenyl]-4-(1-pyridin-2-ylethoxy)benzamide

### 110a. Methyl 4-(1-(pyridin-2-yl)ethoxy)benzoate

In a 100 mL round-bottomed flask was dissolved 1-(pyridin-2-yl)ethanol (0.90 g, 7.31 mmol), TEA (1.528 mL, 10.96 mmol), and DMAP (0.045 g, 0.37 mmol) in DCM (20 mL) to give a colorless solution. Methanesulfonyl chloride (0.598 mL, 7.67 mmol) was added and the reaction was stirred at RT overnight. The solution was washed with water (20 mL) and the organic layer was dried (Na₂SO₄), filtered, and concentrated in vacuo. The crude product was purified by ISCO MPLC (0-5% MeOH in DCM) to give 1-(pyridin-2-yl)ethyl methanesulfonate. To the product was added methyl 4-hydroxybenzoate (0.867 g, 5.70 mmol) and K₂CO₃ (2.148 g, 15.55 mmol) in MeCN (50 mL) to give a white suspension. The reaction was heated to 85° C. and stirred for 2 h. After concentration in vacuo, the residue was diluted with water (20 mL) and DCM (30 mL). The aqueous layer was extracted with DCM (2×15 mL) and the combined organic layers were dried (Na₂SO₄), and concentrated in vacuo to give the crude product which was purified by ISCO MPLC (0-5% MeOH/DCM) to afford the title compound. ¹H NMR (DMSO-d₆) δ 1.62 (d, 3H), 3.78 (s, 3H), 5.59 (q, 1H), 7.01 (d, 2H), 7.31 (dd, 1H), 7.43 (d, 1H), 7.81 (m, 3H), 8.57 (d, 1H).

### 110b. 4-(1-(pyridin-2-yl)ethoxy)benzoic acid

In a 100 mL round-bottomed flask was combined methyl 4-(1-(pyridin-2-yl)ethoxy)benzoate (1.3 g, 5.05 mmol) and LiOH (0.484 g, 20.21 mmol) in MeOH (30 mL) to give a white suspension. The solution was heated to 60° C. for 5 h. After removal of the solvents in vacuo water (10 mL) was added. The solution became clear, and the pH was adjusted to 4 by the slow addition of 3N HCl. The precipitate was collected by filtration to give the title compound. ¹H NMR (DMSO-d₆) [symbol] 1.61 (d, 3H), 5.57 (q, 1H), 6.98 (d, 2H), 7.31 (dd, 1H), 7.42 (d, 1H), 7.79 (m, 3H), 8.56 (d, 1H), 12.61 (br s, 1H).

### 110c. N-[5-(1H-imidazol-2-yl)-2-methylphenyl]-4-(1-pyridin-2-ylethoxy)benzamide

In a 100 mL round-bottomed flask was dissolved 4-(1-(pyridin-2-yl)ethoxy)benzoic acid (0.15 g, 0.62 mmol) and SOCl₂ (0.900 mL, 12.33 mmol) to give a colorless solution. The reaction was stirred at RT for 1 h. After concentration in vacuo, the solid residue was further dried in a vacuum oven for 2 h to give 4-(1-pyridin-2-ylethoxy)benzoyl chloride. To the acid chloride was added DCM (2 mL), pyridine (5 mL), and 5-(1H-imidazol-2-yl)-2-methylaniline (0.107 g, 0.62 mmol). The reaction was heated to 50° C. and stirred for 2 h. After concentration in vacuo, the crude product was purified by Gilson HPLC (MeCN/10 mM NH₄OAc in water) to give the title compound (0.032 g, 13% yield). ¹H NMR (DMSO-d₆) [symbol] 1.63 (d, 3H), 2.21 (s, 3H), 5.61 (q, 1H), 7.03 (d, 2H), 7.10 (br s, 2H), 7.32 (m, 2H), 7.45 (d, 1H), 7.71 (dd, 1H), 7.80 (td, 1H), 7.90 (m, 3H), 8.58 (d, 1H), 9.78 (s, 1H), 12.43 (br s, 1H). MS (M+H⁺)=399.

### Example 111

### N-[5-(1H-imidazol-2-yl)-2-methylphenyl]-6-(pyridin-2-ylmethoxy)pyridine-3-carboxamide

### 111a. Methyl 6-(pyridin-2-ylmethoxy)nicotinate

In a 200 mL round-bottomed flask was added methyl 6-hydroxynicotinate (1.211 g, 7.91 mmol), 2-(bromomethyl)pyridine hydrobromide (2.0 g, 7.91 mmol), and K₂CO₃ (4.37 g, 31.63 mmol) in MeCN (30 mL) to give a white suspension. The reaction was stirred at RT overnight. The solvent was removed under reduced pressure, and to the residue was added water (20 mL) and DCM (30 mL). The aqueous layer was extracted with DCM (2×10 mL) and the combined organic phases were concentrated to give the crude product, which was purified by ISCO MPLC (30-100% EtOAc/hexane) to give the title compound (Y1.8 g, 95% yield). ¹H NMR (DMSO-d₆) [symbol] 3.80 (s, 3H), 5.30 (s, 2H), 6.44 (d, 1H), 7.29 (dd, 1H), 7.34 (d, 1H), 7.78 (td, 1H), 7.84 (dd, 1H), 8.48 (d, 1H), 8.66 (d, 1H). MS (M+H⁺)=245.

### 111b. 6-(pyridin-2-ylmethoxy)nicotinic acid

In a 500 mL round-bottomed flask was combined methyl 6-(pyridin-2-ylmethoxy)nicotinate (1.36 g, 5.57 mmol) and LiOH (0.667 g, 27.84 mmol) in EtOH (25 mL) to give a colorless suspension. The reaction was stirred at RT overnight. After concentration in vacuo, the white solid residue was dissolved in water (15 mL) and the pH was adjusted to 6 by the careful addition of 1N HCl. After stirring at RT for 15 min, filtration afforded the title compound as a white solid. MS (M+H⁺)=231.

### 111c. N-[5-(1H-imidazol-2-yl)-2-methylphenyl]-6-(pyridin-2-ylmethoxy)pyridine-3-carboxamide

In a 100 mL round-bottomed flask was combined 6-(pyridin-2-ylmethoxy)nicotinic acid (0.092 g, 0.40 mmol) and SOCl₂ (0.674 mL, 9.24 mmol) in DCM (2 mL) to give a white suspension. The reaction was heated to 50° C. for 2 h, and the reaction became a clear solution. Concentration under reduced pressure gave a solid residue, which was further dried in a vacuum oven for 2 h to give 6-(pyridin-2-ylmethoxy)pyridine-3-carbonyl chloride. To the residue was added DCM (2 mL), pyridine (2 mL), and 5-(1H-imidazol-2-yl)-2-methylaniline (0.08 g, 0.46 mmol). The reaction was heated to 50° C. for 2 h. After concentration under reduced pressure, the crude product was purified by Gilson HPLC (MeCN/0.1% TFA in water) to give a residue that was diluted with MeOH (1 mL) and HCl in Et₂O (0.5 M, 1 mL). The solution was concentrated under reduced pressure to give the title compound (0.061 g, 34% yield) as an HCl salt. ¹H NMR (DMSO-d₆) [symbol] 2.34 (s, 3H), 5.35 (s, 2H), 6.53 (d, 1H), 7.44 (d, 2H), 7.57 (d, 1H), 7.80 (s, 2H), 7.91 (m, 2H), 8.10 (s, 2H), 8.58 (br s, 1H), 8.76 (d, 1H), 10.07 (s, 1H), 14.89 (br s, 2H). MS (M+H⁺)=386.

### Example 112

### N-[5-(1H-imidazol-2-yl)-2-methylphenyl]-5-(pyridin-2-ylmethoxy)pyridine-2-carboxamide

### 112a. Methyl 5-(pyridin-2-ylmethoxy)picolinate

In a 200 mL round-bottomed flask was added methyl 5-hydroxypicolinate (2.092 g, 13.66 mmol), 2-(bromomethyl)pyridine hydrobromide (3.46 g, 13.66 mmol), and K₂CO₃ (1.888 g, 13.66 mmol) in MeCN (110 mL) to give a suspension. The reaction was heated to 80° C. for 2 h. After Concentration under reduced pressure, the residue was diluted with water (20 mL) and DCM (50 mL). The aqueous layer was extracted with DCM (2×30 mL), and the combined organic layers were dried (Na₂SO₄) to give the crude product that was purified by ISCO MPLC (10% MeOH/DCM) to give the title compound. ¹H NMR (DMSO-d₆) [symbol] 3.85 (s, 3H), 5.36 (s, 2H), 7.38 (dd, 1H), 7.60 (m, 2H), 7.87 (td, 1H), 8.05 (d, 1H), 8.49 (d, 1H), 8.60 (d, 1H).

### 112b. 5-(pyridin-2-ylmethoxy)picolinic acid

In a 200 mL round-bottomed flask was combined methyl 5-(pyridin-2-ylmethoxy)picolinate (1.66 g, 6.80 mmol) and LiOH (0.651 g, 27.19 mmol) in MeOH (40 mL) to give a colorless suspension. The reaction was heated to 60° C. and was stirred overnight. After Concentration under reduced pressure, the solid was diluted with water (15 mL). To the solution was slowly added concentrated HCl solution adjusting the pH to 5. The precipitate was collected by filtration to give the title compound. ¹H NMR (DMSO-d₆) [symbol] 5.36 (s, 2H), 7.39 (dd, 1H), 7.59 (m, 2H), 7.87 (td, 1H), 8.03 (d, 1H), 8.47 (d, 1H), 8.60 (d, 1 H).

### 112c. N-[5-(1H-imidazol-2-yl)-2-methylphenyl]-5-(pyridin-2-ylmethoxy)pyridine-2-carboxamide

In a 100 mL round-bottomed flask was placed 5-(pyridin-2-ylmethoxy)picolinic acid (0.16 g, 0.69 mmol) and SOCl₂ (1.015 mL, 13.90 mmol) to give a white suspension. The mixture was heated to 80° C. for 2 h. Concentration under reduced pressure gave 5-(pyridin-2-ylmethoxy)pyridine-2-carbonyl chloride that was further dried in a vacuum oven for 2 h at 50° C. To the residue was added 5-(1H-imidazol-2-yl)-2-methylaniline (0.12 g, 0.69 mmol). The reaction mixture was dissolved in pyridine (2 mL) and DCM (2 mL) and the solution was heated to 50° C. and stirred for 1 h. After concentration in vacuo, the crude product was purified by ISCO MPLC (20% MeOH/DCM) to give the title compound (0.041 g, 15% yield). ¹H NMR (DMSO-d₆) [symbol] 2.32 (s, 3H), 5.40 (s, 2H), 7.22 (br s, 2H), 7.39 (m, 2H), 7.60 (d, 1H), 7.75 (m, 2H), 7.89 (td, 1H), 8.15 (d, 1H), 8.40 (s, 1H), 8.53 (d, 1H), 8.62 (d, 1H), 10.18 (s, 1H), 12.01 (br s, 1H). MS (M+H⁺)=386.

### Example 113

### N-(2,4-dimethyl-5-(1-methyl-1H-imidazol-4-yl)phenyl)-5-(pyridin-2-ylmethoxy)picolinamide

### 113a. 2,4-dimethyl-5-(1-methyl-1H-imidazol-4-yl)aniline

In a 100 mL round-bottomed flask was added 4,4,4′,4′,5,5,5′,5′-octamethyl-2,2′-bi(1,3,2-dioxaborolane) (1.0 g, 3.94 mmol), 5-bromo-2,4-dimethylaniline (0.525 g, 2.63 mmol), and potassium acetate (0.773 g, 7.88 mmol) in dioxane (70 mL) to give a yellow suspension. Nitrogen was bubbled in for 20 min before Pd(PPh₃)₄ (0.455 g, 0.39 mmol) was added. The reaction was heated to 110° C. for 15 h. After it was cooled down to RT, the mixture was concentrated under reduced pressure. The crude product was purified by ISCO MPLC (0-5% MeOH/DCM) to give 2,4-dimethyl-5-(4,4,5,5-tetramethyl-1,3,2-dioxaborolan-2-yl)aniline. In a 100 mL round-bottomed flask was combined 2,4-dimethyl-5-(4,4,5,5-tetramethyl-1,3,2-dioxaborolan-2-yl)aniline, 4-bromo-1-methyl-1H-imidazole (0.81 g, 5.03 mmol), and KOAc (0.823 g, 8.39 mmol) in dioxane (15 mL) to give a yellow suspension. The reaction mixture was diluted with water (5.0 mL) and nitrogen was bubbled in for 20 min before Pd(PPh₃)₄ (0.388 g, 0.34 mmol) was added. The reaction was heated to 110° C. for 50 h. After cooling to RT, the reaction mixture was concentrated in vacuo and the residue was pre-absorbed on silica gel and purified by ISCO MPLC (5-20% MeOH/DCM) to give the title compound (0.14 g, 21% yield). ¹H NMR (DMSO-d₆) [symbol] ppm 2.02 (s, 3H), 2.23 (s, 3H), 3.67 (s, 3H), 4.56 (s, 2H), 6.73 (s, 1H), 7.11 (s, 1H), 7.18 (s, 1H), 7.58 (s, 1H). MS (M+H⁺)=202.

### 113b. N-(2,4-dimethyl-5-(1-methyl-1H-imidazol-4-yl)phenyl)-5-(pyridin-2-ylmethoxy)picolinamide

Prepared in a similar fashion to Example 112, step c using 2,4-dimethyl-5-(1-methyl-1H-imidazol-4-yl)aniline to give the title compound. ¹H NMR (DMSO-d₆) δ ppm 2.25 (s, 3H), 2.40 (s, 3H), 3.71 (s, 3H), 5.39 (s, 2H), 7.09 (s, 1H), 7.38 (m, 2H), 7.59 (d, 1H), 7.70 (m, 2H), 7.88 (m, 1H), 8.11 (d, 1H), 8.20 (s, 1H), 8.50 (d, 1H), 8.61 (d, 1H), 10.00 (s, 1H). MS (M+H⁺)=414.

### Example 114

### N-(2-methyl-5-(1H-1,2,3-triazol-4-yl)phenyl)-4-(pyridin-2-ylmethoxy)benzamide

### 114a. 5-ethynyl-2-methylaniline

In a 500-mL round-bottomed flask was placed 5-bromo-2-methylaniline (9 g, 0.048 mol), copper(I) iodide (0.92 g, 0.005 mol), and TEA (50 mL) in DMF (50 mL). Nitrogen was bubbled in for 5 min. To the mixture was added Pd(PPh₃)₄ (5.6 g, 0.005 mol). The reaction was stirred at 80 □C overnight. Concentration in vacuo removed solvents, and to the residue was added THF (100 mL). After filtration, the filtrate was concentrated to give the crude product, which was purified by ISCO MPLC (EtOAc and hexane) to afford the title compound. ¹H NMR (CDCl₃) δ 2.18 (s, 3H), 3.03 (s, 1H), 3.63 (s, 2H), 6.82 (s, 1H), 6.90 (d, 1H), 7.00 (d, 1H).

### 114b. N-(5-ethynyl-2-methylphenyl)-4-(pyridin-2-ylmethoxy)benzamide

In a 100-mL round-bottomed flask was placed 4-(pyridin-2-ylmethoxy)benzoic acid (2.0 g, 8.7 mmol) (prepared in Example 1, step a-b), 5-ethynyl-2-methylaniline (1.1 g, 8.7 mmol), and DIPEA (3.1 mL, 17.5 mmol) in DMF (20 mL). To the mixture was added HATU (3.32 g, 8.7 mmol) and the reaction was stirred at RT overnight. The reaction mixture was poured into water (100 mL), and the suspension was stirred at RT for 30 min. Filtration afforded the crude product as a solid, which was suspended in NaOH (10N, 30 mL) and MeOH (30 mL). The suspension was stirred at RT overnight. After filtration and concentration of the filtrate, the resultant solid residue was washed with water (2×20 mL) and dried in a vacuum oven to give the title compound. ¹H NMR (DMSO-d₆) δ 2.22 (s, 3H), 4.10 (s, 1H), 5.26 (s, 2H), 7.12 (d, 2H), 7.25 (m, 2H), 7.35 (m, 1H), 7.45 (s, 1H), 7.52 (d, 1H), 7.83 (m, 1H), 7.94 (d, 2H), 8.57 (m, 1H). MS (M+H⁺)=344.

### 114c. N-(2-methyl-5-(1H-1,2,3-triazol-4-yl)phenyl)-4-(pyridin-2-ylmethoxy)benzamide

To a solution of N-(5-ethynyl-2-methylphenyl)-4-(pyridin-2-ylmethoxy)benzamide (0.209 g, 0.61 mmol) and copper(I) iodide (5.81 mg, 0.03 mmol) in DMF (1.099 mL) and MeOH (0.122 mL) was added trimethylsilyl azide (0.122 mL, 0.92 mmol). The solution was heated in a microwave at 100° C. for 12 h. After cooling, the reaction was added to sat.NaHCO₃ (1 mL) and water (10 mL). The precipitate was collected by filtration and washed with water. The solid was purified by ISCO MPLC (DCM to 91:8:1 DCM: MeOH: NH₄OH), then by reverse phase HPLC (10-60% MeCN/10 mM NH₄OAc in water) to yield the title compound (0.051 g, 21.72%). ¹H NMR (DMSO-d₆) [symbol] 15.01 (br s, 1H), 9.84 (s, 1H), 8.59 (d, 1H), 8.27 (br s, 1H), 7.98 (d, 2H), 7.85 (m, 2H), 7.65 (d, 1H), 7.54 (d, 1H), 7.36 (m, 2H), 7.16 (d, 2H), 5.28 (s, 2H), 2.24 (s, 3H). MS (M+H⁺)=386.

### Example 115

### 4-(2-cyano-5-(4-methylpiperazin-1-yl)benzyloxy)-N-(2-methyl-5-(1-methyl-1H-imidazol-2-yl)phenyl)benzamide

A mixture of 4-(2-cyano-5-fluorobenzyloxy)-N-(2-methyl-5-(1-methyl-1H-imidazol-2-yl)phenyl)benzamide (40 mg, 0.09 mmol), 1-methylpiperazine (45.5 mg, 0.45 mmol), K₂CO₃ (62.8 mg, 0.45 mmol) in DMF (3 mL) was stirred at 100° C. for 3 h and after cooling the reaction was filtered and washed with EtOAc. The filtrate was concentrated in vacuo, and the residue was purified with Gilson HPLC (5-50% MeCN/0.1% TFA in water) to yield the title compound as a white solid (25.0 mg, 49.4%). ¹H NMR (DMSO-d₆) [symbol] 14.79 (br s, 1H), 11.30 (br s, 1H), 10.02 (s, 1H), 7.98 (m, 2H), 7.75-7.87 (m, 2H), 7.73 (d, 1H), 7.67 (d, 1H), 7.42-7.59 (m, 2H), 7.31 (d, 1H), 7.14 (m, 2H), 7.06 (dd, 1H), 5.16 (s, 2H), 4.03 (br s, 2H), 3.83 (s, 3H), 3.34-3.49 (m, 4H), 3.04 (br s, 2H), 2.72 (s, 3H), 2.31 (s, 3H). MS (M+H⁺)=521.

The following Example 1116 was prepared in a similar fashion to Example 115 utilizing commercially available starting materials:

| | | | MS | |
|---|---|---|---|---|
| Ex. | Name | MW | (M + H⁺) | ¹H NMR (δ ppm) |
| 116 | 4-[(2-cyano-5-{[2- | 522.65 | 523 | 11.05 (br s, 1 H), 10.00 (br s, 1 H), 7.97 (m, |
| | (dimethylamino)ethyl](methyl)amino}benzyl)oxy]- | | | 2 H), 7.79 (m, 2 H), 7.73 (br s, 1 H), 7.58 (d, |
| | N-[2-methyl-5-(1- | | | 1 H), 7.52 (br s, 2 H), 7.15 (br s, 3 H), |
| | methyl-1H-imidazol-2- | | | 6.84 (br s, 1 H), 5.18 (br s, 2 H), 3.83 (br s, 5 H), |
| | yl)phenyl]benzamide | | | 3.13 (br s, 2 H), 2.97 (br s, 3 H), 2.72 (br s, |
| | | | | 6 H), 2.31 (br s, 3 H) |

### Example 117

### N-(5-(1H-imidazol-2-yl)-2-methylphenyl)-4-(pyridin-2-ylmethylamino)benzamide

### 117a. 5-(1H-imidazol-2-yl)-2-methylaniline

A mixture of 2-iodo-1H-imidazole (4.7 g, 24.23 mmol), 3-amino-4-methylphenylboronic acid hydrochloride (4.6 g, 24.54 mmol), KOAc (7.13 g, 72.69 mmol) and Pd(PPh₃)₄ (1.400 g, 1.21 mmol) in dioxane (30 mL) and water (7.50 mL) was subjected to microwave for 0.5 h at 150° C. The mixture was concentrated in vacuo and the residue was purified with ISCO MPLC (0-6% MeOH/DCM) to yield a brown solid that was repurified with Gilson HPLC (1-40% MeCN/0.1% TFA in water) to yield the title compound as a solid (1.800 g, 42.9%). ¹H NMR (DMSO-d₆) [symbol] 7.85 (s, 2H), 7.14-7.35 (m, 3H), 2.26 (s, 3H). MS (M+H⁺)=174.

### 117b. tert-butyl-4-(5-(1H-imidazol-2-yl)-2-methylphenylcarbamoyl)phenylcarbamate

A mixture of 4-(tert-butoxycarbonylamino)benzoic acid (548 mg, 2.31 mmol), 5-(1H-imidazol-2-yl)-2-methylaniline (400 mg, 2.31 mmol), HATU (966 mg, 2.54 mmol) and DIPEA (1.613 mL, 9.24 mmol) in DMF (6 mL) was stirred at RT for 2 h. The temperature was increased to 50° C. and stirred overnight. After concentration in vacuo, the residue was purified with ISCO MPLC (60-100% EtOAc/hexane to 40% MeOH/EtOAc) to afford the title compound (390 mg, 43.0%). MS (M+H⁺)=393.

### 117c. N-(5-(1H-imidazol-2-yl)-2-methylphenyl)-4-aminobenzamide

A mixture of tert-butyl 4-(5-(1H-imidazol-2-yl)-2-methylphenylcarbamoyl)phenylcarbamate (390 mg, 0.99 mmol) in 4M HCl in dioxane (5 mL, 143.99 mmol) was stirred at RT for 2 h. The solid was collected by filtration, washed with Et₂O, and dried to yield the title compound (282 mg, 86%). ¹H NMR (DMSO-d₆) [symbol] 9.89 (s, 1H), 8.15 (d, 1H), 7.99 (dd, 1H), 7.89 (m, 2H), 7.78 (s, 2H), 7.54 (d, 1H), 6.93 (m, 2H), 2.34 (s, 3H). MS (M+H⁺)=293.

### 117d. N-(5-(1H-imidazol-2-yl)-2-methylphenyl)-4-(pyridin-2-ylmethylamino)benzamide

To a mixture of picolinaldehyde (53.6 mg, 0.5 mmol), N-(5-(1H-imidazol-2-yl)-2-methylphenyl)-4-aminobenzamide hydrochloride (140 mg, 0.43 mmol) in DCM (10 mL) was added sodium triacetoxyborohydride (316 mg, 1.49 mmol) at RT. The reaction mixture was stirred at RT overnight. The mixture was concentrated in vacuo and the residue was purified with Gilson HPLC (MeCN/0.1% TFA in water) to yield the title compound (100 mg, 55.9%). ¹H NMR (DMSO-d₆) [symbol] 9.80 (s, 1H), 8.80 (d, 1H), 8.47 (t, 1H), 8.09 (s, 1H), 8.00 (d, 1H), 7.93 (d, 1H), 7.87 (t, 1H), 7.79 (d, 2H), 7.62-7.72 (m, 2H), 7.43 (d, 1H), 6.70 (d, 2H), 4.81 (s, 2H), 2.22 (s, 3H). MS (M+H⁺)=384.

The following Example 118 were prepared in a similar fashion to Example 117 utilizing commercially available starting materials:

| | | | MS | |
|---|---|---|---|---|
| Ex. | Name | MW | (M + H⁺) | ¹H NMR (δ ppm) |
| 118 | N-[5-(1H-imidazol-2-yl)-2- | 398 | 397.48 | 14.88 (br s, 2 H), 9.64 (s, 1 H), 8.68 (d, 1 H), |
| | methylphenyl]-4-[(1- | | | 8.22 (br s, 1 H), 8.05 (d, 1 H), 7.87 (dd, 1 H), |
| | pyridin-2- | | | 7.78 (br s, 1 H), 7.68-7.74 (m, 4 H), 7.64 (d, |
| | ylethyl)amino]benzamide | | | 1 H), 7.45 (d, 1 H), 6.60 (d, 2 H), 4.95 (d, 1 H), |
| | | | | 2.22 (s, 3 H), 1.53 (d, 3 H) |

### Example 119

### N-(5-(1H-imidazol-2-yl)-2-methylphenyl)-4-((6-bromopyridin-2-yl)methoxy)benzamide

### 119a. (6-bromopyridin-2-yl)methyl 4-methylbenzenesulfonate

To a mixture of (6-bromopyridin-2-yl)methanol (635 mg, 3.38 mmol) and 4-methylbenzene-1-sulfonyl chloride (708 mg, 3.71 mmol) in anhydrous DCM (5 mL) was added TEA (0.941 mL, 6.75 mmol) and DMAP (5 mg, 0.04 mmol). The mixture was stirred at RT for 30 min before sat. NH₄Cl was added to the mixture. After extraction with DCM (3 x), the combined organic layers were dried over anhydrous Na₂SO₄, filtered, and concentrated in vacuo to yield the title compound as a light brown oil (1156 mg, 100%). MS (M+H⁺)=343.

### 119b. N-(5-(1H-imidazol-2-yl)-2-methylphenyl)-4-((6-bromopyridin-2-yl)methoxy)benzamide

A mixture of (6-bromopyridin-2-yl)methyl 4-methylbenzenesulfonate (1.158 g, 3.38 mmol), N-(5-(1H-imidazol-2-yl)-2-methylphenyl)-4-hydroxybenzamide (350 mg, 1.19 mmol) and K₂CO₃ (0.989 g, 7.16 mmol) in MeCN (20 mL) was stirred at 80° C. overnight and then filtered, washed with DCM and MeOH, and concentrated in vacuo to give a residue which was purified with ISCO MPLC (40-100% EtOAc/hexane) to yield the title compound as a light yellow solid (0.475 g, 86%). ¹H NMR (DMSO-d₆) [symbol] 14.64 (br s, 1H), 9.93 (s, 1H), 8.04 (s, 1H), 7.95 (m, 2H), 7.72-7.88 (m, 2H), 7.69 (s, 2H), 7.37-7.62 (m, 3H), 7.13 (m, 2H), 5.22 (s, 2H), 2.27 (s, 3H). MS (M+H⁺)=464.

The following Examples 120-121 were prepared in a similar fashion to Example 119 utilizing commercially available starting materials:

| | | | MS | |
|---|---|---|---|---|
| Ex. | Name | MW | (M + H⁺) | ¹H NMR (δ ppm) |
| 120 | 4-[(3-bromopyridin-2- | 463.33 | 465 | 10.05 (s, 1 H), 8.61 (dd, 1 H), 8.12-8.25 (m, |
| | yl)methoxy]-N-[5-(1H- | | | 2 H), 7.90-8.07 (m, 3 H), 7.79 (s, 2 H), |
| | imidazol-2-yl)-2- | | | 7.56 (d, 1 H), 7.42 (dd, 1 H), 7.19 (d, 2 H), 5.36 (s, |
| | methylphenyl]benzamide | | | 2 H), 2.35 (s, 3 H) |
| 121 | 4-[(3-bromopyridin-2- | 633.34 | 634 | 15.14 (br s, 1 H), 9.83 (s, 1 H), 8.53 (dd, H), |
| | yl)methoxy]-N-(5-{1- | | | 8.26-8.44 (m, 1 H), 8.10 (ddd, 8.08, 2 H), |
| | [(3-bromopyridin-2- | | | 7.87 (d, 2 H), 7.81 (s, 2 H), 7.66 (d, 1 H), |
| | yl)methyl]-1H- | | | 7.37-7.48 (m, 1 H), 7.31-7.37 (m, 3 H), |
| | imidazol-2-yl}-2- | | | 7.18-7.31 (m, 3 H), 7.01-7.17 (m, 3 H), 5.64 (s, 2 |
| | methylphenyl)benzamide | | | H), 5.28 (s, 2 H), 2.26 (s, 3 H) |

### Example 122

### N-(5-(1H-imidazol-2-yl)-2-methylphenyl)-4-(6-(2-hydroxyethoxy)pyridin-2-yl)methoxy)benzamide

A microwave vial was charged with ethane-1,2-diol (0.512 mL, 9.17 mmol) and NaH (60% in mineral oil) (66.0 mg, 2.75 mmol). The mixture was stirred at RT for 1 h before N-(5-(1H-imidazol-2-yl)-2-methylphenyl)-4-((6-bromopyridin-2-yl)methoxy)benzamide (85 mg, 0.18 mmol) in DMF (1 mL) was added. The mixture was subjected to microwave conditions for 30 min at 150° C., then concentrated in vacuo. The residue was purified with Gilson HPLC (2-65% MeCN/0.1% TFA in water). The collected fractions were concentrated and then repurified with Gilson HPLC (5-70% MeCN/10 mM NH₄OAc in water) to yield the title compound as a white solid (5.0 mg, 6.13%). ¹H NMR (DMSO-d₆) [symbol] 12.45 (br s, 1H), 9.84 (s, 1H), 7.99 (d, 2H), 7.90 (d, 1H), 7.66-7.82 (m, 2H), 7.33 (d, 1H), 7.12-7.25 (m, 3H), 7.10 (d, 1H), 6.99 (s, 1H), 6.77 (d, 1H), 5.18 (s, 2H), 4.84 (t, 1H), 4.25-4.31 (m, 2H), 3.71 (q, 2H), 2.24 (s, 3H). MS (M+H⁺)=445.

### Example 123

### N-(5-(1H-imidazol-2-yl)-2-methylphenyl)-4-((6-methoxypyridin-2-yl)methoxy)benzamide

A mixture of N-(5-(1H-imidazol-2-yl)-2-methylphenyl)-4-((6-bromopyridin-2-yl)methoxy)benzamide (90 mg, 0.19 mmol) in 0.5 M sodium methoxide in MeOH (2 mL) was subjected to microwave conditions for 30 min at 150° C. Concentration under reduced pressure gave a residue which was purified with Gilson HPLC (2-85% MeCN/0.1% TFA in water) to yield the title compound as a white solid (12.0 mg, 13.70%). ¹H NMR (DMSO-d₆) [symbol] 9.95 (s, 1H), 8.08 (d, 1H), 8.00 (m, 2H), 7.64-7.85 (m, 5H), 7.56 (d, 1H), 7.20 (m, 2H), 7.12 (d, 1H), 6.79 (d, 1H), 5.21 (s, 2H), 3.86 (s, 3H), 2.34 (s, 3H). MS (M+H⁺)=415.

### Example 124

### N-(5-(1H-imidazol-2-yl)-2-methylphenyl)-4-((6-(2-(dimethylamino)ethoxy)pyridin-2-yl)methoxy)benzamide

To a microwave vial was added 2-(dimethylamino)ethanol (0.585 mL, 5.83 mmol) and NaH (60% in mineral oil) (62.2 mg, 1.55 mmol). The mixture was stirred at RT for 1 h before N-(5-(1H-imidazol-2-yl)-2-methylphenyl)-4-((6-bromopyridin-2-yl)methoxy)benzamide (90 mg, 0.19 mmol) in DMF (1 mL) was added to the mixture. Then the mixture was subjected to microwave conditions for 30 min at 150° C. After concentration in vacuo, the residue was purified with Gilson HPLC (1-50% MeCN/0.1% TFA in water). The collected fractions were concentrated and then repurified with Gilson HPLC (2-70% MeCN/10 mM NH₄OAc in water) to yield the title compound as a white solid (30.0 mg, 32.8%). ¹H NMR (DMSO-d₆) [symbol] 9.85 (s, 1H), 8.00 (d, 2H), 7.91 (s, 1H), 7.82 (t, 1H), 7.72 (dd, 1H), 7.34 (d, 1H), 7.04-7.24 (m, 5H), 6.84 (d, 1H), 5.21 (s, 2H), 4.51-4.64 (m, 2H), 3.45 (br s, 2H), 2.82 (s, 6H), 2.19-2.28 (m, 3H). MS (M+H⁺)=472.

### Example 125

### N-(5-(1H-imidazol-2-yl)-2-methylphenyl)-4-((6-(dimethylamino)pyridin-2-yl)methoxy)benzamide

A microwave tube was charged with N-(5-(1H-imidazol-2-yl)-2-methylphenyl)-4-((6-bromopyridin-2-yl)methoxy)benzamide (100 mg, 0.22 mmol), dimethylamine (1 mL, 2.0 mmol) (2M in THF) in 1 mL of DMF. The mixture was subjected to microwave conditions for 30 min at 150° C. The tube was put back in the microwave for 45 min at 150° C. After concentration in vacuo, the residue was purified with Gilson HPLC (2% to 65% MeCN/0.1% TFA in water) to yield the title compound as a white solid (44.0 mg, 43.9%). ¹H NMR (DMSO-d₆) δ 14.87 (br s, 2H), 9.99 (s, 1H), 8.07 (d, 1H), 7.97 (m, 2H), 7.90 (d, 1H), 7.73 (s, 2H), 7.67 (br s, 1H), 7.49 (d, 1H), 7.11 (m, 2H), 6.76 (br s, 2H), 5.18 (br s, 2H), 3.07 (br s, 6H), 2.17-2.32 (m, 3H). MS (M+H⁺)=428.

The following Examples 126-127 were prepared in a similar fashion to Example 125 utilizing commercially available starting materials:

| | | | MS | |
|---|---|---|---|---|
| Ex. | Name | MW | (M + H⁺) | ¹H NMR (δ ppm) |
| 126 | N-[5-(1H-imidazol-2-yl)-2- | 482.59 | 483 | 10.06 (s, 1 H), 8.35 (d, 1 H), 7.94-8.14 (m, |
| | methylphenyl]-4-{[4-(4- | | | 3 H), 7.88 (dd, 1 H), 7.71 (s, 2 H), |
| | methylpiperazin-1- | | | 7.40-7.57 (m, 2 H), 7.07-7.29 (m, 3 H), 5.29 (s, |
| | yl)pyridin-2- | | | 2 H), 4.38 (br s, 2 H), 3.50 (br s, 4 H), |
| | yl]methoxy}benzamide | | | 3.12 (br s, 2 H), 2.73 (s, 3 H), 2.27 (s, 3 H) |
| 127 | 4-{[4- | 427.51 | 428 | 9.77 (s, 1 H), 8.14 (s, 1 H), 8.05 (d, 1 H), |
| | (dimethylamino)pyridin-2- | | | 7.91 (d, 2 H), 7.84 (d, 1 H), 7.66 (dd, 1 H), |
| | yl]methoxy}-N-[5-(1H- | | | 7.26 (d, 1 H), 7.09 (d, 4 H), 6.69 (d, 1 H), |
| | imidazol-2-yl)-2- | | | 6.50 (dd, 1 H), 5.04 (s, 2 H), 2.90 (s, 6 H), |
| | methylphenyl]benzamide | | | 2.17 (s, 3 H) |

### Example 128

### N-(5-(1H-imidazol-2-yl)-2-methylphenyl)-4-((6-(4-methylpiperazin-1-yl)pyridin-2-yl)methoxy)benzamide

A mixture of N-(5-(1H-imidazol-2-yl)-2-methylphenyl)-4-((6-bromopyridin-2-yl)methoxy)benzamide (60 mg, 0.13 mmol) and 1-methylpiperazine (130 mg, 1.29 mmol) in DMF (2.5 mL) was subjected to microwave conditions for 30 min at 160° C. After concentration in vacuo, the residue was purified with Gilson HPLC (2-60% MeCN/0.1% TFA in water) to yield the title compound as a white solid (30.0 mg, 44.6%). ¹H NMR (DMSO-d₆) [symbol] 14.90 (br s, 2H), 11.05 (br s, 1H), 9.98 (s, 1H), 8.08 (d, 1H), 7.86-8.02 (m, 3H), 7.72 (s, 2H), 7.60 (dd, 1H), 7.49 (d, 1H), 7.10 (d, 2H), 6.84 (dd, H), 5.08 (s, 2H), 4.33 (d, 2H), 3.41 (d, 2H), 3.14-3.29 (m, 2H), 2.98 (d, 2H), 2.72 (d, 3H), 2.13-2.32 (m, 3H). MS (M+H⁺)=483.

The following Example 129 was prepared in a similar fashion to Example 128 utilizing commercially available starting materials:

| | | | MS | |
|---|---|---|---|---|
| Ex. | Name | MW | (M + H⁺) | ¹H NMR (δ ppm) |
| 129 | N-[5-(1H-imidazol-2- | 469.54 | 470 | 14.83 (br s, 2 H), 9.95 (s, 1 H), 8.06 (d, 1 |
| | yl)-2-methylphenyl]-4- | | | H), 7.92-8.01 (m, 2 H), 7.88 (dd, 1 H), |
| | [(6-morpholin-4- | | | 7.73 (s, 2 H), 7.55 (d, 1 H), 7.49 (d, 1 H), 7.10 (d, |
| | ylpyridin-2- | | | 2 H), 6.76 (d, 2 H), 5.08 (s, 2 H), |
| | yl)methoxy]benzamide | | | 3.53-3.70 (m, 4 H), 3.32-3.52 (m, 4 H), 2.27 (s, 3 H) |

### Example 130

### N-(5-(1H-imidazol-2-yl)-2-methylphenyl)-4-((5-methoxypyridin-2-yl)methoxy)benzamide

### 130a. 5-methoxypicolinaldehyde

A mixture of 5-fluoropicolinaldehyde (450 mg, 3.60 mmol) and sodium methoxide (291 mg, 5.40 mmol) in MeOH (15 mL) was stirred at 55° C. overnight. The reaction was filtered, washed with MeOH, and the filtrate was concentrated in vacuo to give a residue that was purified with ISCO MPLC (30-45% EtOAc/hexane) to yield the title compound as a colorless oil (326 mg, 66.1%). ¹H NMR (CDCl₃) [symbol] 9.93 (s, 1H), 8.37 (d, 1H), 7.90 (d, 1H), 7.24 (dd, 1H), 3.89 (s, 4H).

### 130b. (5-methoxypyridin-2-yl)-methanol

To a mixture of 5-methoxypicolinaldehyde (326 mgs, 2.36 mmol) in MeOH (10 mL) was added NaBH₄ (71.9 mg, 1.90 mmol) at 0° C. The mixture was stirred at 0° C. for 10 min after concentration in vacuo, the residue was purified with ISCO MPLC (40-80% EtOAc/hexane) to yield the title compound as a colorless oil (298 mg, 90%). ¹H NMR (CDCl₃) [symbol] 8.28 (d, 1H), 7.09-7.27 (m, 2H), 4.73 (s, 2H), 3.89 (s, 3H). MS (M+H⁺)=140.

### 130c. N-(5-(1H-imidazol-2-yl)-2-methylphenyl)-4-((5-methoxypyridin-2-yl)methoxy)benzamide

To a mixture of N-(5-(1H-imidazol-2-yl)-2-methylphenyl)-4-hydroxybenzamide (84 mg, 0.29 mmol), (5-methoxypyridin-2-yl)methanol (40 mg, 0.29 mmol) and PS-triphenylphosphine (298 mg, 0.57 mmol; 1.88 mmol/g) in THF (10 mL) was added (E)-diisopropyl diazene-1,2-dicarboxylate (0.113 mL, 0.57 mmol). The mixture was stirred at RT for 10 min, filtered and washed with DCM. The filtrate was concentrated in vacuo and the residue was purified with Gilson HPLC (2-75% MeCN/0.1% TFA in water) to yield the title compound as a white solid (25.0 mg, 19.29%). ¹H NMR (DMSO-d₆) [symbol] 14.75 (br s, 2H), 9.93 (s, 1H), 8.25 (d, 1H), 8.05 (d, 1H), 7.94 (m, 2H), 7.84 (dd, 1H), 7.73 (s, 2H), 7.44-7.55 (m, 2H), 7.34-7.44 (m, 1H), 7.11 (m, 2H), 5.15 (s, 2H), 3.78 (s, 3H), 2.27 (s, 3H). MS (M+H⁺)=415.

### Example 131

### N-(5-(1H-imidazol-2-yl)-2-methylphenyl)-4-((5-(2-hydroxyethoxy)pyridin-2-yl)methoxy)benzamide

### 131a. 5-(2-hydroxyethoxy)picolinaldehyde

To a mixture of ethane-1,2-diol (3.48 g, 56.0 mmol) in DCE (15 mL) was added NaH (60% in mineral oil) (0.168 g, 4.20 mmol) at RT. The mixture was stirred at RT for 1 h before 5-fluoropicolinaldehyde (0.350 g, 2.8 mmol) was added. The mixture was then refluxed overnight. Water was added to the mixture and extracted with DCM (3×). The combined organic layers were dried over anhydrous Na₂SO₄, filtered, and concentrated in vacuo to yield the title compound as an orange solid (0.294 g, 62.8%). ¹H NMR (CDCl₃) [symbol] 9.93 (s, 1H), 8.40 (d, 1H), 7.91 (d, 1H), 7.27 (dd, 1H), 4.12-4.22 (m, 2H), 3.96-4.02 (m, 2H). MS (M+H⁺)=168.

### 131b. 5-(2-(tent-butyldiphenylsilyloxy)ethoxy)picolinaldehyde

To a mixture of 5-(2-hydroxyethoxy)picolinaldehyde (215 mg, 1.28 mmol) in DMF (5 mL) was added tert-butyldiphenylchlorosilane (0.275 mL, 1.07 mmol) and imidazole (87 mg, 1.28 mmol). The mixture was stirred at RT. A second portion of tert-butyldiphenylchlorosilane (0.275 mL, 1.07 mmol) and imidazole (87 mg, 1.28 mmol) was added to the mixture and stirred for 1 day. The mixture was concentrated in vacuo and the residue was purified with ISCO MPLC (0-30% EtOAc/hexane) to yield the title compound as a light yellow oil (420 mg, 97%). MS (M+H⁺)=406.

### 131c. (5-(2-(tent-butyldiphenylsilyloxy)ethoxy)pyridin-2-yl)methanol

Prepared in a similar fashion to (5-methoxypyridin-2-yl)-methanol (Example 130, step b). ¹H NMR (CDCl₃) [symbol] 8.24 (d, 1H), 7.72 (dd, 4H), 7.36-7.50 (m, 6H), 7.10-7.23 (m, 2H), 4.72 (s, 2H), 4.15 (t, 2H), 4.03 (t, 2H), 1.08 (s, 9H). MS (M+H⁺)=408.

### 131d. N-(5-(1H-imidazol-2-yl)-2-methylphenyl)-4-((5-(2-(tert-butyldiphenylsilyloxy)ethoxy)pyridin-2-yl)methoxy)benzamide

Prepared in a similar fashion to Example 130. MS (M+H⁺)=683.

### 131e. N-(5-(1H-imidazol-2-yl)-2-methylphenyl)-4-((5-(2-hydroxyethoxy)pyridin-2-yl)methoxy)benzamide

A mixture of N-(5-(1H-imidazol-2-yl)-2-methylphenyl)-4-((5-(2-(tert-butyldiphenylsilyloxy)ethoxy)pyridin-2-yl)methoxy)benzamide (15 mg, 0.02 mmol) in 1M TBAF in THF (1 mL) for 1 h. The mixture was concentrated in vacuo and the residue was purified with Gilson HPLC (2-65% MeCN/0.1% TFA in water) to yield the title compound (5.0 mg, 47.3%). ¹H NMR (DMSO-d₆) δ 14.70 (br s, 2H), 9.92 (s, 1H), 8.26 (d, 1H), 8.05 (d, 1H), 7.93 (m, 2H), 7.76-7.88 (m, 2H), 7.74 (s, 2H), 7.50 (d, 1H), 7.36-7.47 (m, 2H), 7.11 (m, 2H), 5.15 (s, 2H), 4.02 (t, 2H), 3.67 (d, 2H), 2.28 (s, 3H). MS (M+H⁺)=445.

### Example 132

### N-(5-(1H-imidazol-2-yl)-2-methylphenyl)-4-((4-(2-hydroxyethoxy)pyridin-2-yl)methoxy)benzamide

A microwave tube was charged with ethane-1,2-diol (222 mg, 3.58 mmol) and NaH (60% in mineral oil) (71.6 mg, 1.79 mmol). The mixture was stirred at RT for 1 h before N-(5-(1H-imidazol-2-yl)-2-methylphenyl)-4-((4-chloropyridin-2-yl)methoxy)benzamide (150 mg, 0.36 mmol) in 1 mL of DMF was added. The mixture was then subjected to microwave conditions for 30 min at 150° C. The tube was put back to microwave for 45 min at 150° C. The mixture was purified with Gilson HPLC (2-60% MeCN/0.1% TFA in water) to yield a white solid, which was repurified with Gilson HPLC (2-50% MeCN/10 mM NH₄OAc in water) to yield the title compound as a white solid (20.0 mg, 12.57%). ¹H NMR (DMSO-d₆) [symbol] 9.86 (s, 1H), 8.34 (d, 1H), 7.86-8.04 (m, 3H), 7.72 (dd, 1H), 7.52 (s, 2H), 7.43 (d, 1H), 7.12 (d, 2H), 6.97-7.08 (m, 2H), 6.90 (ddd, 3.16, 2H), 5.17 (s, 2H), 4.03 (t, 2H), 3.66 (t, 2H), 2.24 (s, 3H). MS (M+H⁺)=445.

### Example 133

### 4-[(2-cyanophenoxy)methyl]-N-[5-(1H-imidazol-2-yl)-2-methylphenyl]benzamide

### 133a. methyl 442-cyanophenoxy)methyl)benzoate

To a solution of 2-hydroxybenzonitrile (0.717 g, 6.02 mmol), methyl 4-(hydroxymethyl)benzoate (1 g, 6.02 mmol), and triphenylphosphine (2.53 g, 9.63 mmol) in THF (30 mL) was slowly added a DIAD (1.872 mL, 9.63 mmol) solution in THF (10 mL). The reaction was stirred overnight at RT and then concentrated in vacuo. The crude product was purified by ISCO MPLC (20-40% EtOAc/Hexanes) to give the title compound as a white solid (1.0 g, 62.2%). ¹H NMR (DMSO-d₆) δ 3.86 (br s, 3H) 5.40 (br s, 2H) 7.12 (br s, 1H) 7.33 (br s, 1H) 7.62 (d, 3H) 8.02 (d, 2H) 8.89 (br s, 1H). MS (M+H⁺) 268.

### 133b. 4-((2-cyanophenoxy)methyl)benzoic acid

methyl 4-((2-cyanophenoxy)methyl)benzoate (1 g, 3.74 mmol) was dissolved in MeOH (20 mL) with NaOH (15 mL, 15.0 mmol). The reaction mixture was stirred overnight at RT and then concentrated by removal of the MeOH. The resulting aqueous solution was acidified with HCl and the precipitate filtered to yield the title compound (0.948 g, 100%). ¹H NMR (DMSO-d₆) δ 5.39 (s, 2H) 7.12 (t, 1H) 7.32 (d, 1H) 7.55-7.71 (m, 3H) 7.77 (d, 1H) 7.99 (d, 2H) 12.99 (br s, 1H). MS (M−H⁺), 252.

### 133c. N-(5-(1H-imidazol-2-yl)-2-methylphenyl)-4-((2-cyanophenoxy)methyl)benzamide

4-((2-cyanophenoxy)methyl)benzoic acid (250 mg, 0.99 mmol) was diluted with SOCl₂ (5 mL) and a few drops of DMF were added. The reaction was stirred overnight at RT and then concentrated in vacuo, redissolved in DCM and then concentrated once more to give 4-[(2-cyanophenoxy)methyl]benzoyl chloride. To the acid chloride dissolved in pyridine (2 mL) and DCM (2 mL), was added 5-(1H-imidazol-2-yl)-2-methylaniline (171 mg, 0.99 mmol). The reaction was stirred overnight at RT and then heated to 50° C. for 4 h. After cooling to RT, the mixture was poured onto water and then extracted into EtOAc (3×50 mL) and then washed with brine and dried over Na₂SO₄, filtered and concentrated in vacuo. The crude product was purified by ISCO MPLC (2-5% MeOH/DCM) to give desired product but impure. The residue was repurified using Gilson HPLC (5-95% MeCN/10 mM NH₄OAc in water) to give the title compound (48.0 mg, 11.90%). ¹H NMR (DMSO-d₆) δ 2.26 (s, 3H) 5.41 (s, 2H) 7.00 (s, 1H) 7.13 (dd, 1H) 7.22 (s, 1H) 7.35 (dd, 2H) 7.67-7.71 (m, 3H) 7.76 (dd, 2H) 7.94 (s, 1H) 8.05 (d, 2H) 10.02 (s, 1H) 12.47 (br s, 1H). MS (M+H⁺) 409.

The following Example 134 was prepared in a similar fashion to Example 133 utilizing commercially available starting materials:

| | | | MS | |
|---|---|---|---|---|
| Ex. | Name | MW | (M + H⁺) | ¹H NMR ( [symbol] ppm) |
| 134 | N-[5-(1H-imidazol-2-yl)- | 384.44 | 385 | 2.25 (s, 3 H) 5.46 (s, 2 H) 6.93 (d, 1 H) |
| | 2-methylphenyl]-4- | | | 6.97-7.05 (m, 1 H) 7.11 (br s, 2 H) |
| | [(pyridin-2- | | | 7.34 (d, 1 H) 7.60 (m, 2 H) 7.67-7.82 (m, 2 H) |
| | yloxy)methyl]benzamide | | | 7.93 (s, 1 H) 8.01 (m, 2 H) 8.11-8.26 (m, |
| | | | | 1 H) 9.99 (s, 1 H) 12.47 (br s, 1 H) |

### Example 135

### N-(5-(1H-imidazol-2-yl)-2-methylphenyl)-4-((3-methoxypyridin-2-yl)methoxy)benzamide

### 135a. (3-methoxypyridin-2-yl)-methanol

To a flask charged with 3-fluoropicolinaldehyde (400 mg, 3.20 mmol) was added sodium methoxide (15 mL, 7.50 mmol) (0.5M in MeOH). The mixture was stirred at 80° C. for 4 h. The reaction mixture was then cooled to 0° C. with an ice bath and NaBH₄ (90 mg, 2.38 mmol) was added to the mixture was stirred at 0° C. for 20 min before ice was added to the mixture. After concentration in vacuo, the residue was purified with ISCO MPLC (40-100% EtOAc/hexane) to yield the title compound as a white solid (200 mg, 45.0%). ¹H NMR (DMSO-d₆) [symbol] 8.11 (dd, 1H), 7.41 (dd, 1H), 7.31 (dd, 1H), 4.83 (t, 1H), 4.54 (d, 2H), 3.82 (s, 3H). MS (M+H⁺)=140.

### 135b. (3-methoxypyridin-2-yl)-methyl 4-methylbenzenesulfonate

Prepared in a similar fashion to (6-bromopyridin-2-yl)methyl 4-methylbenzenesulfonate (Example 119, step a). MS (M+H⁺)=293.

### 135c. N-(5-(1H-imidazol-2-yl)-2-methylphenyl)-4-((3-methoxypyridin-2-yl)methoxy)benzamide

Prepared in a similar fashion to Example 119. ¹H NMR (DMSO-d₆) [symbol] 12.38 (br s, 1H), 9.76 (s, 1H), 8.10 (d, 1H), 7.91 (d, 2H), 7.84 (d, 1H), 7.66 (dd, 1H), 7.47 (d, 1H), 7.35 (dd, 1H), 7.26 (d, 1H), 7.08 (d, 4H), 5.16 (s, 2H), 3.81 (s, 3H), 2.17 (s, 3H). MS (M+H⁺)=415.

### Example 136

### N-(5-(1H-imidazol-2-yl)-2-methylphenyl)-4-((3-cyanopyridin-2-yl)methoxy)benzamide

### 136a. 2-(bromomethyl)nicotinonitrile

A mixture of 2-methylnicotinonitrile (365 mg, 3.09 mmol), N-bromosuccinimide (660 mg, 3.71 mmol) and AIBN (20.29 mg, 0.12 mmol) in CCl₄ (10 mL) was stirred at 80° C. for 4 h. After concentration in vacuo the residue was purified with ISCO MPLC (10-50% EtOAc/hexane) to yield the title compound as a yellow oil (224 mg, 36.8%). ¹H NMR CDCl₃) [symbol] 8.81 (dd, 1H), 8.02 (dd, 1H), 7.41 (dd, 1H), 4.75 (s, 2H). MS (M+H⁺)=196, 198.

### 136b. N-(5-(1H-imidazol-2-yl)-2-methylphenyl)-4-((3-cyanopyridin-2-yl)methoxy)benzamide

Prepared in a similar fashion to Example 98. ¹H NMR (DMSO-d₆) [symbol] 15.06 (br s, 2H), 10.10 (s, 1H), 8.88 (dd, 11H), 8.43 (dd, 1H), 8.16 (d, 1H), 7.96-8.11 (m, 3H), 7.78 (s, 2H), 7.66 (dd, 1H), 7.56 (d, 1H), 7.21 (d, 2H), 5.46 (s, 2H), 2.34 (s, 3H). MS (M+H⁺)=410.

### Example 137

### N-(5-(1H-imidazol-2-yl)-2-methylphenyl)-4-((3-morpholinopyridin-2-yl)methoxy)benzamide

### 137a. 3-morpholinopicolinaldehyde

A mixture of 3-fluoropicolinaldehyde (400 mg, 3.20 mmol), morpholine (557 mg, 6.39 mmol), K₂CO₃ (1326 mg, 9.59 mmol) in DMF (3 mL) was stirred at 80° C. for 3 h. Sat. NaHCO₃ was added to the mixture, and then extracted with EtOAc (3×) The combined organic layers were dried over anhydrous Na₂SO₄, filtered, and concentrated in vacuo. The residue was purified with ISCO MPLC (30-100% EtOAc/hexane) to yield the title compound as a yellow solid (400 mg, 65.1%). MS (M+H⁺)=193.

### 137b. (3-morpholinopyridin-2-yl)methanol

A mixture of 3-morpholinopicolinaldehyde (400 mg, 2.08 mmol) in MeOH (15 mL) was cooled to 0° C. NaBH₄ (55.1 mg, 1.46 mmol) was added in one portion. The mixture was stirred at RT for 10 min after which 2N NaOH (1 mL) was added to the mixture. After concentration in vacuo, the residue was purified with ISCO MPLC (40-100% EtOAc/hexane) to yield the title compound as a white solid (398 mg, 98%). ¹HNMR (DMSO-d₆) 8.25 (d, 1H), 7.50 (d, 1H), 7.29 (m, 1H), 5.00 (t, 1H), 4.56 (d, 2H), 3.78 (m, 4H), 3.89 (m, 4H). MS (M+H⁺)=195.

### 137c. 4-(2-(chloromethyl)pyridin-3-yl)morpholine

To a mixture of (3-morpholinopyridin-2-yl)methanol (50 mg, 0.26 mmol), 4-methylbenzene-1-sulfonyl chloride (54.0 mg, 0.28 mmol) in DCM (5 mL) was added TEA (52.1 mg, 0.51 mmol) and DMAP (5 mg, 0.04 mmol) at RT. The mixture was stirred at RT for 3 h. sat. NH₄Cl was added to the mixture and extracted with DCM (2×). The combined organic layers were dried over anhydrous Na₂SO₄, filtered, and concentrated in vacuo to yield the title compound. MS (M+H⁺)=213.

### 137d. N-(5-(1H-imidazol-2-yl)-2-methylphenyl)-4-((3-morpholinopyridin-2-yl)methoxy)benzamide

Prepared in a similar fashion to Example 97. ¹H NMR (DMSO-d₆) [symbol] 14.72 (br s, 2H), 9.93 (s, 1H), 8.32 (d, 1H), 8.05 (d, 1H), 7.94 (m, 2H), 7.83 (dd, 1H), 7.74 (s, 3H), 7.39-7.57 (m, 2H), 7.13 (m, 2H), 5.28 (s, 2H), 3.67-3.69 (m, 4H), 2.84-2.98 (m, 4H), 2.24-2.31 (m, 3H). MS (M+H⁺)=470.

### Example 138

### N-(5-(1H-imidazol-2-yl)-2-methylphenyl)-4-((3-(4-methylpiperazin-1-yl)pyridin-2-yl)methoxy)benzamide

### 138a. methyl 4-((3-bromopyridin-2-yl)methoxy)benzoate

Prepared in a similar fashion to example 97. ¹H NMR (CDCl₃) [symbol] 8.52 (dd, 1H), 7.90-8.02 (m, 3H), 7.87 (dd, 1H), 7.13 (dd, 1H), 6.92-7.03 (m, 2H), 5.30 (s, 2H), 3.82 (s, 3H). MS (M+H⁺)=323.

### 138b. methyl 4-((3-(4-methylpiperazin-1-yl)pyridin-2-yl)methoxy)benzoate

A mixture of methyl 4-((3-bromopyridin-2-yl)methoxy)benzoate (360 mg, 1.12 mmol), Pd₂ dba₃ (205 mg, 0.22 mmol), BINAP (278 mg, 0.45 mmol), Cs₂CO₃ (728 mg, 2.23 mmol), and 1-methylpiperazine (168 mg, 1.68 mmol) in DMA (10 mL) was stirred at 100° C. overnight. Sat. NaHCO₃ was added to the mixture and extracted with EtOAc (3×). The combined organics were dried over anhydrous Na₂SO₄, filtered, and concentrated in vacuo. The residue was purified with ISCO MPLC (50-100% EtOAc/hexane to 40% MeOH/EtOAc) to yield the title compound as a brown oil (245 mg, 64.2%). MS (M+H⁺)=342.

### 138c. 4-((3-(4-methylpiperazin-1-yl)pyridin-2-yl)methoxy)benzoic acid

To a mixture of methyl 4-((3-(4-methylpiperazin-1-yl)pyridin-2-yl)methoxy)benzoate (245 mg, 0.72 mmol) in MeOH (2.0 mL), THF (4 mL) and water (1.0 mL) was added LiOH (25.8 mg, 1.08 mmol). The mixture was stirred at RT for 4 h. Additional LiOH (180 mg) was added to the reaction mixture and stirred at RT overnight. Concentration in vacuo afforded the title compound. MS (M+H⁺)=328.

### 138d. N-(5-(1H-imidazol-2-yl)-2-methylphenyl)-4-((3-(4-methylpiperazin-1-yl)pyridin-2-yl)methoxy)benzamide

A mixture of 4-((3-(4-methylpiperazin-1-yl)pyridin-2-yl)methoxy)benzoic acid (100 mg, 0.31 mmol) in SOCl₂ (10 mL, 137.01 mmol) was stirred at reflux for 2 h. After concentration in vacuo, the residue was diluted with DCM and concentrated in vacuo again to give 4-{[3-(4-methylpiperazin-1-yl)pyridin-2-yl]methoxy}benzoyl chloride. The residue was mixed with 5-(1H-imidazol-2-yl)-2-methylaniline (52.9 mg, 0.31 mmol) and dissolved in a mixture of DCM (10 mL) and pyridine (10 mL). The mixture was stirred at 50° C. for 0.5 h. After concentration in vacuo, the residue was purified with ISCO MPLC (0-90% MeOH/EtOAc) to yield a crude product that was repurified with Gilson HPLC (5-50% MeCN/10 mM NH₄OAc in water) to yield the title compound as a white solid (34.0 mg, 23.07%). ¹H NMR (DMSO-d₆) [symbol] 10.45 (s, 1H), 8.91 (dd, 1H), 8.58 (d, 2H), 8.51 (d, 1H), 8.33 (dd, 1H), 8.22 (dd, 1H), 7.82-8.04 (m, 2H), 7.56-7.82 (m, 4H), 5.86 (s, 2H), 3.57 (t, 4H), 3.11 (d, 4H), 2.84 (d, 6H). MS (M+H⁺)=483.

### Example 139

### N-(5-(1H-imidazol-2-yl)-2-methylphenyl)-4-((5-morpholinopyridin-2-yl)methoxy)benzamide

### 139a. 5-morpholinopicolinaldehyde

A 50 mL round bottom flask was charged with a magnetic stir bar and 5-fluoropicolinaldehyde (0.751 g, 6.0 mmol). MeCN (16 mL), morpholine (1.046 mL, 12.01 mmol) and K₂CO₃ (1.659 g, 12.01 mmol) were added and the reaction was heated to reflux for 4 h. The reaction was then allowed to cool to RT, filtered through Celite, and concentrated in vacuo to afford the crude product which was purified via ISCO MPLC (EtOAc) to afford the title compound (0.499 g, 43.2%). ¹H NMR (DMSO-d₆) [symbol] 9.77 (s, 1H) 8.47 (s, 1H) 7.77 (d, 1H) 7.41 (d, 1H) 3.74 (t, 4H) 3.41 (t, 4H). MS (M+H⁺)=193.

### 139b. (5-morpholinopyridin-2-yl)methanol

A 200 mL round bottom flask was charged with a magnetic stir bar, 5-morpholinopicolinaldehyde (0.499 g, 2.60 mmol), and anhydrous MeOH (10.38 mL). The vessel was cooled with an ice bath and NaBH₄ (0.147 g, 3.89 mmol) was added in a single portion. The reaction was placed under nitrogen and allowed to stir at 0° C. for 15 min before the careful addition of 1N NaOH (10 mL). The resulting mixture was stirred for 15 min at this temperature and was then extracted with EtOAc (3×30 mL). The combined organic phase was dried with MgSO₄, filtered, and concentrated in vacuo to afford the title compound (0.350 g, 69.4%) as an off white solid. ¹H NMR (DMSO-d₆) δ 8.18 (s, 1H) 7.36-7.26 (m, 3H) 5.20 (t, 1H) 4.44 (d, 1H) 3.73 (t, 4H) 3.11 (t, 4H). MS (M+H⁺)=195.

### 139c. 4-((5-morpholinopyridin-2-yl)methoxy)benzonitrile

A 100 mL round bottom flask was charged with a magnetic stir bar, (5-morpholinopyridin-2-yl)methanol (269 mg, 1.38 mmol), and anhydrous DMF (5.149 mL). NaH (60% in mineral oil) (69.2 mg, 1.73 mmol) was added and the mixture was allowed to stir for 15 min before the addition of 4-fluorobenzonitrile (210 mg, 1.73 mmol). The mixture was allowed to stir overnight at RT followed by the careful addition of dilute aq. NH₄Cl (˜50 mL). Additional water (˜50 mL) was added and a precipitate formed which was collected via vacuum filtration. The filter cake was washed with water (˜50 mL), collected, and dried in vacuo to afford the title compound (355 mg, 87%) as a pale yellow solid. ¹H NMR (DMSO-d₆) δ 8.29 (s, 1H) 7.76 (d, 2H) 7.36 (s, 2H) 7.17 (d, 2H) 5.14 (d, 2H) 3.73 (t, 4H) 3.13 (t, 4H). MS (M+H⁺)=296.

### 139d. 4-((5-morpholinopyridin-2-yl)methoxy)benzoic acid

A 100 mL round bottom flask was charged with a magnetic stir bar, 4-((5-morpholinopyridin-2-yl)methoxy)benzonitrile (345 mg, 1.17 mmol), EtOH (3.70 mL), water (0.935 mL), and NaOH (93 mg, 2.34 mmol). The mixture was heated to reflux with stirring overnight before being allowed to cool to RT and concentrated in vacuo. The crude solid was suspended in 10% HCl until a pH of ˜2 was achieved. The mixture was filtered and the filter cake was washed with water (3×10 mL). The filter cake was collected and dried in vacuo to give the title compound as the hydrochloride salt (240 mg, 58.6%). ¹H NMR (DMSO-d₆) δ 12.67 (br s 1H) 8.35 (s, 1H) 7.90 (d, 2H) 7.70-7.62 (m, 2H) 7.10 (d, 2H) 5.25 (s, 2H) 3.74 (t, 4H) 3.26 (t, 4H). MS (M+H⁺)=315.

### 139e. N-(5-(1H-imidazol-2-yl)-2-methylphenyl)-4-((5-morpholinopyridin-2-yl)methoxy)benzamide

A 100 mL round bottom flask was charged with a magnetic stir bar, 4-((5-morpholinopyridin-2-yl)methoxy)benzoic acid hydrochloride (83 mg, 0.24 mmol), 5-(1H-imidazol-2-yl)-2-methylaniline hydrochloride (49.6 mg, 0.24 mmol), DMF (0.70 mL), DIPEA (0.250 mL, 1.42 mmol), and HATU (135 mg, 0.35 mmol). The mixture was heated to 50° C. in an oil bath with stirring for 4 h before being cooled to RT. Water (˜50 mL) was added and the mixture was extracted with EtOAc (2×50 mL). The combined organic extract was washed with brine (˜100 mL), dried over MgSO₄, filtered through a bed of Celite, and concentrated in vacuo to yield the crude product, which was purified via Gilson HPLC (5-55% MeCN/0.1% TFA in water). The material was diluted with HCl and concentrated in vacuo to afford the hydrochloride salt of the title compound (55.0 mg, 45.9%) as an off white solid. ¹H NMR (DMSO-d₆) δ 10.00 (s, 1H) 8.37 (s, 1H) 8.13 (s, 1H) 8.02 (d, 2H) 7.79-7.77 (m, 4H) 7.55 (d, 1H) 7.18 (d, 2H) 5.31 (s, 2H) 3.76-3.73 (m, 4H) 3.30-3.27 (m, 4H) 2.33 (s, 3H). MS (M+H⁺)=470.

### Example 140

### N-(5-(1H-imidazol-2-yl)-2-methylphenyl)-4-((5-morpholinopyridin-2-yl)methylamino)benzamide

### 140a. tert-butyl 4-(5-(1H-imidazol-2-yl)-2-methylphenylcarbamoyl)phenylcarbamate

A 50 mL round bottom flask was charged with a magnetic stir bar, 4-(tert-butoxycarbonylamino)benzoic acid (0.300 g, 1.26 mmol), 5-(1H-imidazol-2-yl)-2-methylaniline hydrochloride (0.265 g, 1.26 mmol), DMF (3.11 mL), and DIPEA (1.104 mL, 6.32 mmol). HATU (0.721 g, 1.90 mmol) was added and the reaction was warmed to 50° C. with stirring for 6 h. The reaction was allowed to cool to RT and was poured into brine (˜50 mL) and was extracted with EtOAc (2×50 mL). The combined organic phase was washed with brine, dried with MgSO₄, filtered, and concentrated in vacuo to yield the crude product which was purified using ISCO MPLC (EtOAc) to afford the title compound (0.210 g, 42.3%) as an off white solid. ¹H NMR (DMSO-d₆) δ 12.43 (s, 1H) 9.82 (s, 1H) 9.69 (s, 1H) 7.94-7.89 (m, 2H) 7.71 (d, 1H) 7.58 (d, 2H) 7.32 (d, 1H) 7.19 (s, 1H) 6.99 (s, 1H) 2.23 (s, 3H) 1.49 (s, 9H). MS (M+H⁺)=393.

### 140b. N-(5-(1H-imidazol-2-yl)-2-methylphenyl)-4-aminobenzamide

A 100 mL round bottom flask was charged with a magnetic stir bar, tert-butyl 4-(5-(1H-imidazol-2-yl)-2-methylphenylcarbamoyl)phenylcarbamate (200 mg, 0.51 mmol), and HCl (4N) in Dioxane (5 mL, 144 mmol). The mixture was stirred at RT for 2 h and was then concentrated in vacuo to afford the hydrochloride salt of the title compound (147 mg, 88%) as an off white solid. ¹H NMR (DMSO-d₆) δ 15.01 (br s, 1H) 9.98 (s, 1H) 8.14 (s, 1H) 7.99 (d, 1H) 7.92 (d, 2H) 7.77 (s, 2H) 7.53 (d, 2H) 7.04 (br s, 2H) 2.32 (s, 3H). MS (M+H⁺)=293.

### 140c. N-(5-(1H-imidazol-2-yl)-2-methylphenyl)-4-((5-morpholinopyridin-2-yl)methylamino)benzamide

A 100 mL round bottom flask was charged with a magnetic stir bar, N-(5-(1H-imidazol-2-yl)-2-methylphenyl)-4-aminobenzamide hydrochloride (125 mg, 0.38 mmol), 5-morpholinopicolinaldehyde (73.1 mg, 0.38 mmol), and DCM (3.80 mL). Sodium triacetoxyborohydride (322 mg, 1.52 mmol) was added and the mixture was allowed to stir for 4 h at RT before being concentrated in vacuo. The obtained solid was dissolved in DMSO (˜3 mL) and purified via Gilson HPLC (5-75% MeCN/0.1% TFA in water) to afford pure material that was treated with a 4N HCl solution in dioxane (5 mL) and concentrated in vacuo to afford the title compound as the hydrochloride salt (75 mg, 39.1%). ¹H NMR (DMSO-d₆) δ 10.05 (s, 1H) 8.37 (s, 1H) 8.13 (s, 1H) 8.04 (d, 2H) 7.80-7.74 (m, 3H) 7.55 (d, 1H) 7.18 (d, 2H) 5.31 (s, 2H) 3.75 (t, 4H) 3.29 (br s, 4H) 2.33 (s, 3H). MS (M+H⁺)=469.

### Example 141

### tert-butyl 2-((4-(5-(1H-imidazol-2-yl)-2-methylphenylcarbamoyl)phenoxy)methyl)piperidine-1-carboxylate

A 50 mL vial was charged with a magnetic stir bar, N-(5-(1H-imidazol-2-yl)-2-methylphenyl)-4-hydroxybenzamide (170 mg, 0.58 mmol), tert-butyl 2-(bromomethyl)piperidine-1-carboxylate (177 mg, 0.64 mmol), K₂CO₃ (200 mg, 1.45 mmol), MeCN (2 mL), NaI (˜25 mg) and water (200 μL). The mixture was heated to 80° C. with stirring for 72 h before being allowed to cool to RT. The mixture was purified by ISCO MPLC (20% MeOH/EtOAc) to afford the title compound (179 mg, 63.0%) as an off white solid. ¹H NMR (DMSO-d₆) δ 12.44 (s, 1H) 9.83 (s, 1H) 7.98 (d, 2H) 7.89 (s, 1H) 7.70 (d, 1H) 7.32 (d, 1H) 7.06 (d, 2H) 3.97-3.91 (m, 2H) 2.90-2.88 (m, 1H) 2.23 (s, 3H) 1.98-1.62 (m, 4H) 1.36 (s, 9H) 0.99-0.82 (m, 4H). MS (M+H⁺)=491.

### Example 142

### N-(5-(1H-imidazol-2-yl)-2-methylphenyl)-4-(piperidin-2-ylmethoxy)benzamide

A 50 mL round bottom flask was charged with a magnetic stir bar, tert-butyl 2-((4-(5-(1H-imidazol-2-yl)-2-methylphenylcarbamoyl)phenoxy)methyl)piperidine-1-carboxylate (111 mg, 0.23 mmol), MeOH (5 mL), and HCl (4N in dioxane, 4 mL, 115.19 mmol). The mixture was allowed to stir for 2 h at RT before being concentrated in vacuo affording the title compound (91 mg, 94%) as its hydrochloride salt. ¹H NMR (DMSO-d₆) δ 14.91 (br s, 2H) 10.04 (s, 1H) 9.12 (br s, 1H) 9.10 (br s 1H) 8.12-7.98 (m, 3H) 7.77 (s, 2H) 7.54 (d, 1H) 7.09 (d, 2H) 4.06-3.97 (m, 2H) 3.93 (d, 1H) 3.50-3.34 (m, 4H) 2.77 (t, 2H) 2.32 (s, 3H) 1.86-1.67 (m, 4H). MS (M+H⁺)=391.

### Example 143

### N-(5-(1H-imidazol-2-yl)-2-methylphenyl)-4-((1-methylpiperidin-2-yl)methoxy)benzamide

A 50 mL round bottom flask was charged with a magnetic stir bar, N-(5-(1H-imidazol-2-yl)-2-methylphenyl)-4-(piperidin-2-ylmethoxy)benzamide hydrochloride (115 mg, 0.27 mmol), MeCN (1.5 mL), and formaldehyde solution (0.5 mL) (in water). The mixture was allowed to stir for 10 min and then sodium cyanoborohydride (42.3 mg, 0.67 mmol) was added and the mixture was allowed to stir for 1 h at RT. The mixture was concentrated in vacuo and purified by ISCO MPLC (20% MeOH/DCM) to afford the pure material which was dissolved in MeOH (5 mL), treated with 4N HCl in dioxane (5 mL) and concentrated in vacuo to afford the hydrochloride salt of the title compound (101 mg, 79%) as a white solid. ¹H NMR (DMSO-d₆) δ 15.12 (br s, 1H) 11.07 (s, 1H) 10.10 (s, 1H) 8.16 (s, 1H) 8.07-8.04 (m, 3H) 7.75 (s, 2H) 7.53 (d, 1H) 7.08 (d, 2H) 4.08-3.93 (m, 2H) 3.58-3.48 (m, 4H) 2.79-2.73 (m, 2H) 2.72 (s, 3H) 2.32 (s, 3H) 1.88-1.85 (m, 2H). MS (M+H⁺)=405.

### Example 144

### N-(5-(1H-imidazol-2-yl)-2-methylphenyl)-4-((3-hydroxypyridin-2-yl)methoxy)benzamide

### 144a. 3-(tert-butyldiphenylsilyloxy)-2-methylpyridine

To a mixture of 2-methylpyridin-3-ol (1 g, 9.16 mmol), tert-butylchlorodiphenylsilane (3.52 mL, 13.75 mmol) in DMF (10 mL) was added 1H-imidazole (1.560 g, 22.91 mmol). The mixture was stirred at RT for 20 min and after concentrating in vacuo, the residue was purified by ISCO MPLC (10-20% EtOAc/hexane) to yield the title compound as a colorless oil which turned into a white solid after standing at RT overnight (2.33 g, 73.1%). ¹H NMR (CDCl₃) δ 7.94 (dd, 1H), 7.55-7.67 (m, 4H), 7.34-7.42 (m, 2H), 7.25-7.34 (m, 4H), 6.59-6.71 (m, 1H), 6.49-6.59 (m, 1H), 2.58 (s, 3H), 1.05 (s, 9H). MS (M+H⁺)=348

### 144b. 2-(bromomethyl)-3-(tert-butyldiphenylsilyloxy)pyridine

A mixture of 3-(tert-butyldiphenylsilyloxy)-2-methylpyridine (2 g, 5.75 mmol), 1-bromopyrrolidine-2,5-dione (1.178 g, 6.62 mmol), (E)-2,2′-(diazene-1,2-diyl)bis(2-methylpropanenitrile) (0.189 g, 1.15 mmol) in CCl₄ (20 mL) was stirred at 80° C. for 5 h. After concentration in vacuo, the residue was purified with ISCO MPLC (20% EtOAc/hexane) to afford the title compound as a colorless oil which turned into a white solid after standing at RT for 2 h (1.360 g, 55.4%). ¹H NMR (CDCl₃) δ 8.03 (dd, 1H), 7.53-7.73 (m, 4H), 7.35-7.44 (m, 2H), 7.27-7.35 (m, 4H), 6.77 (dd, 1H), 6.62 (d, 1H), 4.76 (s, 2H), 1.08 (s, 9H). MS (M+H⁺)=428.

### 144c. N-(5-(1H-imidazol-2-yl)-2-methylphenyl)-4-((3-hydroxypyridin-2-yl)methoxy)benzamide

A mixture of 2-(bromomethyl)-3-(tert-butyldiphenylsilyloxy)pyridine (600 mg, 1.41 mmol), N-(5-(1H-imidazol-2-yl)-2-methylphenyl)-4-hydroxybenzamide (413 mg, 1.41 mmol) and K₂CO₃ (778 mg, 5.63 mmol) in acetonitrile (15 mL) and water (0.50 mL) was stirred at 80° C. for 4 h. The reaction was then stirred at 60° C. overnight. After cooling to RT, the reaction was filtered, washed with methanol, and concentrated in vacuo. The residue was purified by Gilson HPLC (10-45% MeCN/10 mM NH₄OAc in water) to afford the title compound as a white solid (130 mg, 23.07%). ¹H NMR (DMSO-d₆) [symbol] 9.63 (s, 1H), 7.88 (dd, 1H), 7.78 (m, 2H), 7.62 (d, 1H), 7.37 (dd, 1H), 7.24 (d, 1H), 7.13-7.20 (m, 1H), 7.06-7.13 (m, 1H), 7.04 (s, 1H), 6.88 (s, 1H), 6.79 (m, 2H), 5.23 (s, 2H), 2.18 (s, 3H), 1.78 (s, 2H). MS (M+H⁺)=401.

### Example 145

### N-[5-(1H-imidazol-2-yl)-2-methyl-phenyl]-6-phenoxy-pyridine-3-carboxamide

### 145a. 5-(1H-imidazol-2-yl)-2-methylaniline

In a 10 mL vial was added 2-bromo-1H-imidazole (3.08 g, 20.95 mmol), 2-methyl-5-(4,4,5,5-tetramethyl-1,3,2-dioxaborolan-2-yl)aniline (3.0 g, 13.09 mmol), and K₂CO₃ (3.85 g, 39.27 mmol) in dioxane (6.0 mL) to give a colorless suspension. The reaction mixture was diluted with water (1.5 mL). After bubbling through N₂ for 20 min, Pd(PPh₃)₄ (2.269 g, 1.96 mmol) was added. The reaction was heated at 110° C. for 50 h. The solvents were removed under reduced pressure and the residue was purified by ISCO MPLC (10% MeOH/DCM) to give the title compound (0.68 g, 30% yield). ¹H NMR (DMSO-d₆) δ 2.07 (s, 3H), 4.91 (br. s, 2H), 6.98 (m, 3H), 7.21 (s, 1H), 7.65 (s, 1H), 12.13 (br s br s, 1H). MS (M+H⁺)=173.

### 145b. N-[5-(1H-imidazol-2-yl)-2-methyl-phenyl]-6-phenoxy-pyridine-3-carboxamide

In a 20 mL vial was added 5-(1H-imidazol-2-yl)-2-methylaniline (0.07 g, 0.40 mmol) in pyridine (1.0 mL) to give a yellow suspension. 6-phenoxynicotinoyl chloride (0.103 g, 0.44 mmol) was added and the reaction was stirred at RT overnight. After concentration under reduced pressure, the solution was purified by Gilson HPLC (MeCN/10 mM NH₄OAc in water) to give the title compound (0.011 g, 7.5% yield). ¹H NMR (DMSO-d₆) δ 2.25 (s, 4H), 7.00 (s, 1H), 7.20 (d, 3H), 7.27 (t, 1H), 7.35 (d, 1H), 7.47 (t, 2H), 7.74 (dd, 1H), 7.92 (s, 1H), 8.39 (dd, 1H), 8.77 (d, 1H), 10.07 (s, 1H), 12.47 (br. s., 1H). MS (M+H⁺)=371.

The following Examples 146-154 were prepared in a similar fashion to Example 145 utilizing commercially available starting materials:

| | | | MS | |
|---|---|---|---|---|
| Ex. | Name | MW | (M + H⁺) | ¹H NMR (δ ppm) |
| 146 | 3-cyano-N-[5-(1H- | 302.34 | 303 | 2.27 (s, 3 H), 7.16 (s, 2 H), 7.37 (d, 1 H), |
| | imidazol-2-yl)-2-methyl- | | | 7.77 (m, 2 H), 7.94 (s, 1 H), 8.10 (d, 1 H), |
| | phenyl]benzamide | | | 8.30 (d, 1 H), 8.43 (s, 1 H), 10.22 (s, 1 H), |
| | | | | 12.75 (s, 1 H) |
| 147 | N-[5-(1H-imidazol-2-yl)- | 360.3 | 361 | 2.32 (s, 3 H), 2.72 (s, 3 H), 7.21 (s, 2 H), |
| | 2-methyl-phenyl]-2- | | | 7.38 (d, 1 H), 7.75 (m, 1 H), 7.91 (d, 1 H), |
| | methyl-6- | | | 8.08 (s, 1 H), 8.22 (d, 1 H), 10.21 (s, 1 H), |
| | (trifluoromethyl)pyridine- | | | 13.00 (s, 1 H) |
| | 3-carboxamide | | | |
| 148 | 3-dimethylamino-N-[5- | 320.4 | 321 | 2.25 (s, 3 H), 2.97 (s, 6 H), 6.93 (m, 1 H), |
| | (1H-imidazol-2-yl)-2- | | | 7.00 (s, 1 H), 7.21 (s, 1 H), 7.31 (m, 4 H), |
| | methyl-phenyl]benzamide | | | 7.74 (dd, H), 7.91 (s, 1 H), 9.89 (s, 1 H), |
| | | | | 12.47 (s, 1 H) |
| 149 | N-[5-(1H-imidazol-2-yl)- | 348.4 | 349 | 2.22 (s, 3 H), 2.93 (s, 3 H), 3.34 (m, 2 H), |
| | 2-methyl-phenyl]-2- | | | 4.25 (m, 2 H), 6.76 (d, 1 H), 6.99 (s, 1 H), |
| | methyl-5-oxa-2- | | | 7.20 (s, 1 H), 7.31 (d, 1 H), 7.37 (d, 1 H), |
| | azabicyclo[4.4.0]deca- | | | 7.53 (dd, 1 H), 7.70 (dd, 1 H), 7.88 (s, 1 |
| | 7,9,11-triene-8- | | | H), 9.59 (s, 1 H), 12.44 (s, 1 H) |
| | carboxamide | | | |
| 150 | N-[5-(1H-imidazol-2-yl)- | 346.3 | 347 | 2.28 (s, 3 H), 7.01 (s, 1 H), 7.23 (s, 1 H), |
| | 2-methyl-phenyl]-6- | | | 7.38 (dd, 1 H), 7.77 (dt, 1 H), 7.98 (s, 1 |
| | (trifluoromethyl)pyridine- | | | H), 8.14 (dd, 1 H), 8.60 (s, 1 H), 9.31 (s, 1 |
| | 3-carboxamide | | | H), 10.41 (s, 1 H), 12.50 (s, 1 H) |
| 151 | N-[5-(1H-imidazol-2-yl)- | 335.4 | 336 | 2.23 (s, 3 H), 4.32 (m, 4 H), 6.99 (d, 2 H), |
| | 2-methyl-phenyl]-2,5- | | | 7.19 (s, 1 H), 7.32 (d, 1 H), 7.53 (m, 2 H), |
| | dioxabicyclo[4.4.0]deca- | | | 7.72 (dd, 1 H), 7.88 (s, 1 H), 9.81 (s, 1 H), |
| | 7,9,11-triene-8- | | | 12.45 (s, 1 H) |
| | carboxamide | | | |
| 152 | 3-chloro-N-[5-(1H- | 311.8 | 312 | 2.25 (s, 3 H), 7.00 (s, 1 H), 7.19 (s, 1 H), |
| | imidazol-2-yl)-2-methyl- | | | 7.36 (s, 1 H), 7.59 (s, 1 H), 7.69 (s, 1 H), |
| | phenyl]benzamide | | | 7.75 (s, 1 H), 7.91 (s, 1 H), 7.96 (s, 1 H), |
| | | | | 8.04 (s, 1 H), 10.13 (s, 1 H), 12.48 (s, 1 H) |
| 153 | N-[5-(1H-imidazol-2-yl)- | 363.4 | 364 | 2.24 (s, 3 H), 3.61 (q, 4 H), 3.71 (m, 4 H), |
| | 2-methyl-phenyl]-6- | | | 6.94 (d, 1 H), 7.10 (s, 2 H), 7.33 (d, 1 H), |
| | morpholin-4-yl-pyridine- | | | 7.73 (dd, 1 H), 7.91 (s, 1 H), 8.12 (dd, 1 |
| | 3-carboxamide | | | H), 8.79 (d, 1 H), 9.79 (s, 1 H) |
| 154 | N-[5-(1H-imidazol-2-yl)- | 337.4 | 338 | 2.25 (s, 3 H), 3.84 (s, 6 H), 7.11 (m, 3 H), |
| | 2-methyl-phenyl]-3,4- | | | 7.35 (d, 1 H), 7.59 (d, 1 H), 7.66 (dd, 1 |
| | dimethoxy-benzamide | | | H), 7.74 (dd, 1 H), 7.90 (s, 1 H), 9.88 (s, |
| | | | | 1 H), 12.62 (s, 1 H) |

The following Example 155 was prepared in a similar fashion to Example 139 utilizing commercially available starting materials:

| | | | MS | |
|---|---|---|---|---|
| Ex. | Name | MW | (M + H⁺) | ¹H NMR ( [symbol] ppm) |
| 155 | N-[5-(1H-imidazol-2-yl)- | 482.59 | 483 | 14.97 (br s, 1 H), 11.20 (br s, 1 H), 10.07 (s, |
| | 2-methylphenyl]-4-{[5-(4- | | | H), 8.45 (s, 1 H), 8.14 (s, 1 H), 8.02 (d, 2 H) |
| | methylpiperazin-1- | | | 7.99 (m, 1 H), 7.78 (s, 3 H), 7.77 (br s, 1 H), |
| | yl)pyridin-2- | | | 7.56 (d, 1 H), 7.18 (d, 2 H), 5.31 (s, 2 H), |
| | yl]methoxy}benzamide | | | 4.0 (d, 2 H), 3.50 (d, 2 H), 3.30 (dd, 2 H), 3.17 (d |
| | | | | 2 H), 2.79 (s, 3 H), 2.34 (s, 3 H) |

The following Example 156 was prepared in a similar fashion to Example 1 utilizing commercially available starting materials:

| | | | MS | |
|---|---|---|---|---|
| Ex. | Name | MW | (M + H⁺) | ¹H NMR ( [symbol] ppm) |
| 156 | N-[5-(1,2-dimethyl-1H- | 412.49 | 413 | 2.28 (s, 3 H), 2.61 (s, 3 H), 3.76 (s, 3 H), |
| | imidazol-4-yl)-2- | | | 5.3 (d, 2 H), 7.19 (d, 2 H), 7.43 (m, 2 H), 7.57 (m, |
| | methylphenyl]-4-(pyridin- | | | 2 H), 7.77 (s, 1 H), 7.90 (m, 1 H), 7.99 (d, 2 H |
| | 2-ylmethoxy)benzamide | | | 8.05 (s, 1 H), 8.63 (m, 1 H), 9.88 (br s, 1 H), |
| | | | | 14.57 (br s, 1 H) |

### Example 157

### Hedgehog Pathway Cellular Differentiation Assay

The ability of compounds of the invention to inhibit the Hedgehog pathway can be determined by the following cell differentiation assay.

C3H10T1/2 cells were plated into 384 well plates at a concentration of 5000 cells/well in DMEM/10% FBS. The following day the media was changed to 20% conditioned media (low serum media DMEM/2% FBS+Shh ligand). Compounds were solubilized in 100% DMSO to a concentration of 10 mM and then serially diluted three fold in 100% DMSO. The highest concentration in the cell plate was 30 μM and the lowest was 3 nM. The compounds were then added to the cells. Cell plates were incubated with the compound for 72 h and then assayed for alkaline phosphatase production using pNp as a substrate. Briefly, after 72 h of incubation, the media was aspirated from the cells and washed with 30 μl of PBS. PBS was aspirated off the cells and 15 μl of 1×RIPA cell lysis buffer is added on to the cells. The cell plates are then incubated at −80° C. for 30 minutes to insure proper cell lysis. The plates were then thawed back to RT. The substrate solution containing pNp at 1 mg/mL in diethanolamine buffer pH 9.8 was then added onto the lysed cells. The plates were incubated at 30° C. for color development and read at an absorbance of 405 nm. The percent inhibition and IC₅₀ value was then calculated from the absorbance data using standard procedures.

When tested in the above assay, exemplary compounds showed an IC₅₀ of less than about 30 μM. For example, the following results were obtained as shown in Table 2.

TABLE 2

| Example | IC₅₀ (μM) |
|---|---|
| 9 | 0.12 |
| 18 | 0.03 |
| 20 | <0.003 |

Percent inhibition at 3 μM for all examples disclosed herein according to the assay describe above are shown in Table 3.

TABLE 3

| | % Inhibition at |
|---|---|
| Example | 3 μM |
| 1 | 64.9 |
| 2 | 72.3 |
| 3 | 67.3 |
| 4 | 65.4 |
| 5 | 68.8 |
| 6 | 65.7 |
| 7 | 70.8 |
| 8 | 66.5 |
| 9 | 60.5 |
| 10 | 63.5 |
| 11 | 75.0 |
| 12 | 74.2 |
| 13 | 64.9 |
| 14 | 79.2 |
| 15 | 80.7 |
| 16 | 68.6 |
| 17 | 53.5 |
| 18 | 48.4 |
| 19 | 52.0 |
| 20 | 58.1 |
| 21 | 73.4 |
| 22 | 60.7 |
| 23 | 73.6 |
| 24 | 81.1 |
| 25 | 79.5 |
| 26 | 75.7 |
| 27 | 76.6 |
| 28 | 91.9 |
| 29 | 75.3 |
| 30 | 80.1 |
| 31 | 27.9 |
| 32 | 54.1 |
| 33 | 54.7 |
| 34 | 69.4 |
| 35 | 76.8 |
| 36 | 26.9 |
| 37 | 38.2 |
| 38 | 24.2 |
| 39 | 7.8 |
| 40 | 6.5 |
| 41 | 51.9 |
| 42 | 79.4 |
| 43 | 81.4 |
| 44 | 80.7 |
| 45 | 80.3 |
| 46 | 81.9 |
| 47 | 80.9 |
| 48 | 81.5 |
| 49 | 82.0 |
| 50 | 80.5 |
| 51 | 82.1 |
| 52 | 79.8 |
| 53 | 80.4 |
| 54 | 77.5 |
| 55 | 84.3 |
| 56 | 67.4 |
| 57 | 73.6 |
| 58 | 86.2 |
| 59 | 82.5 |
| 60 | 79.7 |
| 61 | 82.1 |
| 62 | 76.9 |
| 63 | 79.8 |
| 64 | 79.6 |
| 65 | 50.0 |
| 66 | 69.6 |
| 67 | 77.1 |
| 68 | 83.3 |
| 69 | 83.9 |
| 70 | 78.1 |
| 71 | 84.9 |
| 72 | — |
| 73 | 85.8 |
| 74 | 76.2 |
| 75 | 77.8 |
| 76 | 77.6 |
| 77 | 86.1 |
| 78 | 75.1 |
| 79 | 85.9 |
| 80 | 57.1 |
| 81 | 78.8 |
| 82 | 68.6 |
| 83 | 15.1 |
| 84 | 79.6 |
| 85 | 78.8 |
| 86 | 85.7 |
| 87 | 74.8 |
| 88 | 84.7 |
| 89 | 72.7 |
| 90 | 61.5 |
| 91 | 80.4 |
| 92 | 76.0 |
| 93 | 79.9 |
| 94 | 56.7 |
| 95 | 47.0 |
| 96 | 79.8 |
| 97 | 81.9 |
| 98 | 78.4 |
| 99 | 29.9 |
| 100 | 82.7 |
| 101 | 83.5 |
| 102 | 87.1 |
| 103 | 85.6 |
| 104 | — |
| 105 | 82.1 |
| 106 | 81.6 |
| 107 | 92.3 |
| 108 | 92.4 |
| 109 | 91.3 |
| 110 | 92.3 |
| 111 | −0.1 |
| 112 | 54.3 |
| 113 | 84.7 |
| 114 | 73.2 |
| 115 | 82.8 |
| 116 | 83.4 |
| 117 | 68.8 |
| 118 | 75.4 |
| 119 | 92.4 |
| 120 | 78.3 |
| 121 | 77.8 |
| 122 | 84.6 |
| 123 | 84.7 |
| 124 | 46.8 |
| 125 | 76.7 |
| 126 | 47.2 |
| 127 | 76.3 |
| 128 | 81.5 |
| 129 | 82.8 |
| 130 | 91.9 |
| 131 | 72.7 |
| 132 | 77.1 |
| 133 | 77.0 |
| 134 | 77.3 |
| 135 | 76.9 |
| 136 | 77.0 |
| 137 | 76.5 |
| 138 | 42.2 |
| 139 | 84.1 |
| 140 | 85.5 |
| 141 | 85.3 |
| 142 | 31.3 |
| 143 | 62.3 |
| 144 | 8.8 |
| 145 | 67.1 |
| 146 | 17.1 |
| 147 | 19.3 |
| 148 | 9.1 |
| 149 | 63.4 |
| 150 | 19.4 |
| 151 | 21.9 |
| 152 | 45.3 |
| 153 | 53.7 |
| 154 | 17.6 |
| 155 | 83.3 |
| 156 | 86.2 |

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents were considered to be within the scope of this invention and are covered by the following claims. The contents of all references, issued patents, and published patent applications cited throughout this application are hereby incorporated by reference.

1. A compound of formula IA 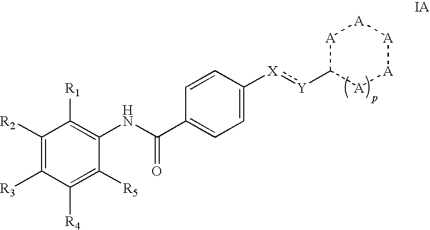
wherein [symbol]
represents a single bond or a double bond; [symbol]
represents a single bond, a double bond, a triple bond, or when X or Y is a direct bond [symbol] represents the absence of a bond;
R₁, R₂, R₃, and R₄ are each independently selected from the group consisting of hydrogen, C₁₋₆alkoxy, C₁₋₆alkoxyC₁₋₆alkyl, C₁₋₆alkyl, aminoC₁₋₆alkyl, C₃₋₈cycloalkyl, cyano, haloC₁₋₆alkyl, halogen, hydroxy, sulfonyl, sulfide, and thio,
with the proviso that either R₂ or R₃ is Z; 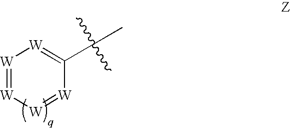
each W is independently selected from the group consisting of CR₁₀, NR₁₀, N, O, and S, where R₁₀ is selected from the group consisting of hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, C₃₋₆cycloalkyl, hydroxy, hydroxyC₁₋₆alkyl, nitro, sulfide, sulfonamido, and sulfonyl, or
two adjacent W atoms can be taken together with their R₁₀ substituents to form a fused second ring, wherein the second ring is selected from the group consisting of aryl, C₃₋₈cycloalkyl, a 5- or 6-membered heteroaryl, and a 5- or 6-membered heterocyclyl;
q is 0 or 1, where
if q is 0 and two adjacent W atoms taken together with their R₁₀ substituents form a bicycle selected from the group consisting of benzimidazolyl, benzoxazolyl, benzothiazolyl, and oxazolopyridyl, then at least one A is N,
if q is 1, two W are N, and two adjacent W atoms taken together with their R₁₀ substituents form a quinoxalinyl, then at least one A is N, and
if q is 1 and each W is CR₁₀, then two adjacent W atoms are taken together with their R₁₀ substituents to form a second ring selected from the group consisting of a 5- or 6-membered heteroaryl and a 5- or 6-membered heterocyclyl;
R₅ is selected from the group consisting of alkyl, haloC₁₋₆alkyl, and halogen;
R₆, R₇, R₈ and R₉ are each independently selected from the group consisting of hydrogen, C₁₋₆alkyl, amino, C₃₋₈cycloalkyl, C₁₋₆alkoxy, cyano, haloC₁₋₆alkyl, halogen, sulfide, sulfonyl, and sulfonamido;
when joined by a single bond, X and Y are each independently selected from the group consisting of O, S, SO₂, NR₁₁, and CR₁₁R₁₂, or one of X and Y can be a direct bond,
when joined by a double bond, X and Y are each independently CR₁₁, and
when joined by a triple bond, X and Y are each C;
each R₁₁ and R₁₂ are each independently selected from the group consisting of hydrogen, C₁₋₆alkoxy, C₁₋₆alkyl, amino, cyano, haloC₁₋₆alkyl, halogen, and sulfide;
each A is selected from the group consisting of CR₁₃, CR₁₃R₁₃, NR₁₃, N, O, and S;
each R₁₃ is selected from the group consisting of hydrogen, C₁₋₆alkoxy, C₁₋₆alkoxyamino, C₁₋₆alkoxyC₁₋₆alkyl, C₁₋₆alkoxycarbonyl, C₁₋₆alkyl, C₁₋₆alkylamino, amidino, amido, amino, aminoC₁₋₆alkylamino, aryl, aryloxy, carboxamido, C₃₋₈cycloalkyl, C₃₋₈cycloalkylC₁₋₆alkoxy, cyano, haloC₁₋₆alkyl, halogen, heterocyclyl, heterocyclylC₁₋₆alkyl, heterocyclylC₁₋₆alkoxy, hydroxy, hydroxyC₁₋₆alkyl, hydroxyC₁₋₆alkoxy, nitro, sulfide, sulfonamido, and sulfonyl;
p is 0 or 1, where
if p is 0, then two adjacent A atoms can be taken together with their R₁₃ substituents to form a fused second ring, wherein the second ring is selected from the group consisting of aryl, 6-membered heteroaryl and 6-membered heterocyclyl, and
if p is 1, then two adjacent A atoms can be taken together with their R₁₃ substituents to form a fused second ring, wherein the second ring is selected from the group consisting of aryl, 5- or 6-membered heteroaryl and 5- or 6-membered heterocyclyl;
or a pharmaceutically acceptable salt thereof.

2. A compound of formula II 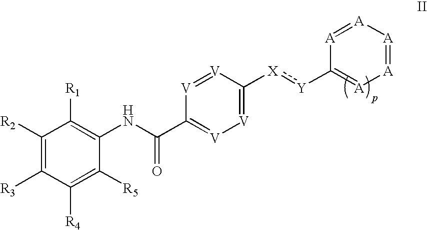
wherein
R₁, R₂, R₃, and R₄ are each independently selected from the group consisting of hydrogen, C₁₋₆alkoxy, C₁₋₆alkoxyC₁₋₆alkyl, C₁₋₆alkyl, aminoC₁₋₆alkyl, C₃₋₈cycloalkyl, cyano, haloC₁₋₆alkyl, halogen, hydroxy, sulfonyl, sulfide, and thio,
with the proviso that either R₂ or R₃ is Z; 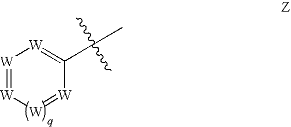
each W is independently selected from the group consisting of CR₁₀, NR₁₀, N, O, and S, where R₁₀ is selected from the group consisting of hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, C₃₋₆cycloalkyl, hydroxy, hydroxyC₁₋₆alkyl, nitro, sulfide, sulfonamido, and sulfonyl, or
two adjacent W atoms can be taken together with their R₁₀ substituents to form a fused second ring, wherein the second ring is selected from the group consisting of aryl, C₃₋₈cycloalkyl, a 5- or 6-membered heteroaryl, and a 5- or 6-membered heterocyclyl;
q is 0 or 1, where
if q is 0 and two adjacent W atoms taken together with their R₁₀ substituents form a bicycle selected from the group consisting of benzimidazolyl, benzoxazolyl, benzothiazolyl, and oxazolopyridyl, then at least one A is N,
if q is 1, two W are N, and two adjacent W atoms taken together with their R₁₀ substituents form a quinoxalinyl, then at least one A is N, and
if q is 1 and each W is CR₁₀, then two adjacent W atoms are taken together with their R₁₀ substituents to form a second ring selected from the group consisting of a 5- or 6-membered heteroaryl and a 5- or 6-membered heterocyclyl;
R₅ is selected from the group consisting of alkyl, haloC₁₋₆alkyl, and halogen;
when joined by a single bond, X and Y are each independently selected from the group consisting of O, S, SO₂, NR₁₁, and CR₁₁R₁₂, or one of X and Y can be a direct bond,
when joined by a double bond, X and Y are each independently CR₁₁, and
when joined by a triple bond, X and Y are each C;
each R₁₁ and R₁₂ are each independently selected from the group consisting of hydrogen, C₁₋₆alkoxy, C₁₋₆alkyl, amino, cyano, haloC₁₋₆alkyl, halogen, and sulfide;
each A is selected from the group consisting of CR₁₃, NR₁₃, N, O, and S;
each R₁₃ is selected from the group consisting of hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, C₃₋₈cycloalkyl, cyano, haloC₁₋₆alkyl, halogen, heterocyclylC₁₋₆alkyl, hydroxy, hydroxyC₁₋₆alkyl, nitro, sulfide, sulfonamido, and sulfonyl;
each V is independently selected from the group consisting of CR₁₄ and N;
each R₁₄ is selected from the group consisting of hydrogen, C₁₋₆alkoxy, C₁₋₆alkoxyC₁₋₆alkyl, C₁₋₆alkoxycarbonyl, C₁₋₆alkyl, amidino, amido, amino, aryl, carboxamido, cyano, haloC₁₋₆alkyl, halogen, heterocyclylC₁₋₆alkyl, hydroxy, hydroxyC₁₋₆alkyl, nitro, sulfide, sulfonamido, and sulfonyl;
p is 0 or 1, where
if p is 0, then two adjacent A atoms can be taken together with their R₁₃ substituents to form a fused second ring, wherein the second ring is selected from the group consisting of aryl, 6-membered heteroaryl and 6-membered heterocyclyl; and
if p is 1, then two adjacent A atoms can be taken together with their R₁₃ substituents to form a fused second ring, wherein the second ring is selected from the group consisting of aryl, 5- or 6-membered heteroaryl and 5- or 6-membered heterocyclyl,
or a pharmaceutically acceptable salt thereof.

3. A compound of formula III 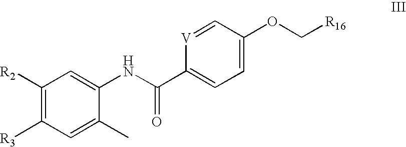
wherein
V is N or CH;
R₂ is selected from the group consisting of pyrazolyl, imidazolyl, benzoimidazol, thiazolyl, pyridyl, triazolyl, purinyl, and quinoxalinyl, wherein R₂ is optionally substituted with one or more R₁₅;
R₁₅ may be selected from the group consisting of alkyl, nitro, aryl, heteroaryl wherein R₁₅ may be optionally substituted with halo, alkyl, alkoxy, alkylthio, aryl, and heteroaryl;
R₃ is selected from the group consisting of hydrogen and alkyl;
R₁₆ is selected from the group consisting of aryl and heterocyclyl wherein R₁₆ is optionally substituted with R₁₇; and
R₁₇ is selected from the group consisting of halo, alkyl, alkoxy, alkylthio, wherein R₁₇ is optionally substituted with aryl or heteroaryl,
or a pharmaceutically acceptable salt thereof.

4. The compound of claim 3, wherein one of R₂ or R₃ is imidazolyl.

5. The compound of claim 3, wherein R₁₆ is pyridyl or phenyl.

6. A compound of formula IV 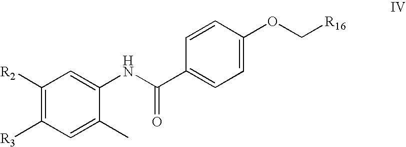
wherein
R₂ is selected from the group consisting of thiazol-2-yl, quinoxalin-2-yl, phenyl, benzothiazol-2-yl, 7H-purin-6-yl, 6-aminopyridazin-3-yl, 6-amino-2-pyridyl, 5-nitro-1H-benzoimidazol-2-yl, 5-methyl-3H-imidazol-4-yl, 5-methyl-1H-imidazol-4-yl, 5-methyl-1,3,4-oxadiazol-2-yl, 5-methyl-1,2,4-oxadiazol-3-yl, 5-ethoxycarbonyl-4-methyl-thiazol-2-yl, 5-aminopyrazin-2-yl, 5-amino-2-pyridyl, 5-[(4-methylpiperazin-1-yl)methyl]thiazol-2-yl, 5,7-diazabicyclo[4.3.0]nona-2,4,8,10-tetraen-4-yl, 5-(trifluoromethyl)-2H-pyrazol-3-yl, 5-(pyrazol-1-ylmethyl)thiazol-2-yl, 5-(morpholinomethyl)thiazol-2-yl, 5-(hydroxymethyl)-1-methyl-imidazol-4-yl, 4-thiazol-2-yl-1H-imidazol-2-yl, 4-thia-1,6-diazabicyclo[3.3.0]octa-2,5,7-trien-7-yl, 4-tert-butyl-1H-imidazol-2-yl, 4-pyridyl, 4-phenyl-1H-imidazol-2-yl, 4-methyl-3H-imidazol-2-yl, 4-methyl-1H-imidazol-2-yl, 4-ethyl-1H-imidazol-2-yl, 4-cyclopropyl-1H-imidazol-2-yl, 4,5-dimethyl-1,2,4-triazol-3-yl, 4-(trifluoromethyl)-3H-imidazol-2-yl, 4-(hydroxymethyl)-1H-imidazol-2-yl, 4-(4-pyrrolidin-1-ylphenyl)-1H-imidazol-2-yl, 4-(3-pyridyl)-1H-imidazol-2-yl, 3-pyridyl, 3-methylimidazol-4-yl, 2-pyridyl, 2-methylpyrazol-3-yl, 2-methyl-1H-imidazol-4-yl, 2,4-dimethylthiazol-5-yl, 2,3-dimethylimidazol-4-yl, 1-methylpyrazol-4-yl, 1-methylimidazol-4-yl, 1-methylimidazol-2-yl, 1-methyl-5-(methylaminomethyl)imidazol-4-yl, 1-isobutylpyrazol-4-yl, 1H-triazol-4-yl, 1H-imidazol-4-yl, 1H-imidazol-2-yl, 1H-benzoimidazol-2-yl, 1-[(3-bromo-2-pyridyl)methyl]imidazol-2-yl, 1,5-dimethylimidazol-2-yl, 1,4-dimethylimidazol-2-yl, 1,3,5-trimethylpyrazol-4-yl, 1,2-dimethylimidazol-4-yl;
R₃ is selected from the group consisting of hydrogen, methyl, and 1H-benzoimidazol-2-yl; and
R₁₆ is selected from the group consisting of 2-cyanophenyl, 2-methoxyphenyl, 3,4-dimethoxy-2-pyridyl, 3,5-dimethoxyphenyl, 3-cyanophenyl, 3-methoxyphenyl, 4-fluorophenyl, 4-methylsulfonylphenyl, 6-chlorobenzo[1,3]dioxol-5-yl, 2-(trifluoromethyl)phenyl, 3-(2-morpholinoethoxy)phenyl, 4-(hydroxymethyl)phenyl, and 2-pyridyl,
or a pharmaceutically acceptable salt thereof.

7. A compound of formula V 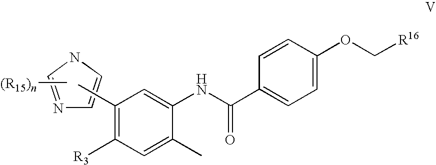
wherein
n is 0, 1, 2, or 3;
R₃ is selected from the group consisting of hydrogen, halogen, and alkyl;
R₁₅ is selected from the group consisting of halogen, hydroxyl, alkyl, alkoxyl, alkoxycarbonyl, sulfinyl, sulfonyl, cyano, cycloalkyl, aryl or a heterocyclyl wherein each R₁₅ is optionally substituted with hydroxyl, halogen, amino, nitro, alkyl, sulfonyl, cyano, alkoxyl or heterocyclyl;
R₁₆ is selected from the group consisting of aryl and heterocyclyl wherein R₁₆ is optionally substituted with R₁₇; and
R₁₇ is selected from the group consisting of halo, alkyl, alkoxy, alkylthio, wherein R₁₇ is optionally substituted with aryl or heteroaryl,
or a pharmaceutically acceptable salt thereof.

8. The compound of claim 7, wherein R₁₅ is halogen, optionally substituted alkyl, aryl, heterocyclyl, or cycloalkyl.

9. A pharmaceutical composition comprising one or more of the compounds according to claim 1, formulated together with one or more pharmaceutically acceptable carriers.

10. A method for inhibiting the Hedgehog pathway comprising administering to a subject a therapeutically effective amount of one or more of the compounds according to claim 1, such that the Hedgehog pathway is inhibited.

11. A method of reducing cell proliferation, differentiation and/or affecting stromal microenvironment modulation comprising administering to a subject a therapeutically effective amount of one or more of the compounds according to claim 1, thereby reducing cell proliferation, differentiation and/or affecting stromal microenvironment modulation in the subject.

12. The method of claim 11, wherein the cell is a stromal cell.

13. The method of claim 11, wherein the cell is a cancer cell.

14. The method of claim 11, wherein the cell is a stem cell.

15. The method of claim 14, where the stem cell is a cancer stem cell.
